(19) United States
(12) Patent Application Publication
(10) Pub. No.: US 2020/0206212 A1
(43) Pub. Date: Jul. 2, 2020

(54) COMPOSITIONS AND METHODS OF USE OF 2-(4-CHLOROPHENYL)-N-((2-(2,6-DIOXOPIPERIDIN-3-YL)-1-OXOISOINDOLIN-5-YL)METHYL)-2,2-DIFLUOROACETAMIDE

(71) Applicant: Celgene Corporation, Summit, NJ (US)

(72) Inventors: Rowena Fernandez Choudrie, Summit, NJ (US); Willard Foss, San Diego, CA (US); Che-Hsiung Hsu, San Diego, CA (US); Amol Mungikar, Summit, NJ (US); Yu Pu, Summit, NJ (US)

(21) Appl. No.: 16/730,591

(22) Filed: Dec. 30, 2019

Related U.S. Application Data
(60) Provisional application No. 62/787,034, filed on Dec. 31, 2018.

(51) Int. Cl. A61K 31/454; A61K 45/06; A61K 31/53; A61K 47/42; A61K 47/26; A61K 47/12; A61K 9/19

(52) U.S. Cl. CPC A61K 31/454; A61K 45/06; A61K 31/53; A61K 9/19; A61K 47/26; A61K 47/12; A61K 47/42

(57) ABSTRACT

Provided herein are formulations and methods of use of 2-(4-chlorophenyl)-N-((2-(2,6-dioxopiperidin-3-yl)-1-oxoisoindolin-5-yl)methyl)-2,2-difluoroacetamide or a stereoisomer or mixture of stereoisomers, pharmaceutically acceptable salt, tautomer, prodrug, isotopologue, solvate, hydrate, co-crystal, clathrate, or polymorph thereof.

### RELATED APPLICATIONS

This application claims the benefit of U.S. Provisional Application No. 62/787,034, filed Dec. 31, 2018, the disclosure of which is incorporated herein by reference in its entirety.

### FIELD

Provided are formulations and dosage forms of 2-(4-chlorophenyl)-N-((2-(2,6-dioxopiperidin-3-yl)-1-oxoisoindolin-5-yl)methyl)-2,2-difluoroacetamide or a stereoisomer or a mixture of stereoisomers, pharmaceutically acceptable salt, tautomer, prodrug, isotopologue, solvate, hydrate, co-crystal, clathrate, or polymorph thereof. Methods of using the formulations and dosage forms for treating, managing, and/or preventing cancer are also provided herein. Thus, provided herein are said formulations and dosage forms for use in methods of treating, managing, and/or preventing cancer.

### BACKGROUND

2-(4-Chlorophenyl)-N-((2-(2,6-dioxopiperidin-3-yl)-1-oxoisoindolin-5-yl)methyl)-2,2-difluoroacetamide or a stereoisomer or mixture of stereoisomers, pharmaceutically acceptable salt, tautomer, prodrug, solvate, hydrate, co-crystal, clathrate, or polymorph thereof has been shown to have anti-cancer activities. Exemplary formulations of the compound are disclosed in U.S. Pat. No. 10,052,315 B2 and U.S. application Ser. No. 16/024,581, filed on Jun. 29, 2018.

There is a need for further formulations of 2-(4-chlorophenyl)-N-((2-(2,6-dioxopiperidin-3-yl)-1-oxoisoindolin-5-yl)methyl)-2,2-difluoroacetamide or a stereoisomer or mixture of stereoisomers, pharmaceutically acceptable salt, tautomer, prodrug, solvate, hydrate, co-crystal, clathrate, or polymorph thereof for use in methods of treatment of cancer.

### BRIEF SUMMARY

Compound 1 used in the formulations and methods herein is described in U.S. Pat. No. 9,499,514 and International Publication No. WO 2016/007848, the disclosures of each which are incorporated herein by reference in their entireties. In one embodiment, Compound 1 is polymorph Form A, Form B, Form C, Form D, Form E or an amorphous form of 2-(4-chlorophenyl)-N-((2-(2,6-dioxopiperidin-3-yl)-1-oxoisoindolin-5-yl)methyl)-2,2-difluoroacetamide. In one embodiment, Compound 1 is polymorph Form C of 2-(4-chlorophenyl)-N-((2-(2,6-dioxopiperidin-3-yl)-1-oxoisoindolin-5-yl)methyl)-2,2-difluoroacetamide. The polymorphs of 2-(4-chlorophenyl)-N-((2-(2,6-dioxopiperidin-3-yl)-1-oxoisoindolin-5-yl)methyl)-2,2-difluoroacetamide are described herein and in U.S. Pat. No. 10,189,808, the disclosure of which is incorporated herein by reference in its entirety.

In one embodiment, provided herein are formulations comprising Compound 1 and mannitol. In one embodiment, provided herein are formulations comprising Compound 1, mannitol and a citrate buffer. In one embodiment, provided herein are formulations comprising Compound 1 in an amount of about 1% to 1.3%, a citrate buffer in an amount of about 9% to 12%, and mannitol in an amount of about 85% to 90% based on total weight of the formulation. In one embodiment, the citrate buffer comprises citric acid monohydrate and sodium citrate dihydrate.

In one embodiment, provided herein are formulations comprising Compound 1 in an amount of about 1% to 1.3%, citric acid monohydrate in an amount of about 4% to 7.5%, sodium citrate dihydrate in an amount of about 3% to 5.5%, and mannitol in an amount of about 85% to 90% based on total weight of the formulation.

In certain embodiments, provided herein are formulations comprising Compound 1 and human albumin. In certain embodiments, provided herein are formulations comprising Compound 1, human albumin and sucrose. In certain embodiments, provided herein are formulations comprising Compound 1, human albumin, sucrose and mannitol. In certain embodiments, provided herein are formulations comprising Compound 1, human albumin, trehalose and mannitol. In certain embodiment, provided herein are formulations comprising Compound 1, a citrate buffer, human albumin, and sucrose. In certain embodiment, provided herein are formulations comprising Compound 1, a citrate buffer, human albumin, mannitol and sucrose. In certain embodiment, provided herein are formulations comprising Compound 1, a citrate buffer, human albumin, and trehalose. In certain embodiment, provided herein are formulations comprising Compound 1, a citrate buffer, human albumin, mannitol and trehalose. In one embodiment, the citrate buffer comprises citric acid anhydrous and sodium citrate dihydrate.

In one embodiment, the methods provided herein comprise administering a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one embodiment, the methods provided herein comprise administering a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In certain embodiments, the formulations provided herein comprise a solid form of 2-(4-chlorophenyl)-N-((2-(2,6-dioxopiperidin-3-yl)-1-oxoisoindolin-5-yl)methyl)-2,2-difluoroacetamide. In certain embodiments, the formulations provided herein comprise an amorphous form of 2-(4-chlorophenyl)-N-((2-(2,6-dioxopiperidin-3-yl)-1-oxoisoindolin-5-yl)methyl)-2,2-difluoroacetamide.

In certain embodiments, provided herein is a unit dosage form comprising a formulation provided herein.

In one aspect, the formulations containing therapeutically effective concentrations of Compound 1 are administered to an individual exhibiting the symptoms of the disease or disorder to be treated. The amounts are effective to ameliorate or eliminate one or more symptoms of the disease or disorder.

Further provided is a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like.

These and other aspects of the subject matter described herein will become evident upon reference to the following detailed description.

### BRIEF DESCRIPTION OF THE DRAWINGS

FIG. 1 provides a flow diagram for preparation of an exemplary formulation.

FIG. 2 provides a typical chromatogram of Compound 1 (labeled API) in human albumin formulations.

FIG. 3 provides a typical chromatogram of related impurities in Compound 1 in human albumin formulations.

FIG. 4 provides a differential scanning calorimetry plot obtained with a standard heat flow (10° C./min) showing the nucleation onset temperature for the human albumin formulation of Example 4.

FIG. 5 provides a differential scanning calorimetry plot obtained with a standard heat flow (10° C./min) showing the glass transition temperature for the formulation of Example 4.

FIG. 6 provides a differential scanning calorimetry plot obtained with a standard heat flow (10° C./min) showing the ice melt temperature for the formulation of Example 4.

FIG. 7 provides a differential scanning calorimetry plot obtained with a modulated heat flow showing the nucleation onset temperature for the human albumin formulation of Example 4.

FIG. 8 provides a differential scanning calorimetry plot obtained with a modulated heat flow showing the glass transition temperature for the human albumin formulation of Example 4.

FIG. 9 provides a differential scanning calorimetry plot obtained with a modulated heat flow showing the ice melt temperature for the human albumin formulation of Example 4.

FIG. 10 provides a differential scanning calorimetry plot obtained with a modulated heat flow showing the nucleation onset temperature for 5% human albumin.

FIG. 11 provides a differential scanning calorimetry plot obtained with a modulated heat flow showing the melt curve for 5% human albumin.

FIG. 12 provides a differential scanning calorimetry plot obtained with a modulated heat flow showing the ice melt temperature for 5% human albumin.

FIG. 13 demonstrates the increase in related impurities with time in solutions of Formulation 16 stored at different temperatures and relative humidities.

FIG. 14 demonstrates the drop in Compound 1 concentration with time in solutions of Formulation 16 stored at different temperatures and relative humidities.

FIGS. 15A-15F show the effect of 8 months storage at 40° C./75% relative humidity on Compound 1 concentration in Formulations 7-12, respectively.

FIGS. 16A, 16B and 16C show the effect of 1 year storage at 40° C./75% relative humidity on Compound 1 concentration in Formulations 8, 11 and 12, respectively.

FIG. 17 provides an HPLC chromatogram showing monomer, dimer, oligomer, and polymer fractions of human album.

FIGS. 18A-18F show the effect of 8 months storage at 40° C./75% relative humidity on total human albumin concentration in terms of monomer, dimer, oligomer, and polymer fractions in Formulations 7-12, respectively.

FIGS. 19A, 19B and 19C show the effect of 8 months storage at 40° C./75% relative humidity on total human albumin concentration in terms of monomer, dimer, oligomer, and polymer fractions in Formulations 8, 11 and 12, respectively.

FIGS. 20A, 20B and 20C provide plots for solubility of Compound 1 in formic acid (FA) and acetic acid (AcOH) mixtures.

FIG. 21 provides a flow diagram for the preparation of formulations A, B, C and D.

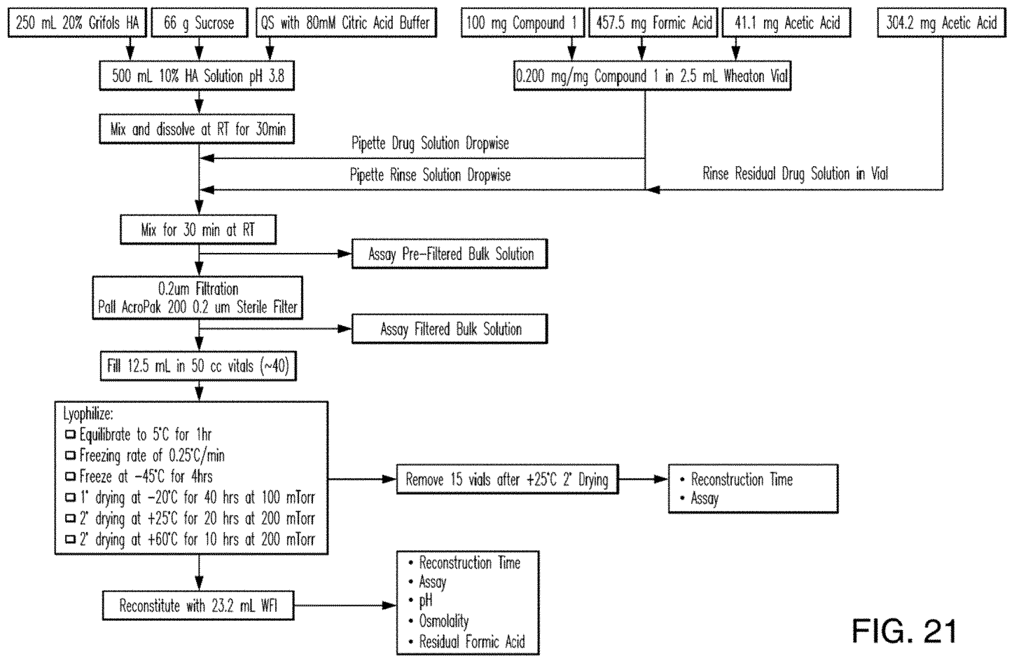

FIG. 22 provides a schematic for the preparation of samples to study the effect of pH, fill volume and drug content on reconstitution time for formulations A, B, C and D.

FIG. 23 provides a flow diagram for the preparation of Formulation 19 for the monkey pharmacokinetic study.

FIG. 24 provides pharmacokinetic data for Formulation lb and Formulation 19 in monkeys.

FIG. 25 provides a flow diagram for the preparation of a large scale batch of Formulation 24.

### DETAILED DESCRIPTION

### Definitions

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In general, the technical teaching of one embodiment can be combined with that disclosed in other embodiments provided herein.

The use of the word “a” or “an” when used in conjunction with the term “comprising” in the claims and/or the specification can mean “one”, but it is also consistent with the meaning of “one or more”, “at least one” and “one or more than one.”

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the terms “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive. E.g., “treating, preventing or managing” or similar listings means: “treating; preventing; managing; treating and preventing; treating and managing; preventing and managing; treating, preventing and managing”.

The term “Compound 1” refers to “2-(4-chlorophenyl)-N-((2-(2,6-dioxopiperidin-3-yl)-1-oxoisoindolin-5-yl)methyl)-2,2-difluoroacetamide” having the structure:

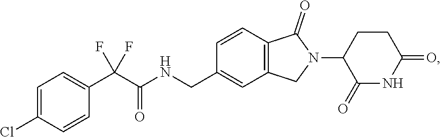 [SMILES: O=C1CCC(N2Cc3cc(CNC(=O)C(F)(F)c4ccc(Cl)cc4)ccc3C2=O)C(=O)N1]

and its stereoisomers or mixture of stereoisomers, pharmaceutically acceptable salts, tautomers, prodrug, isotopologue, solvates, hydrates, co-crystals, clathrates, or polymorphs thereof. In certain embodiments, Compound 1 refers to 2-(4-chlorophenyl)-N-((2-(2,6-dioxopiperidin-3-yl)-1-oxoisoindolin-5-yl)methyl)-2,2-difluoroacetamide and its tautomers. In certain embodiments, Compound 1 refers to a polymorph of 2-(4-chlorophenyl)-N-((2-(2,6-dioxopiperidin-3-yl)-1-oxoisoindolin-5-yl)methyl)-2,2-difluoroacetamide, such as Form A, B, C, D, or E, or a mixture thereof. In certain embodiments, Compound 1 refers to polymorph Form C of 2-(4-chlorophenyl)-N-((2-(2,6-dioxopiperidin-3-yl)-1-oxoisoindolin-5-yl)methyl)-2,2-difluoroacetamide. In certain embodiments, Compound 1 refers to an amorphous form of 2-(4-chlorophenyl)-N-((2-(2,6-dioxopiperidin-3-yl)-1-oxoisoindolin-5-yl)methyl)-2,2-difluoroacetamide. In one embodiment, the stereoisomer is an enantiomer.

Unless specifically stated otherwise, where a compound may assume alternative tautomeric, regioisomeric and/or stereoisomeric forms, all alternative isomers are intended to be encompassed within the scope of the claimed subject matter. For example, where a compound can have one of two tautomeric forms, it is intended that both tautomers be encompassed herein.

Thus, the compounds herein may be enantiomerically pure, or be stereoisomeric or diastereomeric mixtures. As used herein and unless otherwise indicated, the term “stereoisomerically pure” means a composition that comprises one stereoisomer of a compound and is substantially free of other stereoisomers of that compound. For example, a stereoisomerically pure composition of a compound having one chiral center will be substantially free of the opposite enantiomer of the compound. A stereoisomerically pure composition of a compound having two chiral centers will be substantially free of other diastereomers of the compound. A typical stereoisomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. A stereoisomerically pure compound as used herein comprises greater than about 80% by weight of one stereoisomer of the compound, more preferably greater than about 90% by weight of one stereoisomer of the compound, even more preferably greater than about 95% by weight of one stereoisomer of the compound, and most preferably greater than about 97% by weight of one stereoisomer of the compound. As used herein and unless otherwise indicated, the term “stereoisomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereoisomerically pure composition of a compound having one chiral center. Similarly, the term “stereoisomerically enriched” means a stereoisomerically enriched composition of a compound having one chiral center. As used herein, stereoisomeric or diastereomeric mixtures means a composition that comprises more than one stereoisomer of a compound. A typical stereoisomeric mixture of a compound comprises about 50% by weight of one stereoisomer of the compound and about 50% by weight of other stereoisomers of the compound, or comprises greater than about 50% by weight of one stereoisomer of the compound and less than about 50% by weight of other stereoisomers of the compound, or comprises greater than about 45% by weight of one stereoisomer of the compound and less than about 55% by weight of the other stereoisomers of the compound, or comprises greater than about 40% by weight of one stereoisomer of the compound and less than about 60% by weight of the other stereoisomers of the compound, or comprises greater than about 35% by weight of one stereoisomer of the compound and less than about 65% by weight of the other stereoisomers of the compound.

It should also be noted the compounds herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer therapeutic agents, research reagents, e.g., binding assay reagents, and diagnostic agents, e.g., in vivo imaging agents. All isotopic variations of the Compound 1 as described herein, whether radioactive or not, are intended to be encompassed within the scope of the embodiments provided herein. In some embodiments, there are provided isotopologues of Compound 1, for example, the isotopologues are deuterium, carbon-13, and/or nitrogen-15 enriched Compound 1. As used herein, “deuterated”, means a compound wherein at least one hydrogen (H) has been replaced by deuterium (indicated by D or ²H), that is, the compound is enriched in deuterium in at least one position.

It is understood that, independently of stereomerical or isotopic composition, each compound referred to herein can be provided in the form of any of the pharmaceutically acceptable salts discussed herein. Equally, it is understood that the isotopic composition may vary independently from the stereomerical composition of each compound referred to herein. Further, the isotopic composition, while being restricted to those elements present in the respective compound or salt thereof, may otherwise vary independently from the selection of the pharmaceutically acceptable salt of the respective compound.

As used herein, API refers to Compound 1. In certain embodiments, API refers to 2-(4-chlorophenyl)-N-((2-(2,6-dioxopiperidin-3-yl)-1-oxoisoindolin-5-yl)methyl)-2,2-difluoroacetamide.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. 1972, 11:942-944).

As used herein, and unless otherwise specified, the term “lyophilize” refers to the process of isolating a solid substance from solution and/or removal of solvent. In some embodiments, this may be achieved by various techniques known to one of skill in the art, including, for example, evaporation (e.g., under vacuum, for example by freeze drying, and/or freezing the solution and vaporizing the frozen solvent under vacuum or reduced pressure conditions, etc.)

As used herein, “reconstituted aqueous solution” or “reconstituted aqueous composition” or “reconstituted aqueous formulation” refers to an aqueous solution obtained by dissolving a lyophilized formulation provided herein in an aqueous solvent.

The term “aqueous diluent” used herein refers to an aqueous liquid capable of being included in a parenteral formulation. Such aqueous diluents can include, for example, water, saline, ½ normal saline or dextrose if desired, as well as any of the known ancillary preservatives or excipients commonly found as part of parenteral formulations. Exemplary aqueous diluents include water, 5% dextrose solution, and the like.

As used herein, “collapse temperature” or “Tc” refers to the temperature at which material in an amorphous state weakens to the point of instability, which leads to incomplete drying, inadequate stability in reconstitution and poor product appearance.

As used herein, “glass transition” or “Tg′” refers to the temperature at which a rigid, amorphous glass changes viscosity to form a flowing mass. A Tg′ can be determined by differential scanning calorimetry.

As used herein, “nucleation temperature” or “Tnuc′” refers to the temperature at which freezing or ice crystal formation begins.

As used herein, “eutectic temperature” or “Teu′” refers to the maximum temperature that a crystalline material can withstand during primary drying without loss of structure.

As used herein, and unless otherwise specified, the term “parenteral” includes subcutaneous, intravenous, intramuscular, intra-artricular, intra-synovial, intrasternal, intrathecal, intrahepatic, intralesional and intracranial injection or infusion techniques.

As used herein, and unless otherwise specified, the expression “unit dose” refers to a physically discrete unit of a formulation appropriate for a subject to be treated (e.g., for a single dose); each unit containing a predetermined quantity of an active agent selected to produce a desired therapeutic effect (it being understood that multiple doses may be required to achieve a desired or optimum effect), optionally together with a pharmaceutically acceptable carrier, which may be provided in a predetermined amount. The unit dose may be, for example, a volume of liquid (e.g. an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present disclosure will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

As used herein, the term “solid form” refers a crystal form or an amorphous form or a mixture thereof of 2-(4-chlorophenyl)-N-((2-(2,6-dioxopiperidin-3-yl)-1-oxoisoindolin-5-yl)methyl)-2,2-difluoroacetamide or a stereoisomer or mixture of stereoisomers, pharmaceutically acceptable salt, tautomer, prodrug, isotopologue, solvate, hydrate, co-crystal, clathrate, or polymorph thereof.

As used herein, unless otherwise specified, the term “pharmaceutically acceptable salt(s),” as used herein includes, but is not limited to, salts of acidic or basic moieties of Compound 1. Basic moieties are capable of forming a wide variety of salts with various inorganic and organic acids. The acids that can be used to prepare pharmaceutically acceptable acid addition salts of such basic compounds are those that form non-toxic acid addition salts, e.g., salts containing pharmacologically acceptable anions. Suitable organic acids include, but are not limited to, maleic, fumaric, benzoic, ascorbic, succinic, acetic, formic, oxalic, propionic, tartaric, salicylic, citric, gluconic, lactic, mandelic, cinnamic, oleic, tannic, aspartic, stearic, palmitic, glycolic, glutamic, gluconic, glucaronic, saccharic, isonicotinic, methanesulfonic, ethanesulfonic, p-toluenesulfonic, benzenesulfonic acids, or pamoic (e.g., 1,1′-methylene-bis-(2-hydroxy-3-naphthoate) acids. Suitable inorganic acids include, but are not limited to, hydrochloric, hydrobromic, hydroiodic, sulfuric, phosphoric, or nitric acids. Compounds that include an amine moiety can form pharmaceutically acceptable salts with various amino acids, in addition to the acids mentioned above. Chemical moieties that are acidic in nature are capable of forming base salts with various pharmacologically acceptable cations. Examples of such salts are alkali metal or alkaline earth metal salts and, particularly, calcium, magnesium, sodium, lithium, zinc, potassium, or iron salts.

As used herein, and unless otherwise specified, the term “solvate” means a compound provided herein or a salt thereof that further includes a stoichiometric or non-stoichiometric amount of solvent bound by non-covalent intermolecular forces. Where the solvent is water, the solvate is a hydrate.

As used herein and unless otherwise indicated, the term “prodrug” means a derivative of a compound that can hydrolyze, oxidize, or otherwise react under biological conditions (in-vitro or in-vivo) to provide the compound. Examples of prodrugs include, but are not limited to, derivatives of compounds described herein (e.g., Compound 1) that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, ½ normal saline, sucrose, glucose, bulking agents, buffers, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R.C., Shesky P.J., and Quinn M. E., 6^{th} Ed., The Pharmaceutical Press, RPS Publishing (2009). The terms “bulking agent”, and “buffer” are used in accordance with the plain and ordinary meaning within the art.

As used herein, and unless otherwise specified, the term “about,” when used in connection with doses, amounts, or weight percent of ingredients of a composition or a dosage form, means dose, amount, or weight percent that is recognized by those of ordinary skill in the art to provide a pharmacological effect equivalent to that obtained from the specified dose, amount, or weight percent is encompassed. Specifically, the term “about” contemplates a dose, amount, or weight percent within 30%, 25%, 20%, 15%, 10%, or 5% of the specified dose, amount, or weight percent is encompassed.

As used herein, and unless otherwise specified, the term “stable,” when used in connection with a liquid formulation or a dosage form, means that the active ingredient of the formulation or dosage form remains solubilized for a specified amount of time and does not significantly degrade or aggregate or become otherwise modified (e.g., as determined, for example, by HPLC). In some embodiments, about 70% or greater, about 80% or greater or about 90% or greater of the compound remains solubilized after the specified period. Stability can also refer to the compatibility of pharmaceutically acceptable excipients described herein. Accordingly, a dosage form can be considered stable when the combined pharmaceutically acceptable excipients and active agent(s) described herein do not degrade or otherwise modify (e.g., react with) the effectiveness or therapeutic value of an active agent described herein.

As used herein, and unless otherwise specified, the term “stable,” when used in connection with a solid formulation or a dosage form, means that the active ingredient of the formulation or dosage form does not significantly degrade, decompose or become otherwise modified (e.g., as determined, for example, by HPLC). In some embodiments, about 85% or greater, about 90% or greater, about 95% or greater or about 98% or greater of the active ingredient remains unchanged after the specified period. Stability can also refer to the compatibility of pharmaceutically acceptable excipients described herein. Accordingly, a dosage form can be considered stable when the combined pharmaceutically acceptable excipients and active agent(s) described herein do not degrade or otherwise modify (e.g., react with) the effectiveness or therapeutic value of an active agent described herein.

As used herein, “administer” or “administration” refers to the act of physically delivering a substance as it exists outside the body into a subject. Administration includes all forms known in the art for delivering therapeutic agents, including but not limited to topical, mucosal, injections, intradermal, intravenous, intramuscular delivery or other method of physical delivery described herein or known in the art (e.g., implantation of a slow-release device, such as a mini-osmotic pump to a subject; liposomal formulations; buccal; sublingual; palatal; gingival; nasal; vaginal; rectal; intra-arteriole; intraperitoneal; intraventricular; intracranial; or transdermal).

“Anti-cancer agents” refer to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxorubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent®, sunitinib malate, and Bevacizumab) or any other cytotoxic agents (estramustine phosphate, prednimustine), hormones or hormone agonists, antagonists, partial agonists or partial antagonists, kinase inhibitors, checkpoint inhibitors, and radiation treatment.

By “co-administer” it is meant that compounds, compositions or agents described herein are administered at the same time, just prior to, or just after the administration of one or more additional compounds, compositions or agents, including for example an anti-cancer agent. Co-administration is meant to include simultaneous or sequential administration of compounds, compositions or agents individually or in combination (more than one compound or agent). Co-administration includes administering two compounds, compositions or agents simultaneously, approximately simultaneously (e.g., within about 1, 5, 10, 15, 20, or 30 minutes of each other), or sequentially in any order. Thus, co-administration can include administering one active agent (e.g. a compound described herein) within 0.5, 1, 2, 4, 6, 8, 10, 12, 16, 20, or 24 hours of a second active agent. Co-administration can also be accomplished by co-formulation, e.g., preparing a single dosage form including both active agents. The active agents can be formulated separately. In such instances, the active agents are admixed and included together in the final form of the dosage unit. Alternatively, co-administration as described herein can include administering two separate unit dosage forms of at least two separate active agents (e.g., Compound 1 and a second active agent described herein).

As used herein, the term “daily” is intended to mean that a therapeutic compound, such as Compound 1, is administered once or more than once each day for a period of time. The term “continuous” is intended to mean that a therapeutic compound, such as Compound 1, is administered daily for an uninterrupted period of at least 10 days to 52 weeks. The term “intermittent” or “intermittently” as used herein is intended to mean stopping and starting at either regular or irregular intervals. For example, intermittent administration of Compound 1 is administration for one to six days per week, administration in cycles (e.g., daily administration for one to ten consecutive days of a 28 day cycle, then a rest period with no administration for rest of the 28 day cycle or daily administration for two to eight consecutive weeks, then a rest period with no administration for up to one week), or administration on alternate days. The term “cycling” as used herein is intended to mean that a therapeutic compound, such as Compound 1, is administered daily or continuously but with a rest period.

A “cycling therapy” refers to a regimen or therapy that includes an administration period as described herein and a rest period as described herein.

The term “administration period” as used herein refers to a period of time a subject is continuously or actively administered a compound or composition described herein.

The term “rest period” as used herein refers to a period of time, often following an administration period, where a subject is not administered a compound or composition described herein (e.g. discontinuation of treatment). In certain embodiments, a “rest period” refers to a period of time where a single agent is not administered to a subject or treatment using a particular compound is discontinued. In such embodiments, a second therapeutic agent (e.g., a different agent than the compound or composition administered in the previous administration period) can be administered to the subject.

An “effective amount” is an amount sufficient to achieve the effect for which it is administered (e.g., treat a disease or reduce one or more symptoms of a disease or condition). Thus, administration of an “amount” of a compound described herein to a subject refers to administration of “an amount effective,” to achieve the desired therapeutic result. A “therapeutically effective amount” of a compound described herein for purposes herein is thus determined by such considerations as are known in the art. The term “therapeutically effective amount” of a composition described herein refers to the amount of the composition that, when administered, is sufficient to treat one or more of the symptoms of a disease described herein (e.g., cancer, for example AML, MDS, MPN or solid tumors). Administration of a compound described herein can be determined according to factors such as, for example, the disease state, age, sex, and weight of the individual. A therapeutically effective amount also refers to any toxic or detrimental effects of Compound 1 are outweighed by the therapeutically beneficial effects.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of a compound provided herein, with or without other additional active agent, after the onset of symptoms of the particular disease. In one embodiment, the disease is leukemia, including, but not limited to, chronic lymphocytic leukemia (CLL), chronic myelocytic leukemia (CML), acute lymphoblastic leukemia (ALL), acute myeloid leukemia or acute myeloblastic leukemia (AML). In one embodiment, the leukemia can be relapsed, refractory or resistant to at least one anti-cancer therapy. In one embodiment, the disease is AML, including, a subtype of AML discussed herein. In one embodiment, the disease is myelodysplastic syndrome MDS, including, a subtype of MDS discussed herein.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.” In one embodiment, the disease is leukemia, including, but is not limited to, chronic lymphocytic leukemia, chronic myelocytic leukemia, acute lymphoblastic leukemia, acute myeloid leukemia, and acute myeloblastic leukemia. In one embodiment, the leukemia can be relapsed, refractory or resistant to at least one anti-cancer therapy. In one embodiment, the disease is AML, including, a subtype of AML discussed herein. In one embodiment, the disease is MDS, including, a subtype of MDS discussed herein.

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease, or lengthening the time during which the remains in remission. In one embodiment, the disease is leukemia, including, but not limited to, chronic lymphocytic leukemia, chronic myelocytic leukemia, acute lymphoblastic leukemia, acute myeloid leukemia, and acute myeloblastic leukemia. In one embodiment, the leukemia can be relapsed, refractory or resistant to at least one anti-cancer therapy. In one embodiment, the disease is AML, including, a subtype of AML discussed herein. In one embodiment, the disease is MDS, including a subtype of MDS discussed herein.

As used herein, “induction therapy” refers to the first treatment given for a disease, or the first treatment given with the intent of inducing complete remission in a disease, such as cancer. When used by itself, induction therapy is the one accepted as the best available treatment. For example, induction therapy for AML comprises treatment with cytarabine for 7 days plus treatment with an anthracycline, such as daunorubicin or idarubicin, for 3 days. If residual leukemia is detected, patients are treated with another chemotherapy course, termed reinduction. If the patient is in complete remission after induction therapy, then additional consolidation and/or maintenance therapy is given to prolong remission or to potentially cure the patient.

As used herein, “consolidation therapy” refers to the treatment given for a disease after remission is first achieved. For example, consolidation therapy for cancer is the treatment given after the cancer has disappeared after initial therapy. Consolidation therapy may include radiation therapy, stem cell transplant, or treatment with cancer drug therapy. Consolidation therapy is also referred to as intensification therapy and post-remission therapy.

As used herein, “maintenance therapy” refers to the treatment given for a disease after remission or best response is achieved, in order to prevent or delay relapse. Maintenance therapy can include chemotherapy, hormone therapy or targeted therapy.

“Remission” as used herein, is a decrease in or disappearance of signs and symptoms of a cancer, for example, multiple myeloma. In partial remission, some, but not all, signs and symptoms of the cancer have disappeared. In complete remission, all signs and symptoms of the cancer have disappeared, although the cancer still may be in the body.

The terms “subject,” “patient,” “subject in need thereof,” and “patient in need thereof” are herein used interchangeably and refer to a living organism suffering from one or more of the diseases described herein (e.g., AML) that can be treated by administration of a composition described herein. Non-limiting examples of organisms include humans, other mammals, bovines, rats, mice, dogs, monkeys, goat, sheep, cows, deer, and other non-mammalian animals. In embodiments, a subject is human. A human subject can be between the ages of about 1 year old to about 100 years old. In embodiments, subjects herein can be characterized by the disease being treated (e.g., a “AML subject”, a “cancer subject”, or a “leukemia subject”).

As used herein, the term “tumor,” refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

As used herein, “hematologic malignancy” refers to cancer of the body's blood-forming and immune system—the bone marrow and lymphatic tissue. Such cancers include leukemias, lymphomas (Non-Hodgkin's Lymphoma), Hodgkin's disease (also called Hodgkin's Lymphoma) and myeloma. In one embodiment, the myeloma is multiple myeloma. In some embodiments, the leukemia is, for example, acute myelogenous leukemia (AML), acute lymphocytic leukemia (ALL), adult T-cell leukemia, chronic lymphocytic leukemia (CLL), hairy cell leukemia, myelodysplasia, myeloproliferative disorders or myeloproliferative neoplasm (MPN), chronic myelogenous leukemia (CML), myelodysplastic syndrome (MDS), human lymphotropic virus-type 1 (HTLV 1) leukemia, mastocytosis, or B-cell acute lymphoblastic leukemia. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin lymphoma (HL), non-Hodgkin lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma. In one embodiment, the hematological malignancy is AML. In another embodiment, the hematological malignancy is MDS.

The term “leukemia” refers to malignant neoplasms of the blood-forming tissues. The leukemia includes, but is not limited to, chronic lymphocytic leukemia, chronic myelocytic leukemia, acute lymphoblastic leukemia, acute myeloid leukemia, and acute myeloblastic leukemia. The leukemia can be relapsed, refractory or resistant to at least one anti-cancer therapy.

In one embodiment, the subject has AML, including, for example, the following subtypes of AML. The term “acute myelogenous or myeloid leukemia” refers to hematological conditions characterized by proliferation and accumulation of primarily undifferentiated or minimally differentiated myeloid cells in the bone marrow, and includes subtypes categorized by either the FAB (French, American, British) or WHO classification system. As described herein, the AML includes the following subtypes based on the FAB classification: MO (AML minimally differentiated); M1 (AML with minimal maturation); M2 (AML with maturation); M3 (Acute promyelocytic leukemia); M4 (Acute myelomonocytic leukemia); M4 (eosAcute myelomonocytic leukemia with eosinophilia); M5 (Acute monocytic leukemia); M6 (Acute erythroid leukemia); and M7 (Acute megakaryoblastic leukemia). As described herein, the AML includes the following subtypes based on the WHO classification: AML with recurrent genetic abnormalities (AML with translocation between chromosomes 8 and 21); AML with translocation or inversion in chromosome 16; AML with translocation between chromosomes 9 and 11; APL (M3) with translocation between chromosomes 15 and 17; AML with translocation between chromosomes 6 and 9; AML with translocation or inversion in chromosome 3); AML (megakaryoblastic) with a translocation between chromosomes 1 and 22; AML with myelodysplasia-related changes; AML related to previous chemotherapy or radiation (Alkylating agent-related AML; Topoisomerase II inhibitor-related AML); AML not otherwise categorized (AML that does not fall into the above categories, i. e. AML minimally differentiated (MO); AML with minimal maturation (M1); AML with maturation (M2); Acute myelomonocytic leukemia (M4); Acute monocytic leukemia (M5); Acute erythroid leukemia (M6); Acute megakaryoblastic leukemia (M7); Acute basophilic leukemia; Acute panmyelosis with fibrosis); Myeloid Sarcoma (also known as granulocytic sarcoma, chloroma or extramedullary myeloblastoma); and Undifferentiated and biphenotypic acute leukemias (also known as mixed phenotype acute leukemias). (see https://www.cancer.org/cancer/acute-myelo id-leukemia/detection-diagnosis-staging/how-classified.html, last accessed May 25, 2017).

In certain embodiments, the risk groups for AML based on cytogenetics are as described below:

| Risk Status | Cytogenetics | Molecular Abnormalities^{a} |
|---|---|---|
| Favorable-risk | Core binding factor: inv(16)^{b,c,d} or | Normal cytogenetics: |
| | t(16;16)^{b,c,d} or 48;21)^{b,d} or | NPM1 mutation in the absence of |
| | t(15;17)^{d} | FLT3-ITD or isolated biallelic |
| | | CEBPA mutation |
| Intermediate- | Normal cytogenetics | Core binding factor with c-KIT |
| risk | +8 alone | mutation^{b} |
| | t(9;11) | |
| | Other non-defined | |
| Poor-risk | Complex (≥3 clonal chromosomal | Normal cytogenetics: |
| | abnormalities) with | FLT3-ITD mutation ^{f} |
| | Monosomal karyotype | TP53 mutation |
| | −5, 5q-, −7, 7q- | |
| | 11q23-non t(9;11) | |
| | inv(3), t(3,3) | |
| | t(6,9) | |
| | t(9;22)^{e} | |

^{a}The molecular abnormalities included in this table reflect those for which validated assays are available in standardized commercial laboratories.
^{b}Emerging data indicate that the presence of KIT mutations in patients with t(8;21), and to a lesser extent inv(16), confers a higher risk of relapse. These patients are considered intermediate risk and should be considered for hematopoietic stem cell transplant (HSCT) or clinical trials, if available. Other cytogenetic abnormalities in addition to these finding do not alter risk status.
^{c}Paschka P, et al. Blood 2013; 121:170-177.
^{d}Other cytogenetic abnormalities in addition to these findings do not alter better risk status
^{e}For Philadelphia + acute myeloid leukemia (AML) t(9;22), manage as myeloid blast crisis in chronic myeloid leukemia (CML), with addition of tyrosine kinase inhibitors.

In one embodiment, the subject has MDS, including, for example, the following subtypes of MDS. The term “myelodysplastic syndrome” refers to hematological conditions characterized by abnormalities in the production of one or more of the cellular components of blood (red cells, white cells (other than lymphocytes) and platelets (or their progenitor cells, megakaryocytes)). The ineffective hematopoiesis in the bone marrow (BM) and peripheral blood cytopenias in MDS manifest clinically as anemia, neutropenia, and/or thrombocytopenia of variable frequency and severity. Anemia is the most frequent laboratory finding and it often progresses to red blood cell (RBC) transfusion dependence. Other less common presenting clinical features related to the cytopenias are an increased risk of infection and/or hemorrhage and a propensity to progress to acute myeloid leukemia (AML) (Catenacci, et al. Blood Rev 2005; 19:301-319).

MDS includes the following disorders: refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms and chronic myelomonocytic leukemia (CMML). The MDS as used herein also includes very low risk, low risk, intermediate risk, high risk and very high risk MDS. In some embodiments, the MDS is primary or de novo MDS. In other embodiments, the MDS is secondary.

In certain embodiments, MDS is classified based on the World Health Organization (WHO) classification of MDS as described below:

WHO classifications for MDS

| WHO myeloid neoplasm | | | |
|---|---|---|---|
| and acute leukemia | Dysplastic | | PB and BM findings and |
| classification | findings | Cytopenias^{a} | cytogenetics |
| MDS with single lineage | 1 | 1 or 2 | BM < 5%, PB < 1%, no Auer |
| dysplasia (MDS-SLD) | | | Rods |
| | | | Any cytogenetics, unless |
| | | | fulfills all criteria for MDS |
| | | | with isolated del(5q) |
| MDS with ring sideroblasts | 1 | 1 or 2 | BM < 5%, PB < 1%, no Auer |
| (MDS-RS)^{b} | 2 or 3 | 3 | Rods |
| MDS-RS and single lineage | | | Any cytogenetics, unless |
| dysplasia | | | fulfills all criteria for MDS |
| MDS-RS and multilineage | | | with isolated del(5q) |
| dysplasia | | | |
| MDS with multilineage | 2 or 3 | 1-3 | BM < 5%, PB < 1%, no Auer |
| dysplasia (MDS-MLD) | | | Rods |
| | | | Any cytogenetics, unless |
| | | | fulfills all criteria for MDS |
| | | | with isolated del(5q) |
| MDS with excess blasts | | | |
| (MDS-EB) | | | |
| MDS-EB-1 | 0-3 | 1-3 | BM 5-9% or PB 2-4%, no |
| | | | Auer Rods |
| | | | Any cytogenetics |
| MDS-EB-2 | 0-3 | 1-3 | BM 10-19% or PB 5-19% |
| | | | or Auer Rods |
| | | | Any cytogenetics |
| MDS with isolated del(5q) | 1-3 | 1-2 | BM < 5%, PB < 1%, no Auer |
| | | | Rods |
| | | | del(5q) alone or with 1 |
| | | | additional abnormality |
| | | | except −7 or del(7q) |
| MDS, unclassifiable (MDS- | | | |
| U) | | | |
| MDS-U with 1% blood | 1-3 | 1-3 | BM < 5%, PB = 1%^{c}, no |
| blasts | | | Auer Rods |
| | | | Any cytogenetics |
| MDS-U with SLD and | 1 | 3 | BM < 5%, PB < 1%, no Auer |
| pancytopenia | | | Rods |
| | | | Any cytogenetics |
| MDS-U based on defining | 0 | 1-3 | BM < 5%, PB < 1%, no Auer |
| cytogenetic abnormality | | | Rods |
| | | | MDS-defining abnormality^{d} |

^{a}Cytopenias defined as: hemoglobin, < 10 g/dL, platelet count, < 100 × 10⁹/L; and absolute neutrophil count, < 1.8 × 10⁹/L. Rarely, MDS may present with mild anemia or thrombocytopenia above these levels. Peripheral blood monocytes must be < 1 × 10⁹/L.
^{b}Cases with ≥ 15% ring sideroblasts by definition have significant erythroid dysplasia, and are classified as MDS-RS-SLD.
^{c}One percent PB blasts must be recorded on at least 2 separate occasions.
^{d}Abnormality must be demonstrated by conventional karyotyping, not by FISH or sequencing. The presence of +8, −Y, of del(20q) is not considered to be MDS-defining in the absence of diagnostic morphologic features of MDS. Arber, et al. Blood 2016;127(20):2391-2405, and Vardiman, et al. Blood. 2009; 114(5):937-51.

As used herein, “promyelocytic leukemia” or “acute promyelocytic leukemia” refers to a malignancy of the bone marrow in which there is a deficiency of mature blood cells in the myeloid line of cells and an excess of immature cells called promyelocytes. It is usually marked by an exchange of regions of chromosomes 15 and 17.

As used herein, “acute lymphocytic leukemia (ALL)”, also known as “acute lymphoblastic leukemia” refers to a malignant disease caused by the abnormal growth and development of early nongranular white blood cells, or lymphocytes.

As used herein, “T-cell leukemia” refers to a disease in which certain cells of the lymphoid system called T lymphocytes or T cells are malignant. T cells are white blood cells that normally can attack virus-infected cells, foreign cells, and cancer cells and produce substances that regulate the immune response.

The term “relapsed” refers to a situation where patients who have had a remission of leukemia after therapy have a return of leukemia cells in the marrow and a decrease in normal blood cells.

The term “refractory or resistant” refers to a circumstance where patients, even after intensive treatment, have residual leukemia cells in their marrow.

The term “drug resistance” refers to the condition when a disease does not respond to the treatment of a certain drug or drugs. Drug resistance can be either intrinsic, which means the disease has never been responsive to the particular drug or drugs, or it can be acquired, which means the disease ceases responding to particular a drug or drugs that the disease had previously responded to. In certain embodiments, drug resistance is intrinsic. In certain embodiments, the drug resistance is acquired.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, ECOG status refers to Eastern Cooperative Oncology Group (ECOG) Performance Status (Oken M, et al Toxicity and response criteria of the Eastern Cooperative Oncology Group. Am J Clin Oncol 1982; 5(6):649-655), as shown below:

| Score | Description |
|---|---|
| 0 | Fully active, able to carry on all pre-disease performance without restriction |
| 1 | Restricted in physically strenuous activity but ambulatory and able to carry out |
| | work of a light or sedentary nature, eg, light housework, office work. |
| 2 | Ambulatory and capable of all self-care but unable to carry out any work |
| | activities. Up and about more than 50% of waking hours. |
| 3 | Capable of only limited self-care, confined to bed or chair more than 50% of |
| | waking hours. |
| 4 | Completely disabled. Cannot carry on any self-care. Totally confined to bed |
| | or chair |
| 5 | Dead |

In the context of a cancer, treatment or inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP as used herein means the time from treatment onset until tumor progression; TTP does not include deaths. Time to Remission (TTR) as used herein means the time from treatment onset until remisison, for example, complete or partial remission. As used herein, PFS means the time from treatment onset until tumor progression or death. In one embodiment, PFS rates will be computed using the Kaplan-Meier estimates. Event-free survival (EFS) means the time from study entry until any treatment failure, including disease progression, treatment discontinuation for any reason, or death. Relapse-free survival (RFS) means the length of time after the treatment ends that the patient survives without any signs or symptoms of that cancer. Overall response rate (ORR) means the sum of the percentage of patients who achieve complete and partial responses. Complete remission rate (CRR) refers to the percentage of patients achieving complete remission (CR). Duration of response (DoR) is the time from achieving a response until relapse or disease progression. Duration of remission is the time from achieving remission, for example, complete or partial remission, until relapse. In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

For leukemia, in particular AML, response to treatment can be assessed based on the International Working Group Response Criteria in AML (Cheson et al. J Clin Oncol 2003; 21(24):4642-9).

Hematologic Response According to IWG Criteria for AML:

| | | | | Bone | |
|---|---|---|---|---|---|
| Response | Time of | Neutrophils | Platelets | Marrow | |
| Criterion | Assessment | (μL) | (μL) | Blasts (%) | Other |
| Early Treatment | 7-10 days | NA | NA | <5 | |
| assessment | after therapy | | | | |
| Morphologic | Varies by | NA | NA | <5 | Flow |
| Leukemia-free | protocol | | | | cytometry |
| State | | | | | EMD |
| Morphologic | Varies by | ≥1,000 | ≥100,000 | <5 | Transfusion |
| CR | protocol | | | | EMD |
| Cytogenetic CR | Varies by | ≥1,000 | ≥100,000 | <5 | Cytogenetics- |
| (CRc) | protocol | | | | normal, |
| | | | | | EMD |
| Molecular CR | Varies by | ≥1,000 | ≥100,000 | <5 | Molecular- |
| (CRm) | protocol | | | | negative, EMD |
| Morphologic | Varies by | Fulfill all cnteria for CR except for residual neutropenia | | | |
| CR with | protocol | (<1,000/μL) or thrombocytopenia (<100,000/μL). | | | |
| incomplete | | | | | |
| blood recovery | | | | | |
| (CRi) | | | | | |
| Partial | Varies by | ≥1,000 | ≥100,000 | Decrease ≥ 50 | Blasts ≤ 5% if |
| Remission | protocol | | | resulting in 5 | Auer rod |
| | | | | to 25 | positive |

| | | |
|---|---|---|
| Relapse after | Varies by | Reappearance of leukemic blasts in the peripheral blood |
| CR | protocol | or ≥ 5% blasts in the bone marrow not attributable to any |
| | | other cause (eg, bone marrow regeneration after |
| | | consolidation therapy). |

Key:
CR = complete remission;
EMD = extramedullary disease;
IWG = International Working Group;
NA = not applicable.

The treatment of lymphoma may be assessed by the International Workshop Criteria (IWC) for NHL (see Cheson B D, et al. J. Clin. Oncol: 2007: (25) 579-586), using the response and endpoint definitions shown below:

| Response | Definition | Nodal Masses | Spleen, liver | Bone Marrow |
|---|---|---|---|---|
| CR | Disappearance | (a) FDG-avid or PET | Not palpable, | Infiltrate cleared on |
| | of all evidence | positive prior to therapy; | nodules | repeat biopsy; if |
| | of disease | mass of any size permitted | disappeared | indeterminate by |
| | | if PET negative | | morphology, |
| | | (b) Variably FDG-avid or | | immunohistochemistry |
| | | PET negative; regression to | | should be negative |
| | | normal size on CT | | |
| PR | Regression of | ≥50% decrease in SPD of | ≥50% | Irrelevant if positive |
| | measurable | up to 6 largest dominant | decrease in | prior to therapy; cell |
| | disease and no | masses; no increase in size | SPD of | type should be |
| | new sites | of other nodes | nodules (for | specified |
| | | (a) FDG-avid or PET | single nodule | |
| | | positive prior to therapy; | in greatest | |
| | | one or more PET positive | transverse | |
| | | at previously involved site | diameter); no | |
| | | (b) Variably FDG-avid or | increase in | |
| | | PET negative; regression | size of liver | |
| | | on CT | or spleen | |
| SD | Failure to | (a) FDG-avid or PET | | |
| | attain CR/PR | positive prior to therapy; | | |
| | or PD | PET positive at prior sites | | |
| | | of disease and no new sites | | |
| | | on CT or PET | | |
| | | (b) Variably FDG-avid or | | |
| | | PET negative; no change in | | |
| | | size of previous lesions on | | |
| | | CT | | |
| PD or | Any new | Appearance of a new | ≥50% | New or recurrent |
| relapsed | lesion or | lesion(s) ≥ 1.5 cm in any | increase from | involvement |
| disease | increase | axis, ≥50% increase in SPD | nadir in the | |
| | by ≥ 50% of | of more than one node, | SPD of any | |
| | previously | or ≥ 50% increase in longest | previous | |
| | involved sites | diameter of a | lesions | |
| | from nadir | previously identifed | | |
| | | node ≥ 1 cm in short axis | | |
| | | Lesions PET positive if | | |
| | | FDG-avid lymphoma or | | |
| | | PET positive prior to | | |
| | | therapy | | |

Abbreviations:
CR, complete remission;
FDG, [¹⁸F]fluorodeoxyglucose;
PET, positron emission tomography;
CT, computed tomography;
PR, partial remission;
SPD, sum of the product of the diameters;
SD, stable disease;
PD, progressive disease.

| | | | Measured |
|---|---|---|---|
| End point | Patients | Definition | from |
| Primary | | | |
| Overall survival | All | Death as a result of any cause | Entry onto |
| | | | study |
| Progression- | All | Disease progression or death as a result of | Entry onto |
| free survival | | any cause | study |
| Secondary | | | |
| Event-free | All | Failure of treatment or death as result of | Entry onto |
| survival | | any cause | study |
| Time to | All | Time to progression or death as a result of | Entry onto |
| progression | | lymphoma | study |
| Disease-free | In CR | Time to relapse or death as a result of | Documentation |
| survival | | lymphoma or acute toxicity of treatment | of response |
| Response | In CR or | Time to relapse or progression | Documentation |
| duration | PR | | of response |
| Lymphoma- | All | Time to death as a result of lymphoma | Entry onto |
| specific survival | | | study |
| Time to next | All | Time to new treatment | End of primary |
| treatment | | | treatment |

Abbreviations:
CR: complete remission;
PR: partial remission.

In one embodiment, the end point for lymphoma is evidence of clinical benefit. Clinical benefit may reflect improvement in quality of life, or reduction in patient symptoms, transfusion requirements, frequent infections, or other parameters. Time to reappearance or progression of lymphoma-related symptoms can also be used in this end point.

The treatment of CLL may be assessed by the International Workshop Guidelines for CLL (see Hallek M, et al. Blood, 2008; (111) 12: 5446-5456) using the response and endpoint definitions shown therein and in particular:

| Parameter | CR | | PR | PD |
|---|---|---|---|---|
| Group A | | | | |
| Lymphadeno- | None > 1.5 cm | | Decrease ≥ 50% | Increase ≥ 50% |
| pathy^{†} | | | | |
| Hepatomegaly | None | | Decrease ≥ 50% | Increase ≥ 50% |
| Splenomegaly | None | | Decrease ≥ 50% | Increase ≥ 50% |
| Blood | <4000/μL | | Decrease ≥ 50% | Increase ≥ 50% over |
| lymphocytes | | | from baseline | baseline |
| Marrow^{‡} | Normocellular, | | 50% reduction in | |
| | <30% | | marrow infiltrate, or | |
| | lymphocytes, | | B-lymphoid nodules | |
| | no B-lymphoid | | | |
| | nodules. | | | |
| | Hypocellular | | | |
| | marrow | | | |
| | defines CRi | | | |
| | (5.1.6). | | | |
| Group B | | | | |
| Platelet count | >100 000 | μL | >100 000 μL or | Decrease of ≥ 50% from |
| | | | increase ≥ 50% over | baseline secondary to |
| | | | baseline | CLL |
| Hemoglobin | >11.0 | g/dL | >11 g/dL or increase | Decrease of > 2 g/dL |
| | | | ≥50% over baseline | from baseline secondary |
| | | | | to CLL |
| Neutrophils^{‡} | >1500 | μL | >1500 μL or > 50% | |

improvement over
baseline

Group A criteria define the tumor load; Group B criteria define the function of the hematopoietic system (or marrow). CR (complete remission): all of the criteria have to be met, and patients have to lack disease-related constitutional symptoms; PR (partial remission): at least two of the criteria of group A plus one of the criteria of group B have to be met; SD is absence of progressive disease (PD) and failure to achieve at least a PR; PD: at least one of the above criteria of group A or group B has to be met. Sum of the products of multiple lymph nodes (as evaluated by CT scans in clinical trials, or by physical examination in general practice). These parameters are irrelevant for some response categories.

The treatment of MM may be assessed by the International Uniform Response Criteria for Multiple Myeloma (IURC) (see Durie et al. Leukemia, 2006; (10) 10: 1-7), using the response and endpoint definitions shown below:

| Response | |
|---|---|
| Subcategory | Response Criteria^{a} |
| sCR | CR as defined below plus |
| | Normal FLC ratio and |
| | Absence of clonal cells in bone marrow^{b} by immunohistochemistry |
| | or immunofluorescence^{c} |
| CR | Negative immunofixation on the serum and urine and |
| | Disappearance of any soft tissue plasmacytomas and < 5% |
| | plasma cells in bone marrow^{b} |
| VGPR | Serum and urine M-protein detectable by immunofixation but not on |
| | electrophoresis or 90% or greater reduction in serum M-protein plus |
| | urine M-protein level < 100 mg per 24 h |
| PR | ≥50% reduction of serum M-protein and reduction in 24-h urinary |
| | M-protein by ≥ 90% or to < 200 mg per 24 h |
| | If the serum and urine M-protein are unmeasurable,^{d} a ≥ 50% |
| | decrease in the difference between involved and uninvolved FLC |
| | levels is required in place of the M-protein criteria |
| | If serum and urine M-protein are unmeasurable, and serum free light |
| | assay is also unmeasurable, ≥50% reduction in plasma cells is |
| | required in place of M-protein, provided baseline bone marrow |
| | plasma cell percentage was ≥ 30% |
| | In addition to the above listed criteria, if present at baseline, a ≥ 50% |
| | reduction in the size of soft tissue plasmacytomas is also required |
| SD (not | Not meeting criteria for CR, VGPR, PR or progressive disease |
| recommended for | |
| use as an indicator of | |
| response; stability of | |
| disease is best | |
| described by | |
| providing the time to | |
| progression | |
| estimates) | |

Abbreviations:
CR, complete response;
FLC, free light chain;
PR, partial response;
SD, stable disease;
sCR, stringent complete response;
VGPR, very good partial response;
^{a}All response categories require two consecutive assessments made at anytime before the institution of any new therapy; all categories also require no known evidence of progressive or new bone lesions if radiographic studies were performed. Radiographic studies are not required to satisfy these response requirements;
^{b}Confirmation with repeat bone marrow biopsy not needed;
^{c}Presence/absence of clonal cells is based upon the κ/λ ratio. An abnormal κ/λ ratio by immunohistochemistry and/or immunofluorescence requires a minimum of 100 plasma cells for analysis. An abnormal ratio reflecting presence of an abnormal clone is κ/λ of > 4:1 or < 1:2.
^{d}Measurable disease defined by at least one of the following measurements: Bone marrow plasma cells ≥ 30%; Serum M-protein ≥ 1g/dl (≥10 gm/l)[10 g/l]; Urine M-protein ≥ 200 mg/24 h; Serum FLC assay: Involved FLC level ≥ 10 mg/dl (≥100 mg/l); provided serum FLC ratio is abnormal.

The treatment of a cancer may also be assessed by Response Evaluation Criteria in Solid Tumors (RECIST 1.1) (see Thereasse P., et al. J. of the National Cancer Institute; 2000; (92) 205-216 and Eisenhauer et al. European J. Cancer; 2009; (45) 228-247). Overall responses for all possible combinations of tumor responses in target and non-target lesions with our without the appearance of new lesions are as follows:

| Target lesions | Non-target lesions | New lesions | Overall response |
|---|---|---|---|
| CR | CR | No | CR |
| CR | Incomplete response/SD | No | PR |
| PR | Non-PD | No | PR |
| SD | Non-PD | No | SD |
| PD | Any | Yes or no | PD |
| Any | PD | Yes or no | PD |
| Any | Any | Yes | PD |

CR = complete response;
PR = partial response;
SD = stable disease; and
PD = progressive disease.

With respect to the evaluation of target lesions, complete response (CR) is the disappearance of all target lesions, partial response (PR) is at least a 30% decrease in the sum of the longest diameter of target lesions, taking as reference the baseline sum longest diameter, progressive disease (PD) is at least a 20% increase in the sum of the longest diameter of target lesions, taking as reference the smallest sum longest diameter recorded since the treatment started or the appearance of one or more new lesions and stable disease (SD) is neither sufficient shrinkage to qualify for partial response nor sufficient increase to qualify for progressive disease, taking as reference the smallest sum longest diameter since the treatment started.

With respect to the evaluation of non-target lesions, complete response is the disappearance of all non-target lesions and normalization of tumor marker level; incomplete response/stable disease is the persistence of one or more non-target lesion(s) and/or the maintenance of tumor marker level above the normal limits, and progressive disease (PD) is the appearance of one or more new lesions and/or unequivocal progression of existing non-target lesions.

The treatment of MDS may be assessed by International Working Group (IWG) Response Criteria for Myelodysplasia.

Modified IWG Response Criteria for MDS

| Category | Response criteria (responses must last at least 4 weeks) |
|---|---|
| Complete remission (CR) | Bone marrow: ≤5% myeloblasts with normal maturation of all |
| | cell lines^{a} |
| | Persistent dysplasia will be noted^{a,b} |
| | Peripheral blood^{c} |
| | Hemoglobin ≥ 11 g/dL |
| | Platelets ≥ 100 × 10⁹/L |
| | Neutrophils ≥ 1.0 × 10⁹/L^{b} |
| | Blasts 0% |
| Partial remission (PR) | All CR criteria if abnormal before treatment, except: |
| | Bone marrow blasts decreased by ≥50% over pretreatment but |
| | still >5% |
| | Cellularity and morphology not relevant |
| Marrow CR^{b} ± | Bone marrow: ≤5% myeloblasts and decrease by ≥50% over |
| Hematologic | pretreatment^{b} Note: Blasts at baseline must be ≥5% in order for |
| Improvement (HI) | subject to be evaluable for Marrow CR^{d} |
| | Peripheral blood: if HI responses, they will be noted in addition to |
| | marrow CR^{b} |
| Stable disease (SD) | Failure to achieve at least PR, but no evidence of progression for |
| | >8 weeks |
| Failure | Death during treatment or disease progression characterized by |
| | worsening of cytopenias, increase in percentage of bone marrow |
| | blasts, or progression to a more advanced MDS FAB subtype |
| | than pretreatment |
| Relapse after CR or PR | At least 1 of the following: |
| | Return to pretreatment bone marrow blast percentage |
| | Decrement of ≥50% from maximum remission/response |
| | levels in granulocytes or platelets |
| | Reduction of Hgb concentration by ≥1.5 g/dL or transfusion |
| | dependence |
| Cytogenetic Response | Complete-Disappearance of the chromosomal abnormality |
| | without appearance of new ones |
| | Partial-At least 50% reduction of the chromosomal abnormality |
| Disease Progression (PD) | For patients with: |
| | Less than 5% blasts: ≥50% increase in blasts to >5% blasts |
| | 5%-10% blasts: ≥50% increase in blasts to >10% blasts |
| | 10%-20% blasts: ≥50% increase in blasts to >20% blasts |
| | Any of the following: |
| | At least 50% decrement from maximum remission/response |
| | levels in granulocytes or platelets |
| | Reduction in Hgb concentration by ≥2 g/dL |
| | Transfusion dependence |
| Disease transformation | Transformation to AML (20% or more BM or PB blasts)^{d} |
| Hematologic | |
| Improvement (HI) | |
| Erythroid response | Hgb increase by ≥1.5 g/dL |
| (HI-E) | Relevant reduction of units of RBC transfusions by an absolute |
| (Pretreatment < 11 | number of at least 4 RBC transfusions/8 weeks compared with the |
| g/dL) | pretreatment transfusion number in the previous 8 weeks. Only |
| | RBC transfusions given for a Hgb of ≤9.0 g/dL pretreatment will |
| | count in the RBC transfusion evaluation |
| Platelet response (HI-P) | Absolute increase of ≥30 × 10⁹/L for patients starting with >20 × |
| (Pretreatment < 100 × | 10⁹/L |
| 10⁹/L) | Increase from <20 × 10⁹/L to >20 × 10⁹/L and by at least 100% |
| Neutrophil response | At least 100% increase and an absolute increase of >0.5 × 10⁹/L |
| (HI-N) | |
| (Pretreatment < 1.0 × | |
| 10⁹/L) | |
| Progression/relapse | At least one of the following: |
| after HI | At least 50% decrement from maximum response levels in |
| | granulocytes or platelets |
| | Reduction in Hgb by ≥1.5 g/dL |
| | Transfusion dependence |

BM = bone marrow; CR = complete remission; FAB = French-American-British; Hgb = hemoglobin; HI = hematologic improvement; IWG = International Working Group; MDS = myelodysplastic syndromes; PB = peripheral blood; PD = Disease Progression; PR = partial remission; RBC = red blood cell.
^{a}Dysplastic changes should consider the normal range of dysplastic changes (modification).
^{b}Modification to IWG response criteria.
^{c}In some circumstances, protocol therapy may require the initiation of further treatment (eg, consolidation, maintenance) before the 4-week period. Such subjects can be included in the response category into which they fit at the time the therapy is started. Transient cytopenias during repeated chemotherapy courses should not be considered as interrupting durability of response, as long as they recover to the improved counts of the previous course.
^{d}Sponsor modification of IWG criteria.
Sources: Cheson, 2006 and Vardiman, 2008.

RBC and Platelet Transfusion Independence

| | At Screening | During Study Treatment |
|---|---|---|
| RBC transfusion | Subjects who received | Subjects who experienced a |
| independence | <4 RBC units during | Hgb increase of 1.5 g/dL |
| | the previous 56 days | over baseline and who |
| | | received no RBC |
| | | transfusions during a |
| | | 56-day period on treatment. |
| | | Note: Only RBC |
| | | transfusions given |
| | | for a Hgb of ≤9.0 g/dL |
| | | within 3 days prior to the |
| | | transfusion will count in the |
| | | RBC transfusion response |
| | | evaluation |
| RBC transfusion | Subjects who received | |
| dependence | ≥4 RBC units during | |
| | the previous 56 days | |
| Platelet | Subjects who received | Subjects who received no |
| transfusion | <2 platelet transfusions | platelet transfusions during |
| independence | during the previous 56 | a 56-day period on |
| | days | treatment |
| Platelet | Subjects who received | |
| transfusion | ≥2 platelet transfusions | |
| dependence | during the previous 56 | |
| | days. | |

RBC = red blood cell; Hgb = hemoglobin.
^{a} RBC transfusion independence and RBC transfusion dependence are defined according to modified IWG criteria.
^{b}Platelet transfusion independence and platelet transfusion dependence are defined by the Sponsor.
Source: Cheson, et at. Blood. 2006; 108(2): 419-25.

Revised International Prognostic Scoring System is used for prognosis of MDS as follows:

IPSS-R Cytogenetic Risk Group

| Cytogenetic | |
|---|---|
| Prognostic | |
| Subgroups | Cytogenetic Abnormalities |
| Very good | −Y, del(11q) |
| Good | Normal, del(5q), del(12p), del(20q), double including |
| | del(5q) |
| Intermediate | del(7q), +8, +19, i(17q), any other single or double |
| | independent clones |
| Poor | −7, inv(3)/t(3q)/del(3q), double including −7/del(7q), |
| | Complex: 3 abnormalities |
| Very poor | Complex: >3 abnormalities |

Source: Greenburg, et al. Blood. 2012; 120(12): 2454-65.

IPSS-R Prognostic Score Values

| Prognostic | | | | | | | |
|---|---|---|---|---|---|---|---|
| variable | 0 | 0.5 | 1 | 1.5 | 2 | 3 | 4 |
| Cytogenetics | Very | — | Good | — | Inter- | Poor | Very |
| | Good | | | | mediate | | Poor |
| Bone Marrow | ≤2 | — | >2-<5 | — | 5-10 | >10 | — |
| Blast (%) | | | | | | | |
| Hemoglobin (g/dL) | ≥10 | — | 8-<10 | <8 | — | — | — |
| Platelets (×10⁹/L) | ≥100 | 50-<100 | <50 | — | — | — | — |
| ANC (×10⁹/L) | ≥0.8 | <0.8 | — | — | — | — | — |

Source: Greenburg, et al. Blood. 2012;120(12):2454-65.

The total IPSS-R score is calculated as the sum of the cytogenetics, bone marrow blast percentage, hemoglobin, platelets and ANC individual scores.

IPSS-R Prognostic Risk Categories/Scores

| Risk Category | Risk Score |
|---|---|
| Very Low | ≤1.5 |
| Low | >1.5-3 |
| Intermediate | >3-4.5 |
| High | >4.5-6 |
| Very High | >6 |

Source: Greenburg, et al. Blood. 2012; 120(12): 2454-65.

IPSS-R: Prognostic Risk Category Clinical Outcomes

| Prognostic | No. | Very | | | | Very |
|---|---|---|---|---|---|---|
| variable | pts | Low | Low | Intermediate | High | High |
| Patients, % | 7012 | 19% | 38% | 20% | 13% | 10% |
| Median Overall | — | 8.8 | 5.3 | 3.0 | 1.6 | 0.8 |
| Survival (years) | | | | | | |
| Median time to 25% | — | Not | 10.8 | 3.2 | 1.4 | 0.73 |
| AML evolution | | reached | | | | |

Source: Greenberg, et al. Blood 2012;120(12):2454-65

Compound

The compound suitable for use in the methods and formulations provided herein is Compound 1: 2-(4-chlorophenyl)-N-((2-(2,6-dioxopiperidin-3-yl)-1-oxoisoindolin-5-yl)methyl)-2,2-difluoroacetamide having the structure:

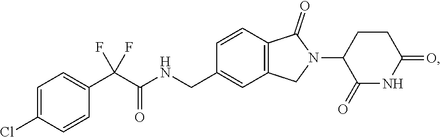 [SMILES: O=C1CCC(N2Cc3cc(CNC(=O)C(F)(F)c4ccc(Cl)cc4)ccc3C2=O)C(=O)N1]

or its stereoisomers or mixture of stereoisomers, isotopologues, pharmaceutically acceptable salts, tautomers, solvates, hydrates, co-crystals, clathrates, or polymorphs thereof. In certain embodiments, Compound 1 refers to 2-(4-chlorophenyl)-N-((2-(2,6-dioxopiperidin-3-yl)-1-oxoisoindolin-5-yl)methyl)-2,2-difluoroacetamide.

Compound 1 can be prepared according to the methods described in the Examples provided herein or as described in U.S. Pat. No. 9,499,514, the disclosure of which is incorporated herein by reference in its entirety. The compound can also be synthesized according to other methods apparent to those of skill in the art based upon the teaching herein.

In certain embodiments, the compound is an isotopologue of Compound 1, as described in U.S. Patent application No. 62/612,926, filed Jan. 2, 2018, which is incorporated herein by reference in its entirety.

In certain embodiments, Compound 1 is a solid. In certain embodiments, Compound 1 is a hydrate. In certain embodiments, Compound 1 is solvated. In certain embodiments, Compound 1 is anhydrous.

In certain embodiments, Compound 1 is amorphous. In certain embodiments, Compound 1 is crystalline. In certain embodiments, Compound 1 is in a crystalline form described in U.S. Pat. No. 10,189,808, which is incorporated herein by reference in its entirety. Exemplary solid forms are described in column nos. 16-23 and 66-70 of U.S. Pat. No. 10,189,808.

The solid forms of Compound 1 can be prepared according to the methods described in the disclosure of U.S. Pat. No. 10,189,808, see column nos. 66-70. The solid forms can also be prepared according to other methods apparent to those of skill in the art.

In one embodiment, Compound 1 is polymorph Form A of 2-(4-chlorophenyl)-N-((2-(2,6-dioxopiperidin-3-yl)-1-oxoisoindolin-5-yl)methyl)-2,2-difluoroacetamide (as described in column nos. 16-17 and 66 of U.S. Pat. No. 10,189,808). In one embodiment, Compound 1 is polymorph Form B of 2-(4-chlorophenyl)-N-((2-(2,6-dioxopiperidin-3-yl)-1-oxoisoindolin-5-yl)methyl)-2,2-difluoroacetamide (as described in column nos. 18-19 and 66-67 of U.S. Pat. No. 10,189,808). In one embodiment, Compound 1 is polymorph Form C of 2-(4-chlorophenyl)-N-((2-(2,6-dioxopiperidin-3-yl)-1-oxoisoindolin-5-yl)methyl)-2,2-difluoroacetamide (as described in column nos. 19-20 and 67-68 of U.S. Pat. No. 10,189,808). In one embodiment, Compound 1 is polymorph Form D of 2-(4-chlorophenyl)-N-((2-(2,6-dioxopiperidin-3-yl)-1-oxoisoindolin-5-yl)methyl)-2,2-difluoroacetamide (as described in column nos. 20-21 and 68-69 of U.S. Pat. No. 10,189,808). In one embodiment, Compound 1 is polymorph Form E of 2-(4-chlorophenyl)-N-((2-(2,6-dioxopiperidin-3-yl)-1-oxoisoindolin-5-yl)methyl)-2,2-difluoroacetamide (as described in column nos. 22-23 and 69-70 of U.S. Pat. No. 10,189,808). In one embodiment, Compound 1 is an amorphous form of 2-(4-chlorophenyl)-N-((2-(2,6-dioxopiperidin-3-yl)-1-oxoisoindolin-5-yl)methyl)-2,2-difluoroacetamide (as described in column nos. 23 and 70 of U.S. Pat. No. 10,189,808).

Formulations of Compound 1

In one aspect, provided herein are stable formulations of Compound 1. In one embodiment, the formulations of Compound 1 comprise a solid form of 2-(4-chlorophenyl)-N-((2-(2,6-dioxopiperidin-3-yl)-1-oxoisoindolin-5-yl)methyl)-2,2-difluoroacetamide. In one embodiment, the formulations of Compound 1 comprise an amorphous form of 2-(4-chlorophenyl)-N-((2-(2,6-dioxopiperidin-3-yl)-1-oxoisoindolin-5-yl)methyl)-2,2-difluoroacetamide. The solid forms of Compound 1 are described in U.S. Pat. No. 10,189,808.

A. Mannitol Formulations

In certain embodiments, the formulation of Compound 1 further comprises mannitol. In certain embodiments, the formulation of Compound 1 further comprises mannitol and a citrate buffer. In certain embodiments, the formulation of Compound 1 is a lyophilized formulation. In certain embodiments, the formulation of Compound 1 is an aqueous formulation. In certain embodiments, the lyophilized formulations provided herein comprise about 1.0% to 1.3% Compound 1, about 9.0% to 12.0% citrate buffer and about 85.0% to 90.0% mannitol based on the total weight of the lyophilized formulation.

In one embodiment, the lyophilized formulations provided herein comprise about 1% Compound 1, about 11% citrate buffer and about 88% mannitol based on the total weight of the lyophilized formulation.

In one embodiment, the lyophilized formulations provided herein comprise about 1.1% Compound 1, about 10.6% citrate buffer and about 88.0% mannitol based on the total weight of the lyophilized formulation.

In one embodiment, the lyophilized formulations provided herein comprise about 1.10% Compound 1, about 10.63% citrate buffer and about 88.00% mannitol based on the total weight of the lyophilized formulation.

In certain embodiments, the lyophilized formulations provided herein comprise about 1.0% to 1.3% Compound 1, about 4.0% to about 7.5% citric acid monohydrate, about 3.0% to 5.5% sodium citrate dihydrate and about 85.0% to 90.0% mannitol based on the total weight of the lyophilized formulation.

In one embodiment, the lyophilized formulations provided herein comprise about 1% Compound 1, about about 6% citric acid monohydrate, about 5% sodium citrate dihydrate and about 88% mannitol based on the total weight of the lyophilized formulation.

In one embodiment, the lyophilized formulations provided herein comprise about 1.1% Compound 1, about 5.8% citric acid monohydrate, about 4.9% sodium citrate dihydrate and about 88.0% mannitol based on the total weight of the lyophilized formulation.

In one embodiment, the lyophilized formulations provided herein comprise about 1.10% Compound 1, about 5.78% citric acid monohydrate, about 4.85% sodium citrate dihydrate and about 88.00% mannitol based on the total weight of the lyophilized formulation.

In one aspect, the lyophilized formulation provided herein comprises Compound 1 in an amount of about 1 to about 1.25% based on the total weight of the lyophilized formulation. In certain embodiments, the amount of Compound 1 is about 1.0%, 1.1% or 1.2% based on the total weight of the lyophilized formulation. In one embodiment, the amount of Compound 1 in the lyophilized formulation is about 1.1% based on the total weight of the lyophilized formulation.

In another aspect is a lyophilized formulation that comprises Compound 1 in an amount of about 0.9 mg to about 1.1 mg in a 20 cc vial. In one aspect Compound 1 is present in an amount of about 0.9, 0.95, 1.0, 1.05 or 1.1 mg in a 20 cc vial. In one aspect Compound 1 is present in an amount of about 1 mg in a 20 cc vial.

In one aspect, the lyophilized formulations provided herein contain a citrate buffer. In one aspect, the amount of citrate buffer in the formulations provided herein is from about 9% to about 11% based on total weight of the lyophilized formulation. In one aspect, the amount of citrate buffer in the formulations provided herein is about 9%, 10%, 11% or 12% based on total weight of the lyophilized formulation. In one aspect, the amount of citrate buffer in the formulations provided herein is about 10.63% based on total weight of the lyophilized formulation.

In one embodiment, the citrate buffer comprises citric acid monohydrate and sodium citrate dihydrate. In certain embodiments, the amount of citric acid monohydrate is from about 4% to about 7.5% or about 5% to about 6% based on total weight of the lyophilized formulation. In certain embodiments, the amount of citric acid monohydrate in the lyophilized formulation is about 5.5%, 5.78%, 6%, 6.2%, or 6.5% based on total weight of the lyophilized formulation. In one embodiment, the amount of citric acid monohydrate in the lyophilized formulation is about 5.78% based on total weight of the lyophilized formulation.

In still another aspect is a lyophilized formulation that comprises citric acid monohydrate in an amount of about 4 mg to about 6.5 mg in a 20 cc vial. In one embodiment, the amount of citric acid monohydrate is about 4.5, 4.75, 5, 5.24, 5.5 or 6 mg in a 20 cc vial. In one embodiment, the amount of citric acid monohydrate is about 5.24 mg in a 20 cc vial.

In certain embodiments, the amount of sodium citrate dihydrate is from about 3% to about 5.5% or about 4% to about 5% based on total weight of the lyophilized formulation. In certain embodiments, the amount of sodium citrate dihydrate in the lyophilized formulation is about 3.5%, 4%, 4.5%, 4.85%, 5% about 5.5% based on total weight of the lyophilized formulation. In one embodiment, the amount of sodium citrate dihydrate in the lyophilized formulation is about 4.85% based on total weight of the lyophilized formulation.

In still another aspect is a lyophilized formulation that comprises sodium citrate dihydrate in an amount of about 3.5 mg to about 5.5 mg in a 20 cc vial. In one embodiment, the amount of sodium citrate dihydrate is about 4, 4.25, 4.4, 4.5, 4.75 or 5 mg in a 20 cc vial. In one embodiment, the amount of sodium citrate dihydrate is about 4.4 mg in a 20 cc vial.

In still another aspect is a lyophilized formulation that comprises mannitol from about 80% to about 95% or about 85% to about 90% based on total weight of the lyophilized formulation. In one embodiment, the amount of mannitol in the lyophilized compositions provided herein is about 80%, 82%, 84%, 86%, 88% or 90% based on total weight of the lyophilized formulation. In one embodiment, the amount of mannitol in the lyophilized compositions provided herein is about 88% based on total weight of the lyophilized formulation.

In another aspect is a lyophilized formulation that comprises mannitol in an amount of about 75, 78, 80, or 82 mg in a 20 cc vial. In still another aspect is a lyophilized formulation that comprises mannitol in an amount of about 80 mg in a 20 cc vial.

In another aspect is a lyophilized formulation that comprises Compound 1 in an amount of about 0.9 mg to about 1.1 mg, citric acid monohydrate in an amount of about 4 mg to about 6.5 mg, sodium citrate dihydrate in an amount of about 3.5 mg to about 5.5 mg, and mannitol in an amount of about 75 to 82 mg in a 20 cc vial.

In another aspect is a lyophilized formulation that comprises Compound 1 in an amount of about 1.0 mg, citric acid monohydrate in an amount of about 5.2 mg, sodium citrate dihydrate in an amount of about 4.4 mg, and mannitol in an amount of about 80.0 mg in a 20 cc vial.

In another aspect is a lyophilized formulation that comprises Compound 1 in an amount of about 1.00 mg, citric acid monohydrate in an amount of about 5.24 mg, sodium citrate dihydrate in an amount of about 4.40 mg, and mannitol in an amount of about 80.00 mg in a 20 cc vial.

In one aspect provided herein is a formulation in a 20 cc vial, that consists essentially of Compound 1 at an amount that provides about 0.9 mg to about 1.1 mg 2-(4-chlorophenyl)-N-((2-(2,6-dioxopiperidin-3-yl)-1-oxoisoindolin-5-yl)methyl)-2,2-difluoroacetamide, about 75 to 82 mg mannitol, about 4 mg to about 6.5 mg citric acid monohydrate and about 3.5 mg to about 5.5 mg sodium citrate dihydrate.

In one aspect provided herein is a formulation in a 20 cc vial that consists essentially of Compound 1 at an amount that provides about 1.0 mg 2-(4-chlorophenyl)-N-((2-(2,6-dioxopiperidin-3-yl)-1-oxoisoindolin-5-yl)methyl)-2,2-difluoroacetamide, about 80.0 mg mannitol, about 5.2 mg citric acid monohydrate and about 4.4 mg sodium citrate dihydrate.

In one aspect provided herein is a formulation in a 20 cc vial that comprises: Compound 1 at an amount that provides about 1 mg 2-(4-chlorophenyl)-N-((2-(2,6-dioxopiperidin-3-yl)-1-oxoisoindolin-5-yl)methyl)-2,2-difluoroacetamide, 80 mg mannitol, 5.24 mg citric acid monohydrate and 4.4 mg sodium citrate dihydrate.

In one embodiment, provided herein is an aqueous formulation comprising Compound 1 in an amount of about 0.9 mg/mL to about 1.1 mg/mL, mannitol in an amount of about 75 mg/mL to 82 mg/mL, citric acid monohydrate in an amount of about 4 mg/mL to about 6.5 mg/mL, and sodium citrate dihydrate in an amount of about 3.5 mg/mL to about 5.5 mg/mL.

In one aspect provided herein is an aqueous formulation comprising Compound 1 in an amount of about 0.1 mg/mL, mannitol in an amount of about 8.0 mg/mL, citric acid monohydrate in an amount of about 0.5 mg/mL and sodium citrate dehydrate in an amount of in an amount of about 0.4 mg/mL.

In one embodiment, provided herein is an aqueous formulation comprising Compound 1 in an amount of about 0.10 mg/mL, mannitol in an amount of about 8.00 mg/mL, citric acid monohydrate in an amount of about 0.52 mg/mL, and sodium citrate dihydrate in an amount of about 0.44 mg/mL.

In one embodiment, provided herein is an aqueous formulation consisting essentially of Compound 1 in an amount of about 0.10 mg/mL, mannitol in an amount of about 8.0 mg/mL, citric acid monohydrate in an amount of about 0.52 mg/mL, and sodium citrate dihydrate in an amount of about 0.44 mg/mL.

In certain embodiments, the formulations provided herein are lyophilized formulations. In certain embodiments, the formulations provided herein are aqueous formulations. In certain embodiments, the formulations provided herein are reconstituted formulations obtained in a pharmaceutically acceptable solvent to produce a pharmaceutically acceptable solution.

In certain embodiments, the formulation upon reconstitution has a pH of about 4 to 5. In one embodiment, the formulation upon reconstitution has a pH of about 4, 4.1, 4.2, 4.3, 4.4, 4.5, 4.6, 4.7, 4.8, 4.9 or 5.

In certain embodiments, provided herein is a container comprising a formulation provided herein. In certain embodiments, provided herein is a container comprising a lyophilized formulation provided herein. In one aspect, the container is a glass vial. In one aspect, the container is a 20 cc glass vial.

In certain embodiments, the vial comprises about 1.0% to 1.3% Compound 1, about 9.0% to 12.0% citrate buffer and about 85.0% to 90.0% mannitol based on the total weight of the formulation in the vial.

In one embodiment, the vial comprises about 1% Compound 1, about 11% citrate buffer and about 88% mannitol based on the total weight of the formulation in the vial.

In one embodiment, the vial comprises about 1.1% Compound 1, about 10.6% citrate buffer and about 88.0% mannitol based on the total weight of the formulation in the vial.

In one embodiment, the vial comprises about 1.10% Compound 1, about 10.63% citrate buffer and about 88.00% mannitol based on the total weight of the formulation in the vial.

In one aspect, the vial comprises about 0.9 mg to about 1.1 mg Compound 1, about 4 mg to about 6.5 mg citric acid monohydrate, about 3.5 mg to about 5.5 mg sodium citrate dihydrate and about 75 to 82 mg mannitol.

In one aspect, the vial comprises about 1.0 mg Compound 1, about 5.2 mg citric acid monohydrate, about 4.4 mg sodium citrate dihydrate and about 80.0 mg mannitol.

In one aspect, the vial comprises 1.00 mg Compound 1, 5.24 mg citric acid monohydrate, 4.40 mg sodium citrate dihydrate and 80.00 mg mannitol.

The lyophilized formulations of Compound 1 provided herein can be administered to a patient in need thereof using standard therapeutic methods for delivering Compound 1 including, but not limited to, the methods described herein. In one embodiment, the lyophilized formulations provided herein are reconstituted in a pharmaceutically acceptable solvent to produce a pharmaceutically acceptable solution, wherein the solution is administered (such as by intravenous injection) to the patient.

The lyophilized formulation provided herein can be reconstituted for parenteral administration to a patient using any pharmaceutically acceptable diluent. Such diluents include, but are not limited to a solution of PEG400, ethanol, and water for injection. In one embodiment, the diluent comprises PEG400, ethanol, and water for injection, for example, in a volume ratio of 50:10:40. In one embodiment, the reconstitution diluent solution has the following composition (10 mL/vial in 20 cc vial):

| Material | Composition (g/mL) | Composition (g/vial)^{b} |
|---|---|---|
| PEG 400 | 0.565 | 5.65 |
| Ethanol | 0.079 | 0.79 |
| Water for Injection (WFI) | 0.400 | 4.00 |

^{b}bulk solution density = 0.898 g/ml

Any quantity of diluent may be used to constitute the lyophilized formulation such that a suitable solution for injection is prepared. Accordingly, the quantity of the diluent must be sufficient to dissolve the lyophilized formulation. In one embodiment, 4-6 mL of a diluent are used to constitute the lyophilized formulation to yield a final concentration of, about 0.1-0.3 mg/mL, about 0.15 mg/mL, or about 0.2 mg/mL of Compound 1. In certain embodiments, the final concentration of Compound 1 in the reconstituted solution is about 0.2 mg/mL. In certain embodiments, depending on the required dose, multiple vials may be used for reconstitution.

The reconstituted solutions of lyophilized formulation can be stored and used within up to about 24 hours, about 12 hours or about 8 hours. In some embodiments, the solution is used within 8 hours of preparation. In some embodiments, the solution is used within 5 hours of preparation. In some embodiments, the solution is used within 1 hour of preparation.

Process for Preparing Mannitol Formulations

The formulations comprising mannitol can be prepared by any of the methods known in the art and as described herein, but all methods include the step of bringing the active ingredient into association with the pharmaceutically acceptable excipient, which constitutes one or more necessary ingredients (such as bulking agent and/or buffer).

In one aspect, the formulations provided herein are prepared by dissolving mannitol in tert-butyl alcohol and citrate buffer to obtain a buffer solution, and dissolving Compound 1 in the buffer solution to a drug solution. In one aspect, the drug solution is lyophilized to obtain a lyophilized formulation.

In one aspect, the formulations provided herein are prepared by dissolving a citrate buffer in water, adding mannitol to the buffer solution, followed by addition of tert-butyl alcohol (tBA). Compound 1 is then added to the tBA/buffer mixture to obtain a solution; and optionally lyophilizing the solution to obtain the lyophilized formulation. The solution of Compound 1 in tBA/buffer mixture is optionally filtered, for example through 0.22 μm PVDF filter.

In one embodiment, the vial is sealed under nitrogen after lyophilization.

In one aspect, the lyophilization process contains three stages: freezing, primary drying, and secondary drying. A liquid formulation is transformed to a lyophilized powder form by going through complete solidification through freezing stage, sublimation of ice and solvents through primary drying, and desorption of residual moisture and solvents through secondary drying. The shelf temperature and chamber pressure in the primary drying and secondary drying are controlled to obtain the desired quality of the finished drug product. In one aspect of the process, the cake appearance and structure are characterized by visual inspection.

B. Human Albumin Formulations

In certain embodiment, the formulations provided herein comprise Compound 1 and human albumin. In certain embodiment, the formulations provided herein comprise Compound 1, human albumin and a citrate buffer. In certain embodiment, the formulations provided herein comprise Compound 1, a citrate buffer, human albumin, and sucrose.

In certain embodiment, the formulations provided herein comprise Compound 1, citric acid anhydrous, sodium citrate dihydrate, human albumin and sucrose.

In certain embodiment, the formulations provided herein comprise Compound 1, citric acid anhydrous, sodium citrate dihydrate, human albumin, sucrose and formic acid. In one embodiment, formic acid is removed during lyopholization.

In certain embodiment, the formulations provided herein comprise Compound 1, citric acid anhydrous, sodium citrate dihydrate, human albumin, sucrose, formic acid and acetic acid.

In certain embodiment, the formulations provided herein comprise Compound 1, citric acid, human albumin and sucrose. In one embodiment, the formulation further comprises sodium chloride. In one embodiment, the formulation further comprises sodium N-acetyltryptophanate. In one embodiment, the formulation further comprises sodium caprylate.

In certain embodiment, the formulations provided herein comprise Compound 1, citric acid, human albumin and trehalose. In one embodiment, the formulation further comprises sodium chloride. In one embodiment, the formulation further comprises sodium N-acetyltryptophanate. In one embodiment, the formulation further comprises sodium caprylate.

In certain embodiment, the formulations provided herein comprise Compound 1, citric acid, human albumin, trehalose and mannitol. In one embodiment, the formulation further comprises sodium chloride. In one embodiment, the formulation further comprises sodium N-acetyltryptophanate. In one embodiment, the formulation further comprises sodium caprylate.

In one embodiment, the formulations provided herein comprise human albumin and Compound 1 in a ratio of at least 500. In one embodiment, the formulations provided herein comprise human albumin and Compound 1 in a ratio of 500 to 2000. In one embodiment, the formulations provided herein comprise human albumin and Compound 1 in a ratio of 500 to 1000. In one embodiment, the formulations provided herein comprise human albumin and Compound 1 in a ratio of 500. In one embodiment, the formulations provided herein comprise human albumin and Compound 1 in a ratio of 1000. In one embodiment, the formulations provided herein comprise human albumin and Compound 1 in a ratio of 1500. In one embodiment, the formulations provided herein comprise human albumin and Compound 1 in a ratio of 2000.

In one embodiment, the formulation provided herein comprises about 0.03% to 0.25% Compound 1, about 30.00% to 90.00% human albumin, about 20.00% to 60.00% sucrose, and about 1.00% to 8.00% citric acid based on the total weight of the formulation. In certain embodiments, the formulation further comprises about 1.00% to 9.00% sodium chloride based on the total weight of the formulation. In certain embodiments, the formulation further comprises about 0.50% to 2.50% sodium N-acetyltryptophanate based on the total weight of the formulation. In certain embodiments, the formulation further comprises about 0.3% to 1.2% sodium caprylate based on the total weight of the formulation.

In one embodiment, the formulation provided herein comprises about 0.03% to 0.25% Compound 1, about 35.00% to 90.00% human albumin, about 25.00% to 60.00% sucrose, and about 1.00% to 8.00% citric acid based on the total weight of the formulation. In certain embodiments, the formulation further comprises about 1.00% to 9.00% sodium chloride based on the total weight of the formulation. In certain embodiments, the formulation further comprises about 0.50% to 2.50% sodium N-acetyltryptophanate based on the total weight of the formulation. In certain embodiments, the formulation further comprises about 0.30% to 1.2% sodium caprylate based on the total weight of the formulation.

In one embodiment, the formulation provided herein comprises about 0.03% to 0.06% Compound 1, about 35.00% to 50.00% human albumin, about 40.00% to 60.00% sucrose, and about 2.50% to 4.50% citric acid based on the total weight of the formulation. In certain embodiments, the formulation further comprises about 1.00% to 3.00% sodium chloride based on the total weight of the formulation. In certain embodiments, the formulation further comprises about 0.50% to 1.50% sodium N-acetyltryptophanate based on the total weight of the formulation. In certain embodiments, the formulation further comprises about 0.30% to 0.70% sodium caprylate based on the total weight of the formulation.

In one embodiment, the formulation provided herein comprises about 0.03% to 0.05% Compound 1, about 38.00% to 47.00% human albumin, about 45.00% to 55.00% sucrose, and about 30.00% to 40.00% citric acid based on the total weight of the formulation. In certain embodiments, the formulation further comprises about 1.50% to 2.50% sodium chloride based on the total weight of the formulation. In certain embodiments, the formulation further comprises about 0.75% to 1.25% sodium N-acetyltryptophanate based on the total weight of the formulation. In certain embodiments, the formulation further comprises about 0.45% to 0.65% sodium caprylate based on the total weight of the formulation.

In one embodiment, the formulation provided herein comprises about 0.05% to 0.15% Compound 1, about 35.00% to 60.00% human albumin, about 10.00% to 60.00% sucrose, about 2.00% to 5.00% citric acid based on the total weight of the formulation. In certain embodiments, the formulation further comprises about 1.00% to 3.00% sodium chloride based on the total weight of the formulation. In certain embodiments, the formulation further comprises about 0.50% to 2.50% sodium N-acetyltryptophanate based on the total weight of the formulation. In certain embodiments, the formulation further comprises about 0.30% to 1.00% sodium caprylate based on the total weight of the formulation. In certain embodiments, the formulation further comprises about 0.20% to 0.60% formic acid based on the total weight of the formulation. In certain embodiments, the formulation further comprises about 0.15% to 0.60% acetic acid based on the total weight of the formulation.

In one embodiment, the formulation provided herein comprises about 0.08% to 0.12% Compound 1, about 40.00% to 55.00% human albumin, about 10.00% to 55.00% sucrose, about 3.00% to 4.50% citric acid based on the total weight of the formulation. In certain embodiments, the formulation further comprises about 1.50% to 2.50% sodium chloride based on the total weight of the formulation. In certain embodiments, the formulation further comprises about 0.80% to 1.50% sodium N-acetyltryptophanate based on the total weight of the formulation. In certain embodiments, the formulation further comprises about 0.50% to 1.00% sodium caprylate based on the total weight of the formulation. In certain embodiments, the formulation further comprises about 0.30% to 0.50% formic acid based on the total weight of the formulation. In certain embodiments, the formulation further comprises about 0.20% to 0.60% acetic acid based on the total weight of the formulation.

In one embodiment, the formulation provided herein comprises about 0.08% to 0.12% Compound 1, about 40.00% to 55.00% human albumin, about 10.00% to 55.00% sucrose, about 30.00% to 4.50% citric acid, about 1.50% to 2.50% sodium chloride, about 0.80% to 1.50% sodium N-acetyltryptophanate, about 0.50% to 1.00% sodium caprylate, about 0.30% to 0.50% formic acid and about 0.20% to 0.60% acetic acid based on the total weight of the formulation.

In one embodiment, the formulation provided herein comprises about 0.08% to 0.12% Compound 1, about 40.00% to 55.00% human albumin, about 10.00% to 25.00% trehalose, about 15% to 30% mannitol, about 30.00% to 4.50% citric acid, about 1.50% to 2.50% sodium chloride, about 0.80% to 1.50% sodium N-acetyltryptophanate, about 0.50% to 1.00% sodium caprylate, about 0.30% to 0.50% formic acid and about 0.20% to 0.60% acetic acid based on the total weight of the formulation.

In one embodiment, the formulation provided herein comprises about 0.03% to 0.06% Compound 1 based on the total weight of the formulation. In one embodiment, the formulation provided herein comprises about 0.030%, 0.035%, 0.040%, 0.042%, 0.045%, 0.050%, 0.051%, 0.055% or 0.060% Compound 1 based on the total weight of the formulation. In one embodiment, the formulation provided herein comprises about 0.042% Compound 1 based on the total weight of the formulation.

In one embodiment, the formulation provided herein comprises about 0.080%, 0.10% or 0.11% Compound 1 based on the total weight of the formulation.

In another aspect, provided herein is a lyophilized formulation that comprises Compound 1 in an amount of about 0.5 mg to about 3.5 mg in a 50 cc vial. In one aspect, Compound 1 is present in an amount of about 0.6, 0.9, 1.0, 1.2, 2.4 or 3 mg in a 50 cc vial. In one aspect, Compound 1 is present in an amount of about 0.6, 0.9, 1.0, 1.2, 2.4, 2.5 or 3 mg in a 50 cc vial. In one aspect, Compound 1 is present in an amount of about 1 mg in a 50 cc vial.

In another aspect, provided herein is a lyophilized formulation that comprises Compound 1 in an amount of about 5 mg in a 100 cc vial. In another aspect, provided herein is a lyophilized formulation that comprises Compound 1 in an amount of about 0.5 mg in a 10 cc vial.

In one embodiment, the formulation provided herein comprises about 35.00% to 50.00% human albumin based on the total weight of the formulation. In one embodiment, the formulation provided herein comprises about 35.00%, 37.00%, 39.00%, 41.00%, 42.29%, 45.00%, 47.00% or 50.00% human albumin based on the total weight of the formulation. In one embodiment, the formulation provided herein comprises about 42% human albumin based on the total weight of the formulation. In one embodiment, the formulation provided herein comprises about 42.29% human albumin based on the total weight of the formulation. In embodiment, the human albumin is recombinant human albumin.

In one embodiment, the formulation provided herein comprises about 40.00% to 55.00% human albumin based on the total weight of the formulation. In one embodiment, the formulation provided herein comprises about 40.00%, 40.03%, 40.13%, 50.00%, 50.79% or 53.51% human albumin based on the total weight of the formulation. In one embodiment, the formulation provided herein comprises about 40.13% human albumin based on the total weight of the formulation.

In another aspect, provided herein is a lyophilized formulation that comprises human albumin in an amount of about 500 mg to about 2500 mg in a 50 cc vial. In one aspect, human albumin is in an amount of about 600 mg to about 1200 mg in a 50 cc vial. In one aspect, human albumin is in an amount of about 600 mg, about 1000 mg, about 1200 mg or about 2500 mg in a 50 cc vial. In one aspect, human albumin is in an amount of about 600 mg or about 1000 mg in a 50 cc vial. In one aspect, human albumin is in an amount of about 1000 mg in a 50 cc vial. In embodiment, the human albumin is recombinant human albumin.

In another aspect, provided herein is a lyophilized formulation that comprises human albumin in an amount of about 1250 mg in a 50 cc vial. In another aspect, provided herein is a lyophilized formulation that comprises human albumin in an amount of about 2500 mg in a 100 cc vial. In another aspect, provided herein is a lyophilized formulation that comprises human albumin in an amount of about 250 mg in a 10 cc vial.

In one embodiment, the formulation provided herein comprises about 40.00% to 60.00% sucrose based on the total weight of the formulation. In one embodiment, the formulation provided herein comprises about 40.00%, 42.00%, 45.00%, 47.00%, 49.00%, 50.75%, 51.00%, 52.00%, 55.00%, 57.00% or 60%% sucrose based on the total weight of the formulation. In one embodiment, the formulation provided herein comprises about 51% sucrose based on the total weight of the formulation. In one embodiment, the formulation provided herein comprises about 50.75% sucrose based on the total weight of the formulation.

In one embodiment, the formulation provided herein comprises about 10.00% to 55.00% sucrose based on the total weight of the formulation. In one embodiment, the formulation provided herein comprises about 10.70%, 20.32%, 52.84% or 52.97% sucrose based on the total weight of the formulation. In one embodiment, the formulation provided herein comprises about 52.97% sucrose based on the total weight of the formulation.

In one embodiment, the formulation provided herein comprises about 15.00% to 30.00% mannitol based on the total weight of the formulation. In one embodiment, the formulation provided herein comprises about 20.00% to 27.00% mannitol based on the total weight of the formulation.

In one embodiment, the formulation provided herein comprises about 10.00% to 25.00% sucrose based on the total weight of the formulation. In one embodiment, the formulation provided herein comprises about 10.70% or 20.32% sucrose based on the total weight of the formulation. In one embodiment, the formulation provided herein comprises about 10.00% to 25.00% sucrose and about 15.00% to 30.00% mannitol based on the total weight of the formulation. In one embodiment, the formulation provided herein comprises about 20.32% sucrose and about 20.32% mannitol based on the total weight of the formulation. In one embodiment, the formulation provided herein comprises about 10.70% sucrose and about 26.76% mannitol based on the total weight of the formulation.

In one embodiment, the formulation provided herein comprises about 10.00% to 25.00% trehalose based on the total weight of the formulation. In one embodiment, the formulation provided herein comprises about 10.70% or 20.32% trehalose based on the total weight of the formulation. In one embodiment, the formulation provided herein comprises about 10.00% to 25.00% trehalose and about 15.00% to 30.00% mannitol based on the total weight of the formulation. In one embodiment, the formulation provided herein comprises about 20.32% trehalose and about 20.32% mannitol based on the total weight of the formulation. In one embodiment, the formulation provided herein comprises about 10.70% trehalose and about 26.76% mannitol based on the total weight of the formulation.

In another aspect, provided herein is a lyophilized formulation that comprises sucrose in an amount of about 400 mg to about 3000 mg in a 50 cc vial. In one aspect, sucrose is in an amount of about 1000 mg to about 2000 mg in a 50 cc vial. In one aspect, sucrose is in an amount of about 1200 mg, about 1608 mg, about 1644 mg, about 1920 mg, or about 3000 mg in a 50 cc vial. In one aspect, sucrose is in an amount of about 1200 mg in a 50 cc vial.

In another aspect, provided herein is a lyophilized formulation that comprises sucrose in an amount of about 1650 mg in a 50 cc vial. In one aspect, sucrose is in an amount of about 3300 mg in a 100 cc vial.

In another aspect, provided herein is a lyophilized formulation that comprises sucrose in an amount of about 100 mg in a 10 cc vial. In one aspect, sucrose is in an amount of about 50 mg in a 10 cc vial. In another aspect, provided herein is a lyophilized formulation that comprises sucrose in an amount of about 100 mg and mannitol in an amount of 100 mg in a 10 cc vial. In another aspect, provided herein is a lyophilized formulation that comprises sucrose in an amount of about 50 mg and mannitol in an amount of 125 mg in a 10 cc vial.

In another aspect, provided herein is a lyophilized formulation that comprises trehalose in an amount of about 100 mg in a 10 cc vial. In another aspect, provided herein is a lyophilized formulation that comprises trehalose in an amount of about 50 mg in a 10 cc vial. In another aspect, provided herein is a lyophilized formulation that comprises trehalose in an amount of about 100 mg and mannitol in an amount of 100 mg in a 10 cc vial. In another aspect, provided herein is a lyophilized formulation that comprises trehalose in an amount of about 50 mg and mannitol in an amount of 125 mg in a 10 cc vial.

In one embodiment, the formulation provided herein comprises about 2.5% to 4.5% citric acid based on the total weight of the formulation. In one embodiment, the formulation provided herein comprises about 2.5%, 2.8%, 3.0%, 3.2%, 3.3%, 3.5%, 3.6%, 4.0%, 4.3% or 4.5% citric acid based on the total weight of the formulation. In one embodiment, the formulation provided herein comprises about 3.08%, 3.07%, 3.9% or 4.1% citric acid based on the total weight of the formulation. In one embodiment, the formulation provided herein comprises about 3.7% citric acid based on the total weight of the formulation. In one embodiment, the formulation provided herein comprises about 3.66% citric acid based on the total weight of the formulation. In one embodiment, the formulation provided herein comprises about 3.08% citric acid based on the total weight of the formulation.

In another aspect, provided herein is a lyophilized formulation that comprises citric acid in an amount of about 20 mg to about 200 mg in a 50 cc vial. In one aspect, citric acid is in an amount of about 50 mg to about 100 mg in a 50 cc vial. In one aspect, citric acid is in an amount of about 23.1 mg, about 46.1 mg, about 86.5 mg, about 103.7 mg, or about 192.1 mg in a 50 cc vial. In one aspect, citric acid is in an amount of about 23.1 mg, about 46.1 mg, about 86.5 mg, about 96.1 mg, about 103.7 mg, or about 192.1 mg in a 50 cc vial.

In one aspect, citric acid is in an amount of about 86.5 mg in a 50 cc vial. In one aspect, citric acid is in an amount of about 192.1 mg in a 100 cc vial. In one aspect, citric acid is in an amount of about 19.2 mg in a 10 cc vial.

In certain embodiments, the formulation comprises about 1.0% to 3.0% sodium chloride based on the total weight of the formulation. In certain embodiments, the formulation comprises about 1.0%, 1.2%, 1.4%, 1.6%, 1.7%, 1.8%, 2.0%, 2.3%, 2.5%, 2.7% or 3.0% sodium chloride based on the total weight of the formulation. In certain embodiments, the formulation comprises about 1.0% to 3.0% sodium chloride based on the total weight of the formulation. In certain embodiments, the formulation comprises about 1.7%, 2.1%, 2.2% or 2.3% sodium chloride based on the total weight of the formulation. In certain embodiments, the formulation comprises about 1.8% sodium chloride based on the total weight of the formulation.

In certain embodiments, the formulation comprises about 1.79% sodium chloride based on the total weight of the formulation. In certain embodiments, the formulation comprises about 1.7% sodium chloride based on the total weight of the formulation.

In another aspect, provided herein is a lyophilized formulation that comprises sodium chloride in an amount of about 20 mg to about 125 mg in a 50 cc vial. In one aspect, sodium chloride is in an amount of about 40 mg to about 60 mg in a 50 cc vial. In one aspect, sodium chloride is in an amount of about 25.1 mg, about 42.4 mg, or about 50.8 mg in a 50 cc vial. In one aspect, sodium chloride is in an amount of about 42.4 mg in a 50 cc vial.

In one aspect, sodium chloride is in an amount of about 53 mg in a 50 cc vial. In one aspect, sodium chloride is in an amount of about 105.9 mg in a 100 cc vial. In one aspect, sodium chloride is in an amount of about 5.4 mg in a 10 cc vial.

In certain embodiments, the formulation comprises about 0.50% to 1.50% sodium N-acetyltryptophanate based on the total weight of the formulation. In certain embodiments, the formulation comprises about 0.5%, 0.7%, 0.9%, 1.0%, 1.3%, 1.1% or 1.5% sodium N-acetyltryptophanate based on the total weight of the formulation. In certain embodiments, the formulation comprises about 0.5%, 0.7%, 0.9%, 1.0%, 1.3%, or 1.5% sodium N-acetyltryptophanate based on the total weight of the formulation. In certain embodiments, the formulation comprises about 0.9% sodium N-acetyltryptophanate based on the total weight of the formulation. In certain embodiments, the formulation comprises about 0.91% sodium N-acetyltryptophanate based on the total weight of the formulation.

In certain embodiments, the formulation comprises about 1.1% sodium N-acetyltryptophanate based on the total weight of the formulation.

In another aspect, provided herein is a lyophilized formulation that comprises sodium N-acetyltryptophanate in an amount of about 10 mg to about 35 mg in a 50 cc vial. In one aspect, sodium N-acetyltryptophanate is in an amount of about 10 mg to about 30 mg in a 50 cc vial. In one aspect, sodium N-acetyltryptophanate is in an amount of about 12.9 mg, about 21.5 mg, or about 25.8 mg in a 50 cc vial. In one aspect, sodium N-acetyltryptophanate is in an amount of about 25.8 mg in a 50 cc vial. In one aspect, sodium N-acetyltryptophanate is in an amount of about 26.8 mg in a 50 cc vial.

In one aspect, sodium N-acetyltryptophanate is in an amount of about 53.6 mg in a 100 cc vial. In one aspect, sodium N-acetyltryptophanate is in an amount of about 10.6 mg in a 10 cc vial.

In certain embodiments, the formulation comprises about 0.30% to 0.70% sodium caprylate based on the total weight of the formulation. In certain embodiments, the formulation comprises about 0.3%, 0.4%, 0.5%, 0.6% or 0.7% sodium caprylate based on the total weight of the formulation. In certain embodiments, the formulation comprises about 0.6% sodium caprylate based on the total weight of the formulation. In certain embodiments, the formulation comprises about 0.56% sodium caprylate based on the total weight of the formulation.

In certain embodiments, the formulation comprises about 0.53% sodium caprylate based on the total weight of the formulation. In certain embodiments, the formulation comprises about 0.68% sodium caprylate based on the total weight of the formulation. In certain embodiments, the formulation comprises about 0.71% sodium caprylate based on the total weight of the formulation.

In another aspect, provided herein is a lyophilized formulation that comprises sodium caprylate in an amount of about 3 mg to about 35 mg in a 50 cc vial. In one aspect, sodium caprylate is in an amount of about 4 mg to about 34 mg in a 50 cc vial. In one aspect, sodium caprylate is in an amount of about 4.0 mg, about 8.0 mg, about 13.3 mg, about 16.0 mg, or about 33.2 mg in a 50 cc vial. In one aspect, sodium caprylate is in an amount of about 13.3 mg in a 50 cc vial.

In one embodiment, the formulation provided herein comprises about 0.04% Compound 1, about 40.29% human albumin, about 50.75% sucrose, and about 30.65% citric acid based on the total weight of the formulation. In certain embodiments, the formulation further comprises about 1.79% sodium chloride based on the total weight of the formulation. In certain embodiments, the formulation further comprises about 0.91% sodium N-acetyltryptophanate based on the total weight of the formulation. In certain embodiments, the formulation further comprises about 0.56% sodium caprylate based on the total weight of the formulation.

In one embodiment, the formulation provided herein comprises about 0.04% Compound 1, about 42.29% human albumin, about 50.75% sucrose, about 30.65% citric acid, about 1.79% sodium chloride, about 0.91% sodium N-acetyltryptophanate and about 0.56% sodium caprylate based on the total weight of the formulation.

In one embodiment, provided herein is a lyophilized formulation provided herein comprises about 0.04% Compound 1, about 42.29% human albumin, about 50.75% sucrose, about 30.65% citric acid, about 1.80% sodium chloride, about 0.91% sodium N-acetyltryptophanate and about 0.56% sodium caprylate based on the total weight of the lyophilized formulation.

In one embodiment, the formulation provided herein comprises about 0.08% Compound 1, about 4.13% human albumin, about 52.97% sucrose, about 3.08% citric acid, about 1.7% sodium chloride, about 0.86% sodium N-acetyltryptophanate, about 0.53% sodium caprylate, about 0.36% formic acid and about 0.28% acetic acid based on the total weight of the formulation.

In one embodiment, the formulation provided herein comprises about 0.10% Compound 1, about 50.79% human albumin, about 2.32% sucrose, about 2.32% mannitol, about 3.90% citric acid, about 2.15% sodium chloride, about 1.09% sodium N-acetyltryptophanate, about 0.68% sodium caprylate, about 0.46% formic acid and about 0.20% acetic acid based on the total weight of the formulation.

In one embodiment, the formulation provided herein comprises about 0.11% Compound 1, about 53.51% human albumin, about 10.70% sucrose, about 26.75% mannitol, about 4.11% citric acid, about 2.27% sodium chloride, about 1.15% sodium N-acetyltryptophanate, about 0.71% sodium caprylate, about 0.48% formic acid and about 0.21% acetic acid based on the total weight of the formulation.

In one embodiment, the formulation provided herein comprises about 0.10% Compound 1, about 50.79% human albumin, about 2.32% trehalose, about 2.32% mannitol, about 3.90% citric acid, about 2.15% sodium chloride, about 1.09% sodium N-acetyltryptophanate, about 0.68% sodium caprylate, about 0.46% formic acid and about 0.20% acetic acid based on the total weight of the formulation.

In one embodiment, the formulation provided herein comprises about 0.11% Compound 1, about 53.51% human albumin, about 10.70% trehalose, about 26.75% mannitol, about 4.11% citric acid, about 2.27% sodium chloride, about 1.15% sodium N-acetyltryptophanate, about 0.71% sodium caprylate, about 0.48% formic acid and about 0.21% acetic acid based on the total weight of the formulation.

In another aspect is a lyophilized formulation that comprises Compound 1 in an amount of about 0.5 mg to about 3.5 mg, human albumin in an amount of about 500 mg to about 2500 mg, sucrose in an amount of about 400 mg to about 3000 mg, and citric acid in an amount of about 20 mg to about 200 mg in a 50 cc vial. In one aspect, the lyophilized formulation further comprises sodium chloride in an amount of about 20 mg to about 125 mg in a 50 cc vial. In one aspect, the lyophilized formulation further comprises sodium N-acetyltryptophanate in an amount of about 10 mg to about 35 mg in a 50 cc vial. In one aspect, the lyophilized formulation further comprises sodium caprylate in an amount of about 3 mg to about 35 mg in a 50 cc vial.

In another aspect is a lyophilized formulation that comprises Compound 1 in an amount of about 0.5 mg to about 1.5 mg, human albumin in an amount of about 600 mg to about 1200 mg, sucrose in an amount of about 1000 mg to about 1200 mg, and citric acid in an amount of about 50 mg to about 100 mg in a 50 cc vial. In one aspect, the lyophilized formulation further comprises sodium chloride in an amount of about 20 mg to about 125 mg in a 50 cc vial. In one aspect, the lyophilized formulation further comprises sodium N-acetyltryptophanate in an amount of about 10 mg to about 30 mg in a 50 cc vial. In one aspect, the lyophilized formulation further comprises sodium caprylate in an amount of about 4 mg to about 34 mg in a 50 cc vial.

In another aspect is a lyophilized formulation that comprises Compound 1 in an amount of about 1 mg, human albumin in an amount of about 1000 mg, sucrose in an amount of about 1200 mg and citric acid in an amount of about 86.5 mg in a 50 cc vial. In one aspect, the lyophilized formulation further comprises sodium chloride in an amount of about 42.4 mg in a 50 cc vial. In one aspect, the lyophilized formulation further comprises sodium N-acetyltryptophanate in an amount of about 25.8 mg in a 50 cc vial. In one aspect, the lyophilized formulation further comprises sodium caprylate in an amount of about 13.3 mg in a 50 cc vial.

In another aspect is a lyophilized formulation that comprises Compound 1 in an amount of about 5 mg, human albumin in an amount of about 2500 mg, sucrose in an amount of about 3300 mg and citric acid in an amount of about 192.1 mg in a 50 cc vial. In one aspect, the lyophilized formulation further comprises sodium chloride in an amount of about 105.9 mg in a 50 cc vial. In one aspect, the lyophilized formulation further comprises sodium N-acetyltryptophanate in an amount of about 53.6 mg in a 50 cc vial. In one aspect, the lyophilized formulation further comprises sodium caprylate in an amount of about 33.2 mg in a 50 cc vial. In one aspect, the lyophilized formulation further comprises about 22.50 mg formic acid and about 17.50 mg acetic acid in a 50 cc vial.

In another aspect is a lyophilized formulation that comprises Compound 1 in an amount of about 6 mg, human albumin in an amount of about 3000 mg, trehalose in an amount of about 1200 mg, mannitol in an amount of about 1200 mg, and citric acid in an amount of about 230 mg in a 100 cc vial. In one aspect, the lyophilized formulation further comprises sodium chloride in an amount of about 127 mg in a 100 cc vial. In one aspect, the lyophilized formulation further comprises sodium N-acetyltryptophanate in an amount of about 64 mg in a 100 cc vial. In one aspect, the lyophilized formulation further comprises sodium caprylate in an amount of about 40 mg in a 100 cc vial. In one aspect, the lyophilized formulation further comprises about 27 mg formic acid and about 12 mg acetic acid based in a 100 cc vial.

In one aspect provided herein is a formulation in a 50 cc vial, that consists essentially of Compound 1 at an amount that provides about 1 mg to about 1.1 mg 2-(4-chlorophenyl)-N-((2-(2,6-dioxopiperidin-3-yl)-1-oxoisoindolin-5-yl)methyl)-2,2-difluoroacetamide, about 1000 mg human albumin, about 1200 mg sucrose and about 86.5 mg citric acid.

In one embodiment, provided herein is an aqueous formulation comprising Compound 1 in an amount of about 50 μg/mL, human albumin in an amount of about 50 mg/mL, sucrose in an amount of about 60 mg/mL, and citric acid in an amount of about 22.5 mM. In one aspect, the aqueous formulation further comprises formic acid in an amount of about 0.41 μg/mL. In one aspect, the aqueous formulation further comprises sodium N-acetyltryptophanate in an amount of about 4 mM. In one aspect, the aqueous formulation further comprises sodium caprylate in an amount of about 4 mM.

In certain embodiments, the formulations provided herein are lyophilized formulations. In certain embodiments, the formulations provided herein are aqueous formulations. In certain embodiments, the formulations provided herein are reconstituted formulations obtained in a pharmaceutically acceptable solvent to produce a pharmaceutically acceptable solution.

In certain embodiments, the formulation upon reconstitution has a pH of about 4 to 5. In one embodiment, the formulation upon reconstitution has a pH of about 4, 4.1, 4.2, 4.3, 4.4, 4.5, 4.6, 4.7, 4.8, 4.9 or 5.

In certain embodiments, provided herein is a container comprising a formulation provided herein. In certain embodiments, provided herein is a container comprising a lyophilized formulation provided herein. In one aspect, the container is a glass vial. In one aspect, the container is a 20 cc glass vial.

The lyophilized formulations of Compound 1 provided herein can be administered to a patient in need thereof using standard therapeutic methods for delivering Compound 1 including, but not limited to, the methods described herein. In one embodiment, the lyophilized formulations provided herein are reconstituted in a pharmaceutically acceptable solvent to produce a pharmaceutically acceptable solution, wherein the solution is administered (such as by intravenous injection) to the patient.

The lyophilized formulation provided herein can be reconstituted for parenteral administration to a patient using any pharmaceutically acceptable diluent. Such diluents include, but are not limited to water for injection.

Any quantity of diluent may be used to constitute the lyophilized formulation such that a suitable solution for injection is prepared. Accordingly, the quantity of the diluent must be sufficient to dissolve the lyophilized formulation. In one embodiment, 4-6 mL of a diluent are used to constitute the lyophilized formulation to yield a final concentration of, about 0.1-0.3 mg/mL, about 0.15 mg/mL, or about 0.2 mg/mL of Compound 1. In certain embodiments, the final concentration of Compound 1 in the reconstituted solution is about 0.2 mg/mL. In certain embodiment, depending on the required dose, multiple vials may be used for reconstitution.

The reconstituted solutions of lyophilized formulation can be stored and used within up to about 24 hours, about 12 hours or about 8 hours. In some embodiments, the solution is used within 8 hour of preparation. In some embodiments, the solution is used within 5 hour of preparation. In some embodiments, the solution is used within 1 hour of preparation.

Process for Preparing Formulations

The formulations comprising human albumin can be prepared by any of the methods known in the art and as described herein, but all methods include the step of bringing the active ingredient into association with the pharmaceutically acceptable excipient, which constitutes one or more necessary ingredients (such as bulking agent and/or buffer).

In one aspect, the formulations provided herein are prepared by adding a mixture of sucrose and 20% human albumin to a citrate buffer in water to obtain a sucrose/human albumin solution, and adding a solution of Compound 1 in formic acid to the sucrose/human albumin solution to obtain a drug solution. In one aspect, the drug solution is filtered to obtain a filtered solution, and the filtered solution is lyophilized to obtain a lyophilized formulation.

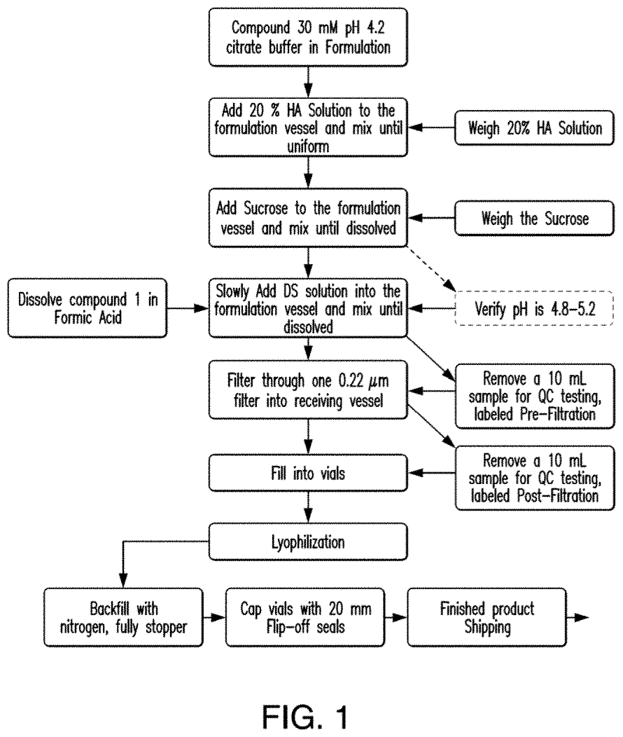

In one aspect, the methods for preparing the formulations provided herein comprise the one or more of the following steps: (i) adding a mixture of sucrose and 20% human albumin to citrate buffer in water to obtain a sucrose/human albumin solution, (ii) mixing a solution of Compound 1 in formic acid to the sucrose/human albumin solution to obtain a suspension, (iii) filtering the suspension to obtain a filtered solution, and (iv) lyophilizing the filtered solution in a vial. Flow charts illustrating exemplary processes are provided in FIGS. 1, 20 and 22.

In one aspect, the formulations provided herein are prepared by adding a mixture of trehalose, mannitol and 20% human albumin to a citrate buffer in water to obtain a trehalose/mannitol/human albumin solution, adding a solution of Compound 1 in formic acid to the trehalose/mannitol/human albumin solution to obtain a mixture, and adding acetic acid to the mixture to obtain a drug solution. In one aspect, the drug solution is filtered to obtain a filtered solution, and the filtered solution is lyophilized to obtain a lyophilized formulation.

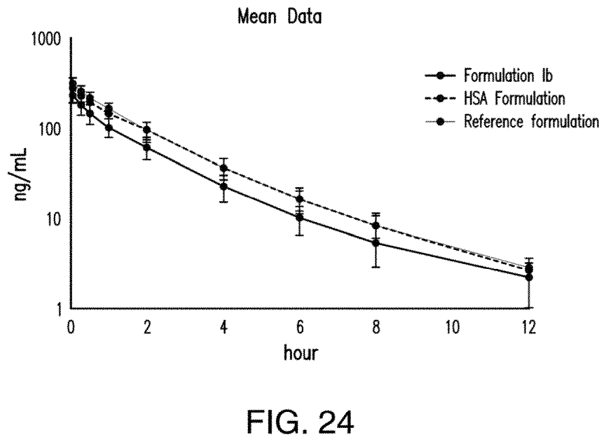

In one aspect, the methods for preparing the formulations provided herein comprise the one or more of the following steps: (i) adding a mixture of trehalose, mannitol and 20% human albumin to a citrate buffer in water to obtain a trehalose/mannitol/human albumin solution, (ii) adding a solution of Compound 1 in formic acid to the trehalose/mannitol/human albumin solution to obtain a mixture, (iii) adding acetic acid to the mixture to obtain a drug solution, and (iv) lyophilizing the filtered solution in a vial. A flow chart illustrating an exemplary process is provided in FIG. 24.

In one embodiment, the vial is sealed under nitrogen after lyophilization.

In one aspect, the lyophilization process contains three stages: freezing, primary drying, and secondary drying. A liquid formulation is transformed to a lyophilized powder form by going through complete solidification through freezing stage, sublimation of ice and solvents through primary drying, and desorption of residual moisture and solvents through secondary drying. The shelf temperature and chamber pressure in the primary drying and secondary drying are controlled to obtain the desired quality of the finished drug product. In one aspect of the process, the cake appearance and structure is characterized by visual inspection.

Kits

Pharmaceutical packs or kits which comprise pharmaceutical compositions or dosage forms provided herein are also provided. Exemplary kits include notice in the form prescribed by a governmental agency regulating the manufacture, use or sale of pharmaceuticals products, which notice reflects approval by the agency of manufacture, use or sale for human administration.

Methods of Use and Formulations of Compound 1 for Use in Such Methods

In one embodiment, provided herein is a method of treating and preventing cancer, which comprises administering to a patient a formulation of Compound 1 provided herein. Provided herein is a formulation of Compound 1 for use in such a method of treating and preventing cancer.

In another embodiment, provided herein is a method of managing cancer, which comprises administering to a patient a formulation of Compound 1 provided herein. Provided herein is Compound 1 for use in such a method of managing cancer.

In one embodiment, the methods provided herein comprise administering a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one embodiment, the methods provided herein comprise administering a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

Also provided herein are methods of treating patients who have been previously treated for cancer but are non-responsive to cancer therapies, as well as those who have not previously been treated. Also encompassed are methods of treating patients regardless of patient's age, although some diseases or disorders are more common in certain age groups. Further encompassed are methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

In one embodiment, provided herein are methods for improving the Eastern Cooperative Oncology Group Performance Status (ECOG) of a cancer patient, comprising administering an effective amount of a formulation of Compound 1 to the patient. Provided herein is a formulation of Compound 1 for use in improving the Eastern Cooperative Oncology Group Performance Status (ECOG) of a cancer patient.

In one embodiment, provided herein are methods for improving the Eastern Cooperative Oncology Group Performance Status (ECOG) of a cancer patient, comprising administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in methods for improving the Eastern Cooperative Oncology Group Performance Status (ECOG) of a cancer patient, comprising administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment, provided herein are methods for improving the Eastern Cooperative Oncology Group Performance Status (ECOG) of a cancer patient, comprising administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in methods for improving the Eastern Cooperative Oncology Group Performance Status (ECOG) of a cancer patient, comprising administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment, provided herein are methods for inhibition of disease progression, inhibition of tumor growth, reduction of primary tumor, relief of tumor-related symptoms, inhibition of tumor secreted factors, delaying appearance of primary or secondary tumors, slowing development of primary or secondary tumors, decreasing occurrence of primary or secondary tumors, slowing or decreasing severity of secondary effects of disease, arresting tumor growth and regression of tumors, increasing time to progression, increasing progression free survival, increasing overall survival in a cancer patient, or one or more thereof, comprising administering an effective amount of a formulation of Compound 1 to the patient. Provided herein is Compound 1 for use in all such methods in a cancer patient, or one or more thereof, comprising administering an effective amount of a formulation of Compound 1 to the patient.

In one embodiment, provided herein are methods for inhibition of disease progression, inhibition of tumor growth, reduction of primary tumor, relief of tumor-related symptoms, inhibition of tumor secreted factors, delaying appearance of primary or secondary tumors, slowing development of primary or secondary tumors, decreasing occurrence of primary or secondary tumors, slowing or decreasing severity of secondary effects of disease, arresting tumor growth and regression of tumors, increasing time to progression, increasing progression free survival, increasing overall survival in a cancer patient, or one or more thereof, comprising administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is Compound 1 for use in all such methods in a cancer patient, or one or more thereof, comprising administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment, provided herein are methods for inhibition of disease progression, inhibition of tumor growth, reduction of primary tumor, relief of tumor-related symptoms, inhibition of tumor secreted factors, delaying appearance of primary or secondary tumors, slowing development of primary or secondary tumors, decreasing occurrence of primary or secondary tumors, slowing or decreasing severity of secondary effects of disease, arresting tumor growth and regression of tumors, increasing time to progression, increasing progression free survival, increasing overall survival in a cancer patient, or one or more thereof, comprising administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is Compound 1 for use in all such methods in a cancer patient, or one or more thereof, comprising administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In certain embodiments, the cancer is a solid tumor or a hematological cancer. In certain embodiments, the cancer is interleukin-3 (IL-3) independent. In certain embodiments, the cancer is a solid tumor. In certain embodiments, the solid tumor is metastatic. In certain embodiments, the solid tumor is drug-resistant.

In certain embodiments, cancer refers to a disease of skin tissues, organs, blood, and vessels. In certain embodiments, the cancer is a solid tumor, including, but not limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, colorectal cancer, including stage 3 and stage 4, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, carcinoma, including papillary thyroid carcinoma, follicular thyroid carcinoma, and medullary thyroid carcinoma, and leiomyoma.

In certain embodiments, the cancer is a solid tumor, including, but not limited to, cancers of the skin, central nervous system, soft tissue, salivary gland, ovary, kidney, lung, bone, stomach, endometrium, pancreas, urinary tract, thyroid, upper aerodigestive tract, breast, large intestine, oesophagus, prostate, liver, autonomic ganglia, and malignant pleural mesothelioma.

In certain embodiments, the solid tumor is hepatocellular carcinoma, prostate cancer, ovarian cancer, or glioblastoma.

In certain embodiments, the solid tumor is breast cancer, kidney cancer, pancreatic cancer, gastrointestinal cancer, lung cancer, neuroendocrine tumor (NET), or renal cell carcinoma (RCC).

In certain embodiments, the cancer is a hematological cancer. In certain embodiments, the hematological cancer is metastatic. In certain embodiments, the hematological cancer is drug resistant to at least one anti-cancer therapy. In certain embodiments the hematological cancer is relapsed or refractory to at least one anti-cancer therapy.

In one embodiment, the hematological cancer is multiple myeloma (MM). In one embodiment, the hematological cancer is relapsed/refractory (R/R) MM. In one embodiment, the patient having R/R MM has impaired renal function.

In one embodiment provided herein is a method for achieving a stringent complete remission (sCR) in anMM patient, wherein the method comprises administering an effective amount of a formulation of Compound 1 to the patient. Provided herein is a formulation of Compound 1 for use in a method for achieving a stringent complete remission (sCR) in anMM patient, wherein the method comprises administering an effective amount of a formulation of Compound 1 to the patient.

In one embodiment provided herein is a method for achieving a stringent complete remission (sCR) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a stringent complete remission (sCR) in an MM patient, wherein the method comprises administering an effective amount of a formulation of Compound 1 in combination with one or more second agents selected from glucocorticoid receptor agonists, IL-113 receptor antagonists, interleukin-1β blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a stringent complete remission (sCR) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a stringent complete remission (sCR) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a complete remission (CR) in an MM patient, wherein the method comprises administering an effective amount of a formulation of Compound 1 to the patient. Provided herein is a formulation of Compound 1 for use in a method for achieving a complete remission (CR) in an MM patient, wherein the method comprises administering an effective amount of a formulation of Compound 1 to the patient.

In one embodiment provided herein is a method for achieving a complete remission (CR) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a complete remission (CR) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a very good partial response (VGPR) in an MM patient, wherein the method comprises administering an effective amount of a formulation of Compound 1 to the patient. Provided herein is a formulation of Compound 1 for use in a method for achieving a very good partial response (VGPR) in an MM patient.

In one embodiment provided herein is a method for achieving a very good partial response (VGPR) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a very good partial response (VGPR) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a very good partial response (VGPR) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a very good partial response (VGPR) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial response (PR) in an MM patient, wherein the method comprises administering an effective amount of a formulation of Compound 1 to the patient. Provided herein is a formulation of Compound 1 for use in a method for achieving a partial response in an MM patient.

In one embodiment provided herein is a method for achieving a partial response (PR) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial response (PR) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial response (PR) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial response (PR) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a stable disease (SD) in an MM patient, wherein the method comprises administering an effective amount of a formulation of Compound 1 to the patient. Provided herein is a formulation of Compound 1 for use in a method for achieving a stable disease in an MM patient.

In one embodiment provided herein is a method for achieving a stable disease (SD) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a stable disease (SD) in an MM patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a stable disease (SD) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a stable disease (SD) in an MM patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment, the hematological cancer is acute myelogenous leukemia (AML). In one embodiment, the hematological cancer is acute lymphocytic leukemia (ALL). In one embodiment, the hematological cancer is adult T-cell leukemia. In one embodiment, the hematological cancer is chronic lymphocytic leukemia (CLL). In one embodiment, the hematological cancer is hairy cell leukemia. In one embodiment, the hematological cancer is myelodysplasia. In one embodiment, the hematological cancer is a myeloproliferative disorder or myeloproliferative neoplasm (MPN). In one embodiment, the hematological cancer is chronic myelogenous leukemia (CML). In one embodiment, the hematological cancer is myelodysplastic syndrome (MDS). In one embodiment, the hematological cancer is human lymphotropic virus-type 1 (HTLV-1) leukemia. In one embodiment, the hematological cancer is mastocytosis. In one embodiment, the hematological cancer is B-cell acute lymphoblastic leukemia. In one embodiment, the hematological cancer is CLL.

In one embodiment, provided herein are methods of treating, preventing, managing, and/or ameliorating a cancer selected from diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, mantle cell lymphoma (MCL), Hodgkin lymphoma (HL), non-Hodgkin lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, and ALK-positive large B-cell lymphoma in a subject, comprising the step of administering to the subject an amount of a formulation of Compound 1 provided herein effective to treat, prevent and/or manage the cancer. Thus, provided herein is a formulation of Compound 1 for use in all said methods of treating, preventing, managing, and/or ameliorating a cancer, wherein the cancer is selected from diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, mantle cell lymphoma (MCL), Hodgkin lymphoma (HL), non-Hodgkin lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, and ALK-positive large B-cell lymphoma in a subject. In some embodiments, the methods comprise the step of administering to the subject a formulation of Compound 1 provided herein in combination with a second active agent in amounts effective to treat, prevent and/or manage the cancer. In one embodiment, the hematological cancer is HL. In one embodiment, the hematological cancer is NHL. In one embodiment, the hematological cancer is indolent lymphoma including, for example, DLBCL, follicular lymphoma, and marginal zone lymphoma.

In one embodiment provided herein is a method for achieving a complete remission (CR) in an NHL patient, wherein the method comprises administering an effective amount of a formulation of Compound 1 to the patient. Provided herein is a formulation of Compound 1 for use in a method for achieving a complete remission (CR) in an NHL patient.

In one embodiment provided herein is a method for achieving a complete remission (CR) in an NHL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in an NHL patient, wherein the method comprises administering an effective amount of a formulation of Compound 1 in combination with one or more second agents selected from glucocorticoid receptor agonists, IL-113 receptor antagonists, interleukin-1β blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a complete remission (CR) in an NHL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in an NHL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial remission (PR) in an NHL patient, wherein the method comprises administering an effective amount of a formulation of Compound 1 to the patient. Provided herein is a formulation of Compound 1 for use in a method for achieving a partial remission (PR) in an NHL patient.

In one embodiment provided herein is a method for achieving a partial remission (PR) in an NHL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial remission (PR) in an NHL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial remission (PR) in an NHL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial remission (PR) in an NHL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a stable disease (SD) in an NHL patient, wherein the method comprises administering an effective amount of a formulation of Compound 1 to the patient. Provided herein is a formulation of Compound 1 for use in a method for achieving a stable disease (SD) in an NHL patient.

In one embodiment provided herein is a method for achieving a stable disease (SD) in an NHL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a stable disease (SD) in an NHL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a stable disease (SD) in an NHL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a stable disease (SD) in an NHL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment, provided herein are methods of treating, preventing, managing, and/or ameliorating leukemia by administering a therapeutically active amount of a formulation of Compound 1 to a subject. Thus, provided herein is a formulation of Compound 1 for use in such methods of treating, preventing, managing, and/or ameliorating leukemia.

In certain embodiments, the methods of treating, preventing and/or managing acute myeloid leukemia in a subject comprise the step of administering to the subject an amount of a formulation of Compound 1 provided herein effective to treat, prevent and/or manage acute myeloid leukemia. In some embodiments, the methods comprise the step of administering to the subject a formulation of Compound 1 provided herein in combination with a second active agent in amounts effective to treat, prevent and/or manage acute myeloid leukemia.

In one embodiment, the leukemia is acute myeloid leukemia (AML). In one embodiment, the AML is relapsed or refractory AML. In one embodiment, the AML is newly diagnosed AML. In another embodiment, the AML has FAB classification MO/1. In another embodiment, the AML has FAB classification M2. In another embodiment, the AML has FAB classification M3. In another embodiment, the AML has FAB classification M4. In another embodiment, the AML has FAB classification M5. In one embodiment, the AML is AML with at least one recurrent genetic abnormality (for example, AML with translocation between chromosomes 8 and 21; AML with translocation or inversion in chromosome 16; AML with translocation between chromosomes 9 and 11; APL (M3) with translocation between chromosomes 15 and 17; AML with translocation between chromosomes 6 and 9; AML with translocation or inversion in chromosome 3); AML (megakaryoblastic) with a translocation between chromosomes 1 and 22; AML with myelodysplasia-related changes; AML related to previous chemotherapy or radiation (for example, alkylating agent-related AML; or Topoisomerase II inhibitor-related AML); AML not otherwise categorized (for example, AML that does not fall into the above categories, i. e. AML minimally differentiated (MO); AML with minimal maturation (M1); AML with maturation (M2); Acute myelomonocytic leukemia (M4); Acute monocytic leukemia (M5); Acute erythroid leukemia (M6); Acute megakaryoblastic leukemia (M7); Acute basophilic leukemia; or Acute panmyelosis with fibrosis); Myeloid Sarcoma (also known as granulocytic sarcoma, chloroma or extramedullary myeloblastoma); or Undifferentiated and biphenotypic acute leukemias (also known as mixed phenotype acute leukemias). In one embodiment, the AML is characterized by a mutant allele of IDH2. In one aspect of this embodiment, the mutant allele of IDH2 has an R140X mutation. In another aspect of this embodiment, the R140X mutation is a R140Q mutation. In another aspect of this embodiment, the R140X mutation is a R140W mutation. In another aspect of this embodiment, the R140X mutation is a R140L mutation. In another aspect of this embodiment, the mutant allele of IDH2 has an R172X mutation. In another aspect of this embodiment, the R172X mutation is a R172K mutation. In another aspect of this embodiment, the R172X mutation is a R172G mutation.

In one embodiment, the AML is relapsed AML after allogeneic HSCT. In one embodiment, the AML is second or later relapsed AML. In one embodiment, the AML is refractory to initial induction or re-induction treatment. In certain embodiments, the AML is refractory to at least one induction/reinduction or consolidation therapy. In one embodiment, the AML is refractory to or relapsed after hypomethylating agent (HMA). As used herein, HMA failure is defined as primary progression or lack of clinical benefit after a minimum of 6 cycles or unable to tolerate HMA due to toxicity. In one embodiment, the AML is relapsed within 1 year of initial treatment (excluding AML with favorable-risk status).

In one embodiment provided herein is a method for achieving a morphologic leukemia free state in an AML patient, wherein the method comprises administering an effective amount of a formulation of Compound 1 to the patient. Provided herein is a formulation of Compound 1 for use in a method for achieving a morphologic leukemia free state in an AML patient.

In one embodiment provided herein is a method for achieving a morphologic leukemia free state in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a morphologic leukemia free state in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a morphologic leukemia free state in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a morphologic leukemia free state in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a morphologic complete remission in an AML patient, wherein the method comprises administering an effective amount of a formulation of Compound 1 to the patient. Provided herein is a formulation of Compound 1 for use in a method for achieving a morphologic complete remission in an AML patient.

In one embodiment provided herein is a method for achieving a morphologic complete remission in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a morphologic complete remission in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a morphologic complete remission in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a morphologic complete remission in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a cytogenetic complete remission (CRc) in an AML patient, wherein the method comprises administering an effective amount of a formulation of Compound 1 to the patient. Provided herein is a formulation of Compound 1 for use in a method for achieving a cytogenetic complete remission (CRc) in an AML patient, wherein the method comprises administering an effective amount of a formulation of Compound 1 to the patient.

In one embodiment provided herein is a method for achieving a cytogenetic complete remission (CRc) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a cytogenetic complete remission (CRc) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a cytogenetic complete remission (CRc) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a cytogenetic complete remission (CRc) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a molecular complete remission (CRm) in an AML patient, wherein the method comprises administering an effective amount of a formulation of Compound 1. Provided herein is a formulation of Compound 1 for use in a method for achieving a molecular complete remission (CRm) in an AML patient.

In one embodiment provided herein is a method for achieving a molecular complete remission (CRm) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a molecular complete remission (CRm) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a molecular complete remission (CRm) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a molecular complete remission (CRm) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a morphologic complete remission with incomplete blood recovery (CRi) in an AML patient, wherein the method comprises administering an effective amount of a formulation of Compound 1 to the patient. Provided herein is a formulation of Compound 1 for use in a method for achieving a morphologic complete remission with incomplete blood recovery (CRi) in an AML patient.

In one embodiment provided herein is a method for achieving a morphologic complete remission with incomplete blood recovery (CRi) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a morphologic complete remission with incomplete blood recovery (CRi) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a morphologic complete remission with incomplete blood recovery (CRi) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a morphologic complete remission with incomplete blood recovery (CRi) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial remission (PR) in an AML patient, wherein the method comprises administering an effective amount of a formulation of Compound 1 to the patient. Provided herein is a formulation of Compound 1 for use in a method for achieving a partial remission (PR) in an AML patient.

In one embodiment provided herein is a method for achieving a partial remission (PR) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial remission (PR) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial remission (PR) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial remission (PR) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a complete remission (CR) in an AML patient, wherein the method comprises administering an effective amount of a formulation of Compound 1 to the patient. Provided herein is a formulation of Compound 1 for use in a method for achieving a complete remission (CR) in an AML patient.

In one embodiment provided herein is a method for achieving a complete remission (CR) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in an AML patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a complete remission (CR) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in an AML patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In some embodiments, the methods provided herein encompass treating, preventing and/or managing acute lymphocytic leukemia (ALL) in a subject. The methods comprise the step of administering to the subject an amount of a formulation of Compound 1 provided herein effective to treat, prevent and/or manage ALL. In some embodiments, the methods comprise the step of administering to the subject a formulation of Compound 1 provided herein in combination with a second active agent in amounts effective to treat, prevent and/or manage ALL.

In some embodiments, ALL includes leukemia that originates in the blast cells of the bone marrow (B-cells), thymus (T-cells), and lymph nodes. The ALL can be categorized according to the French-American-British (FAB) Morphological Classification Scheme as L1—Mature-appearing lymphoblasts (T-cells or pre-B-cells), L2—Immature and pleomorphic (variously shaped) lymphoblasts (T-cells or pre-B-cells), and L3—Lymphoblasts (B-cells; Burkitt's cells). In one embodiment, the ALL originates in the blast cells of the bone marrow (B-cells). In one embodiment, the ALL originates in the thymus (T-cells). In one embodiment, the ALL originates in the lymph nodes. In one embodiment, the ALL is L1 type characterized by mature-appearing lymphoblasts (T-cells or pre-B-cells). In one embodiment, the ALL is L2 type characterized by immature and pleomorphic (variously shaped) lymphoblasts (T-cells or pre-B-cells). In one embodiment, the ALL is L3 type characterized by lymphoblasts (B-cells; Burkitt's cells). In certain embodiments, the ALL is T-cell leukemia. In one embodiment, the T-cell leukemia is peripheral T-cell leukemia. In another embodiment, the T-cell leukemia is T-cell lymphoblastic leukemia. In another embodiment, the T-cell leukemia is cutaneous T-cell leukemia. In another embodiment, the T-cell leukemia is adult T-cell leukemia. In certain embodiments, the methods of treating, preventing and/or managing ALL in a subject comprise the step of administering to the subject an amount of a formulation of Compound 1 provided herein effective to treat, prevent and/or manage ALL. In some embodiments, the methods comprise the step of administering to the subject a formulation of Compound 1 provided herein in combination with a second active agent in amounts effective to treat, prevent and/or manage ALL.

In some embodiments, the methods provided herein encompass treating, preventing and/or managing chronic myelogenous leukemia (CML) in a subject. The methods comprise the step of administering to the subject an amount of a formulation of Compound 1 provided herein effective to treat, prevent and/or manage CML. In some embodiments, the methods comprise the step of administering to the subject a formulation of Compound 1 provided herein in combination with a second active agent in amounts effective to treat, prevent and/or manage CML.

In some embodiments, the methods provided herein encompass treating, preventing and/or managing chronic lymphocytic leukemia (CLL) in a subject. The methods comprise the step of administering to the subject an amount of a formulation of Compound 1 provided herein effective to treat, prevent and/or manage chronic lymphocytic leukemia. In some embodiments, the methods comprise the step of administering to the subject a formulation of Compound 1 provided herein in combination with a second active agent in amounts effective to treat, prevent and/or manage CLL.

In one embodiment provided herein is a method for achieving a complete remission (CR) in a CLL patient, wherein the method comprises administering an effective amount of a formulation of Compound 1 to the patient. Provided herein is a formulation of Compound 1 for use in a method for achieving a complete remission (CR) in a CLL patient.

In one embodiment provided herein is a method for achieving a complete remission (CR) in a CLL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in a CLL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a complete remission (CR) in a CLL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in a CLL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial remission (PR) in a CLL patient, wherein the method comprises administering an effective amount of a formulation of Compound 1 to the patient. Provided herein is a formulation of Compound 1 for use in a method for achieving a partial remission (PR) in a CLL patient.

In one embodiment provided herein is a method for achieving a partial remission (PR) in a CLL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial remission (PR) in a CLL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

Provided herein is a formulation of Compound 1 for use in a method for achieving a partial remission (PR) in a CLL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a stable disease (SD) in a CLL patient, wherein the method comprises administering an effective amount of a formulation of Compound 1 to the patient. Provided herein is Compound 1 for use in a method for achieving a stable disease (SD) in a CLL patient.

In one embodiment provided herein is a method for achieving a stable disease (SD) in a CLL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a stable disease (SD) in a CLL patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a stable disease (SD) in a CLL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a stable disease (SD) in a CLL patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment, provided herein are methods of treating, preventing, managing, and/or ameliorating a myelodysplastic syndrome (MDS) by administering a therapeutically active amount of a formulation of Compound 1 to a subject. In one embodiment provided herein is a method of treating MDS. Thus, provided herein is a formulation of Compound 1 for use in such methods of treating, preventing, managing, and/or ameliorating MDS. In one embodiment, the MDS is relapsed, resistant or refractory MDS. In one embodiment, MDS is refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms or chronic myelomonocytic leukemia (CMML). In some embodiments, the MDS is very low risk, low risk, intermediate risk, high risk or very high risk MDS. In one embodiment, the MDS is very low risk. In another embodiment, the MDS is low risk. In another embodiment, the MDS is intermediate risk. In another embodiment, the MDS is high risk. In another embodiment, the MDS is very high risk MDS. In one embodiment, the MDS is relapsed or refractory high risk MDS. In one embodiment, the MDS is with a score >3.5 points in the Revised International Prognostic Scoring System (IPSS-R) (eg, IPSS-R intermediate risk (in combination with more than 10% bone marrow blasts or poor or very poor IPSS-R cytogenetic risk), IPSS-R high and IPSS-R very high risk]. In one embodiment, the MDS is not suitable for other established therapies (eg, transplant or hypomethylating agent). In some embodiments, the MDS is primary or de novo MDS. In other embodiments, the MDS is secondary MDS. In one embodiment, the MDS is refractory to initial induction or re-induction treatment. In certain embodiments, the MDS is refractory to at least one induction/reinduction or consolidation therapy.

In one embodiment provided herein is a method for achieving a complete remission (CR) in an MDS patient, wherein the method comprises administering an effective amount of a formulation of Compound 1 to the patient. Provided herein is a formulation of Compound 1 for use in a method for achieving a complete remission (CR) in an MDS patient.

In one embodiment provided herein is a method for achieving a complete remission (CR) in an MDS patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in an MDS patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a complete remission (CR) in an MDS patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a complete remission (CR) in an MDS patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a marrow complete remission (mCR) in an MDS patient, wherein the method comprises administering an effective amount of a formulation of Compound 1 to the patient. Provided herein is a formulation of Compound 1 for use in a method for achieving a marrow complete remission (mCR) in an MDS patient.

In one embodiment provided herein is a method for achieving a marrow complete remission (mCR) in an MDS patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a marrow complete remission (mCR) in an MDS patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a marrow complete remission (mCR) in an MDS patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a marrow complete remission (mCR) in an MDS patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial remission (PR) in an MDS patient, wherein the method comprises administering an effective amount of a formulation of Compound 1 to the patient. Provided herein is a formulation of Compound 1 for use in a method for achieving a partial remission (PR) in an MDS patient.

In one embodiment provided herein is a method for achieving a partial remission (PR) in an MDS patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial remission (PR) in an MDS patient, wherein the method comprises administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment provided herein is a method for achieving a partial remission (PR) in an MDS patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in a method for achieving a partial remission (PR) in an MDS patient, wherein the method comprises administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment, provided herein are methods for increasing overall survival, increasing relapse free survival, increasing progression free survival, increasing event-free survival, increasing duration of remission, increasing duration of response, or increasing time to transformation to AML in an MDS patient, comprising administering an effective amount of a formulation of Compound 1 to the patient. Provided herein is a formulation of Compound 1 for use in methods for increasing overall survival, increasing relapse free survival, increasing progression free survival, increasing event-free survival, increasing duration of remission, increasing duration of response, or increasing time to transformation to AML in an MDS patient.

In one embodiment, provided herein are methods for increasing overall survival, increasing relapse free survival, increasing progression free survival, increasing event-free survival, increasing duration of remission, increasing duration of response, or increasing time to transformation to AML in an MDS patient, comprising administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in methods for increasing overall survival, increasing relapse free survival, increasing progression free survival, increasing event-free survival, increasing duration of remission, increasing duration of response, or increasing time to transformation to AML in an MDS patient, comprising administering an effective amount of a formulation of Compound 1 in combination with one or more second agents selected from glucocorticoid receptor agonists, IL-113 receptor antagonists, interleukin-113 blockers, JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In one embodiment, provided herein are methods for increasing overall survival, increasing relapse free survival, increasing progression free survival, increasing event-free survival, increasing duration of remission, increasing duration of response, or increasing time to transformation to AML in an MDS patient, comprising administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient. Provided herein is a formulation of Compound 1 for use in methods for increasing overall survival, increasing relapse free survival, increasing progression free survival, increasing event-free survival, increasing duration of remission, increasing duration of response, or increasing time to transformation to AML in an MDS patient, comprising administering an effective amount of a formulation of Compound 1 in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors to the patient.

In some embodiments, the methods provided herein encompass treating, preventing and/or managing a myeloproliferative neoplasm. In one embodiment, the myeloproliferative neoplasm is polycythemia vera, primary or essential thrombocythemia, myelofibrosis, chronic myelogenous leukemia, chronic neutrophilic leukemia, juvenile myelomonocytic leukemia, chronic eosinophilic leukemia, or hyper eosinophilic syndrome. In one embodiment, the myeloproliferative neoplasm is polycythemia vera, primary or essential thrombocythemia, primary or idiopathic myelofibrosis, secondary myeolofibrosis, post polycythemia vera myelofibrosis, post essential thrombocythemia myelofibrosis, chronic myelogenous leukemia, chronic neutrophilic leukemia, juvenile myelomonocytic leukemia, chronic eosinophilic leukemia, or hyper eosinophilic syndrome. In one embodiment, the myeloproliferative neoplasm is polycythemia vera. In one embodiment, the myeloproliferative neoplasm is primary or essential thrombocythemia. In one embodiment, the myeloproliferative neoplasm is myelofibrosis. In one embodiment, the myeloproliferative neoplasm is primary or idiopathic myelofibrosis. In one embodiment, the myeloproliferative neoplasm is secondary myeolofibrosis. In one embodiment, the myeloproliferative neoplasm is post polycythemia vera myelofibrosis. In one embodiment, the myeloproliferative neoplasm is post essential thrombocythemia myelofibrosis. In one embodiment, the myeloproliferative neoplasm is chronic myelogenous leukemia. In one embodiment, the myeloproliferative neoplasm is chronic neutrophilic leukemia. In one embodiment, the myeloproliferative neoplasm is juvenile myelomonocytic leukemia. In one embodiment, the myeloproliferative neoplasm is chronic eosinophilic leukemia. In one embodiment, the myeloproliferative neoplasm is hyper eosinophilic syndrome. In certain embodiments, the myeloproliferative neoplasm is interleukin-3 (IL-3) independent. In some embodiments, the myeloproliferative neoplasm is characterized by a JAK mutation, for example, a V617 mutation, such as V617F.

In certain embodiments, the methods of treating, preventing and/or managing a myeloproliferative neoplasm in a subject comprise the step of administering to the subject an amount of a formulation of Compound 1 provided herein effective to treat, prevent and/or manage myeloproliferative neoplasm. In some embodiments, the methods comprise the step of administering to the subject a formulation of Compound 1 provided herein in combination with a second active agent in amounts effective to treat, prevent and/or manage myeloproliferative neoplasm.

In one embodiment, the methods of treating, preventing and/or managing cancer provided herein comprise intravenous administration of a formulation of Compound 1. In one embodiment, the formulation of Compound 1 is dissolved in water to form an aqueous solution for intravenous administration in methods of treating, preventing and/or managing cancer provided herein.

In some embodiments, the methods comprise the step of administering to the subject a formulation of Compound 1 provided herein in combination with a second active agent in amounts effective to treat, prevent and/or manage cancer.

In certain embodiments, provided herein are methods of treating, preventing, and/or managing cancer in patients with impaired renal function. In certain embodiments, provided herein are methods of providing appropriate dose adjustments for patients with impaired renal function due to, but not limited to, disease, aging, or other patient factors.

In certain embodiments, a therapeutically or prophylactically effective amount of Compound 1 is from about 0.005 to about 20 mg per day, from about 0.05 to 20 mg per day, from about 0.01 to about 10 mg per day, from about 0.01 to about 7 mg per day, from about 0.01 to about 5 mg per day, from about 0.01 to about 3 mg per day, from about 0.05 to about 10 mg per day, from about 0.05 to about 7 mg per day, from about 0.05 to about 5 mg per day, from about 0.05 to about 3 mg per day, from about 0.1 to about 15 mg per day, from about 0.1 to about 10 mg per day, from about 0.1 to about 7 mg per day, from about 0.1 to about 5 mg per day, from about 0.1 to about 3 mg per day, from about 0.5 to about 10 mg per day, from about 0.05 to about 5 mg per day, from about 0.5 to about 3 mg per day, from about 0.5 to about 2 mg per day, from about 0.3 to about 10 mg per day, from about 0.3 to about 8.5 mg per day, from about 0.3 to about 8.1 mg per day, from about 0.6 to about 10 mg per day or from about 0.6 to about 5 mg per day. In one embodiment, a therapeutically or prophylactically effective amount of Compound 1 is from about 0.005 to about 20 mg per day. In one embodiment, a therapeutically or prophylactically effective amount of Compound 1 is, from about 0.05 to 20 mg per day. In one embodiment, a therapeutically or prophylactically effective amount of Compound 1 is from about 0.01 to about 10 mg per day. In one embodiment, a therapeutically or prophylactically effective amount of Compound 1 is from about 0.01 to about 7 mg per day. In one embodiment, a therapeutically or prophylactically effective amount of Compound 1 is from about 0.01 to about 5 mg per day. In one embodiment, a therapeutically or prophylactically effective amount of Compound 1 is from about 0.01 to about 3 mg per day. In one embodiment, a therapeutically or prophylactically effective amount of Compound 1 is from about 0.05 to about 10 mg per day. In one embodiment, a therapeutically or prophylactically effective amount of Compound 1 is from about 0.05 to about 7 mg per day. In one embodiment, a therapeutically or prophylactically effective amount of Compound 1 is from about 0.05 to about 5 mg per day. In one embodiment, a therapeutically or prophylactically effective amount of Compound 1 is from about 0.05 to about 3 mg per day. In one embodiment, a therapeutically or prophylactically effective amount of Compound 1 is from about 0.1 to about 15 mg per day. In one embodiment, a therapeutically or prophylactically effective amount of Compound 1 is from about 0.1 to about 10 mg per day. In one embodiment, a therapeutically or prophylactically effective amount of Compound 1 is from about 0.1 to about 7 mg per day. In one embodiment, a therapeutically or prophylactically effective amount of Compound 1 is from about 0.1 to about 5 mg per day. In one embodiment, a therapeutically or prophylactically effective amount of Compound 1 is from about 0.1 to about 3 mg per day. In one embodiment, a therapeutically or prophylactically effective amount of Compound 1 is from about 0.5 to about 10 mg per day. In one embodiment, a therapeutically or prophylactically effective amount of Compound 1 is from about 0.5 to about 5 mg per day. In one embodiment, a therapeutically or prophylactically effective amount of Compound 1 is from about 0.5 to about 3 mg per day. In one embodiment, a therapeutically or prophylactically effective amount of Compound 1 is from about 0.5 to about 2 mg per day. In one embodiment, a therapeutically or prophylactically effective amount of Compound 1 is from about 0.3 to about 10 mg per day. In one embodiment, a therapeutically or prophylactically effective amount of Compound 1 is from about 0.3 to about 8.5 mg per day. In one embodiment, a therapeutically or prophylactically effective amount of Compound 1 is from about 0.3 to about 8.1 mg per day. In one embodiment, a therapeutically or prophylactically effective amount of Compound 1 is from about 0.6 to about 10 mg per day or from about 0.6 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, or about 10 mg per day. In some such embodiments, the therapeutically or prophylactically effective amount is about 0.5, about 0.6, about 0.75, about 1, about 2, about 3, about 4, about 5, about 6 or about 7 mg per day. In some such embodiments, the therapeutically or prophylactically effective amount is about 0.6, about 1.2, about 1.8, about 2.4, about 3, about 3.6 mg or about 4.5 mg per day. In some such embodiments, the therapeutically or prophylactically effective amount is about 0.6, about 1.2, about 1.8, about 2.4, or about 3.6 mg per day. In certain embodiments, the therapeutically or prophylactically effective amount is about 0.1 mg per day. In certain embodiments, the therapeutically or prophylactically effective amount is about 0.2 mg per day. In certain embodiments, the therapeutically or prophylactically effective amount is about 0.5 mg per day. In certain embodiments, the therapeutically or prophylactically effective amount is about about 1 mg per day. In certain embodiments, the therapeutically or prophylactically effective amount is about about 2 mg per day. In certain embodiments, the therapeutically or prophylactically effective amount is about about 3 mg per day. In certain embodiments, the therapeutically or prophylactically effective amount is about about 4 mg per day. In certain embodiments, the therapeutically or prophylactically effective amount is about about 4.5 mg per day. In certain embodiments, the therapeutically or prophylactically effective amount is about about 5 mg per day. In certain embodiments, the therapeutically or prophylactically effective amount is about about 6 mg per day. In certain embodiments, the therapeutically or prophylactically effective amount is about about 7 mg per day. In certain embodiments, the therapeutically or prophylactically effective amount is about about 8 mg per day. In certain embodiments, the therapeutically or prophylactically effective amount is about about 9 mg per day. In certain embodiments, the therapeutically or prophylactically effective amount is about about 10 mg per day.

In one embodiment, the recommended daily dose range of Compound 1, for the conditions described herein lie within the range of from about 0.01 mg to about 20 mg per day, preferably given as a single once-a-day dose, or in divided doses throughout a day. In one embodiment, the recommended daily dose range of Compound 1, for the conditions described herein lie within the range of from about 0.01 mg to about 15 mg per day, preferably given as a single once-a-day dose, or in divided doses throughout a day. In one embodiment, the recommended daily dose range of Compound 1, for the conditions described herein lie within the range of from about 0.01 mg to about 12 mg per day, preferably given as a single once-a-day dose, or in divided doses throughout a day. In some embodiments, the dosage ranges from about 0.1 mg to about 10 mg per day. In other embodiments, the dosage ranges from about 0.5 to about 5 mg per day. Specific doses per day include 0.1, 0.2, 0.5, 0.6, 1, 1.2, 1.5, 1.8, 2, 2.4, 2.5, 3, 3.5, 3.6, 4, 4.5, 5, 5.5, 6, 6.5, 7, 7.2, 7.5, 8, 8.5, 9, 9.5, 10, 10.5, 11, 11.5, 12, 12.5, 13, 13.5, 14, 14.4, 14.5 or 15 mg per day. In other embodiments, the dosage ranges from about 0.5 to about 5 mg per day. Specific doses per day include 0.1, 0.2, 0.5, 0.6, 1, 1.2, 1.5, 1.8, 2, 2.4, 2.5, 3, 3.5, 3.6, 4, 4.5, 5, 5.5, 6, 6.5, 7, 7.5, 8, 8.5, 9, 9.5 or 10 mg per day. In one embodiment, the dose per day is 0.1 mg per day. In one embodiment, the dose per day is 0.2 mg per day. In one embodiment, the dose per day is 0.5 mg per day. In one embodiment, the dose per day is 0.6 mg per day. In one embodiment, the dose per day is 1 mg per day. In one embodiment, the dose per day is 1.2 mg per day. In one embodiment, the dose per day is 1.5 mg per day. In one embodiment, the dose per day is 1.8 mg per day. In one embodiment, the dose per day is 2 mg per day. In one embodiment, the dose per day is 2.4 mg per day. In one embodiment, the dose per day is 2.5 mg per day. In one embodiment, the dose per day is 3 mg per day. In one embodiment, the dose per day is 3.5 mg per day. In one embodiment, the dose per day is 3.6 mg per day. In one embodiment, the dose per day is 4 mg per day. In one embodiment, the dose per day is 4.5 mg per day. In one embodiment, the dose per day is 5 mg per day. In one embodiment, the dose per day is 5.5 mg per day. In one embodiment, the dose per day is 6 mg per day. In one embodiment, the dose per day is 6.5 mg per day. In one embodiment, the dose per day is 7 mg per day. In one embodiment, the dose per day is 7.2 mg per day. In one embodiment, the dose per day is 7.5 mg per day. In one embodiment, the dose per day is 8 mg per day. In one embodiment, the dose per day is 8.5 mg per day. In one embodiment, the dose per day is 9 mg per day. In one embodiment, the dose per day is 9.5 mg per day. In one embodiment, the dose per day is 10 mg per day. In one embodiment, the dose per day is 12 mg per day. In one embodiment, the dose per day is 10 mg per day. In one embodiment, the dose per day is 12 mg per day. In one embodiment, the dose per day is 14.4 mg per day. In one embodiment, the dose per day is 15 mg per day.

In a specific embodiment, the recommended starting dosage may be 0.1, 0.5, 0.6, 0.7, 1, 1.2, 1.5, 1.8, 2, 2.4, 2.5, 3, 3.5, 3.6, 4, 4.5, 5, 5.5, 6, 6.5 or 7 mg per day. In another embodiment, the recommended starting dosage may be 0.1, 0.5, 0.6, 1, 1.2, 1.8, 2, 2.4, 3, 3.6, 4, 4.5, or 5 mg per day. In another embodiment, the recommended starting dosage may be 0.1, 0.5, 0.6, 1, 1.2, 1.8, 2, 2.4, 3, 3.6, 4, or 5 mg per day. In one embodiment, the dose may be escalated to 7, 8, 9 10, 12, or 15 mg/day. In one embodiment, the dose may be escalated to 7, 8, 9 or 10 mg/day.

In a specific embodiment, Compound 1 can be administered in an amount of about 0.1 mg/day to patients with leukemia, including AML. In a particular embodiment, Compound 1 can be administered in an amount of about 1 mg/day to patients with leukemia, including AML. In a particular embodiment, Compound 1 can be administered in an amount of about 3 mg/day to patients with leukemia, including AML. In a particular embodiment, Compound 1 can be administered in an amount of about 3.6 mg/day to patients with leukemia, including AML. In a particular embodiment, Compound 1 can be administered in an amount of about 4 mg/day to patients with leukemia, including AML. In a particular embodiment, Compound 1 can be administered in an amount of about 4.5 mg/day to patients with leukemia, including AML. In a particular embodiment, Compound 1 provided herein can be administered in an amount of about 5 mg/day to patients with leukemia, including AML. In a particular embodiment, Compound 1 provided herein can be administered in an amount of about 6 mg/day to patients with leukemia, including AML. In a particular embodiment, Compound 1 provided herein can be administered in an amount of about 7 mg/day to patients with leukemia, including AML. In a particular embodiment, Compound 1 provided herein can be administered in an amount of about 10 mg/day to patients with leukemia, including AML. In a particular embodiment, Compound 1 provided herein can be administered in an amount of about 12 mg/day to patients with leukemia, including AML. In a particular embodiment, Compound 1 provided herein can be administered in an amount of about 15 mg/day to patients with leukemia, including AML.

In a specific embodiment, Compound 1 can be administered in an amount of about 0.1 mg/day to patients with MDS. In a particular embodiment, Compound 1 can be administered in an amount of about 1 mg/day to patients with MDS. In a particular embodiment, Compound 1 can be administered in an amount of about 3 mg/day to patients with MDS. In a particular embodiment, Compound 1 can be administered in an amount of about 3.6 mg/day to patients with MDS. In a particular embodiment, Compound 1 can be administered in an amount of about 4 mg/day to patients with MDS. In a particular embodiment, Compound 1 can be administered in an amount of about 4.5 mg/day to patients with MDS. In a particular embodiment, Compound 1 provided herein can be administered in an amount of about 5 mg/day to patients with MDS. In a particular embodiment, Compound 1 provided herein can be administered in an amount of about 6 mg/day to patients with MDS. In a particular embodiment, Compound 1 provided herein can be administered in an amount of about 7 mg/day to patients with MDS. In a particular embodiment, Compound 1 provided herein can be administered in an amount of about 10 mg/day to patients with MDS. In a particular embodiment, Compound 1 provided herein can be administered in an amount of about 12 mg/day to patients with MDS. In a particular embodiment, Compound 1 provided herein can be administered in an amount of about 15 mg/day to patients with MDS.

In certain embodiments, the therapeutically or prophylactically effective amount is from about 0.001 to about 20 mg/kg/day, from about 0.01 to about 15 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day. In certain embodiments, the therapeutically or prophylactically effective amount is from about 0.001 to about 20 mg/kg/day. In certain embodiments, the therapeutically or prophylactically effective amount is from about 0.01 to about 15 mg/kg/day. In certain embodiments, the therapeutically or prophylactically effective amount is from about 0.01 to about 10 mg/kg/day. In certain embodiments, the therapeutically or prophylactically effective amount is from about 0.01 to about 9 mg/kg/day. In certain embodiments, the therapeutically or prophylactically effective amount is 0.01 to about 8 mg/kg/day. In certain embodiments, the therapeutically or prophylactically effective amount is from about 0.01 to about 7 mg/kg/day. In certain embodiments, the therapeutically or prophylactically effective amount is from about 0.01 to about 6 mg/kg/day. In certain embodiments, the therapeutically or prophylactically effective amount is from about 0.01 to about 5 mg/kg/day. In certain embodiments, the therapeutically or prophylactically effective amount is from about 0.01 to about 4 mg/kg/day. In certain embodiments, the therapeutically or prophylactically effective amount is from about 0.01 to about 3 mg/kg/day. In certain embodiments, the therapeutically or prophylactically effective amount is from about 0.01 to about 2 mg/kg/day. In certain embodiments, the therapeutically or prophylactically effective amount is from about 0.01 to about 1 mg/kg/day. In certain embodiments, the therapeutically or prophylactically effective amount is from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In other embodiments, the amount of a formulation of Compound 1 administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM. In other embodiments, the amount of a formulation of Compound 1 administered is sufficient to provide a plasma concentration of the compound at steady state, ranging from about 5 to about 100 nM. In other embodiments, the amount of a formulation of Compound 1 administered is sufficient to provide a plasma concentration of the compound at steady state, ranging from about 5 to about 50 nM. In other embodiments, the amount of a formulation of Compound 1 administered is sufficient to provide a plasma concentration of the compound at steady state, ranging from about 10 to about 100 nM. In other embodiments, the amount of a formulation of Compound 1 administered is sufficient to provide a plasma concentration of the compound at steady state, ranging from about 10 to about 50 nM. In other embodiments, the amount of a formulation of Compound 1 administered is sufficient to provide a plasma concentration of the compound at steady state, ranging from about 50 to about 100 nM.

As used herein, the term “plasma concentration at steady state” is the concentration reached after a period of administration of a formulation provided herein. Once steady state is reached, there are minor peaks and troughs on the time dependent curve of the plasma concentration of the solid form.

In certain embodiments, the amount of a formulation of Compound 1 administered is sufficient to provide a maximum plasma concentration (peak concentration) of the compound, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM. In certain embodiments, the amount of a formulation of Compound 1 administered is sufficient to provide a maximum plasma concentration (peak concentration) of the compound, ranging from about 0.001 to about 500 μM. In certain embodiments, the amount of a formulation of Compound 1 administered is sufficient to provide a maximum plasma concentration (peak concentration) of the compound, ranging from about 0.002 to about 200 μM. In certain embodiments, the amount of a formulation of Compound 1 administered is sufficient to provide a maximum plasma concentration (peak concentration) of the compound, ranging from about 0.005 to about 100 μM. In certain embodiments, the amount of a formulation of Compound 1 administered is sufficient to provide a maximum plasma concentration (peak concentration) of the compound, ranging from about 0.01 to about 50 μM. In certain embodiments, the amount of a formulation of Compound 1 administered is sufficient to provide a maximum plasma concentration (peak concentration) of the compound, ranging from about 1 to about 50 μM. In certain embodiments, the amount of a formulation of Compound 1 administered is sufficient to provide a maximum plasma concentration (peak concentration) of the compound, ranging from about 0.02 to about 25 μM. In certain embodiments, the amount of a formulation of Compound 1 administered is sufficient to provide a maximum plasma concentration (peak concentration) of the compound, ranging from about 0.05 to about 20 μM. In certain embodiments, the amount of a formulation of Compound 1 administered is sufficient to provide a maximum plasma concentration (peak concentration) of the compound, ranging from about 0.1 to about 20 μM. In certain embodiments, the amount of a formulation of Compound 1 administered is sufficient to provide a maximum plasma concentration (peak concentration) of the compound, ranging from about 0.5 to about 20 μM. In certain embodiments, the amount of a formulation of Compound 1 administered is sufficient to provide a maximum plasma concentration (peak concentration) of the compound, ranging from about 1 to about 20 μM.

In certain embodiments, the amount of a formulation of Compound 1 administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM. In certain embodiments, the amount of a formulation of Compound 1 administered is sufficient to provide a minimum plasma concentration (trough concentration) of the compound, ranging from about 0.001 to about 500 μM. In certain embodiments, the amount of a formulation of Compound 1 administered is sufficient to provide a minimum plasma concentration (trough concentration) of the compound, ranging from about 0.002 to about 200 μM. In certain embodiments, the amount of a formulation of Compound 1 administered is sufficient to provide a minimum plasma concentration (trough concentration) of the compound, ranging from about 0.005 to about 100 μM. In certain embodiments, the amount of a formulation of Compound 1 administered is sufficient to provide a minimum plasma concentration (trough concentration) of the compound, ranging from about 0.01 to about 50 μM. In certain embodiments, the amount of a formulation of Compound 1 administered is sufficient to provide a minimum plasma concentration (trough concentration) of the compound, ranging from about 1 to about 50 μM, about 0.01 to about 25 μM. In certain embodiments, the amount of a formulation of Compound 1 administered is sufficient to provide a minimum plasma concentration (trough concentration) of the compound, ranging from about 0.01 to about 20 μM. In certain embodiments, the amount of a formulation of Compound 1 administered is sufficient to provide a minimum plasma concentration (trough concentration) of the compound, ranging from about 0.02 to about 20 μM. In certain embodiments, the amount of a formulation of Compound 1 administered is sufficient to provide a minimum plasma concentration (trough concentration) of the compound, ranging from about 0.02 to about 20 μM. In certain embodiments, the amount of a formulation of Compound 1 administered is sufficient to provide a minimum plasma concentration (trough concentration) of the compound, ranging from about 0.01 to about 20 μM.

In certain embodiments, the amount of a formulation of Compound 1 administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL. In certain embodiments, the amount of a formulation of Compound 1 administered is sufficient to provide an area under the curve (AUC) of the compound, ranging from about 100 to about 100,000 ng*hr/mL. In certain embodiments, the amount of a formulation of Compound 1 administered is sufficient to provide an area under the curve (AUC) of the compound, ranging from about 1,000 to about 50,000 ng*hr/mL. In certain embodiments, the amount of a formulation of Compound 1 administered is sufficient to provide an area under the curve (AUC) of the compound, ranging from about 5,000 to about 25,000 ng*hr/mL. In certain embodiments, the amount of a formulation of Compound 1 administered is sufficient to provide an area under the curve (AUC) of the compound, ranging from about 5,000 to about 10,000 ng*hr/mL.

In certain embodiments, the patient to be treated with one of the methods provided herein has not been treated with anti-cancer therapy prior to the administration of a formulation of Compound 1 provided herein. In certain embodiments, the patient to be treated with one of the methods provided herein has been treated with anti-cancer therapy prior to the administration of a formulation of Compound 1 provided herein. In certain embodiments, the patient to be treated with one of the methods provided herein has developed drug resistance to the anti-cancer therapy.

The methods provided herein encompass treating a patient regardless of patient's age, although some diseases or disorders are more common in certain age groups.

The formulation of Compound 1 provided herein can be delivered as a single dose such as, e.g., a single bolus injection, or over time, such as, e.g., continuous infusion over time or divided bolus doses over time. The formulation of Compound 1 can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The formulation of Compound 1 provided herein can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of at least 10 days to 52 weeks. The term “intermittent” or “intermittently” as used herein is intended to mean stopping and starting at either regular or irregular intervals. For example, intermittent administration of the formulation of Compound 1 is administration for one to six days per week, administration in cycles (e.g., daily administration for one to ten consecutive days of a 28 day cycle, then a rest period with no administration for rest of the 28 day cycle; or daily administration for two to eight consecutive weeks, then a rest period with no administration for up to one week), or administration on alternate days. Cycling therapy with Compound 1 is discussed elsewhere herein.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the formulation of Compound 1 is administered once a day. In another embodiment, the formulation of Compound 1 is administered twice a day. In yet another embodiment, the formulation of Compound 1 provided herein is administered three times a day. In still another embodiment, the formulation of Compound 1 provided herein is administered four times a day. In still another embodiment, the formulation of Compound 1 provided herein is administered once every other day. In still another embodiment, the formulation of Compound 1 provided herein is administered twice a week. In still another embodiment, the formulation of Compound 1 provided herein is administered once every week. In still another embodiment, the formulation of Compound 1 provided herein is administered once every two weeks. In still another embodiment, the formulation of Compound 1 provided herein is administered once every three weeks. In still another embodiment, Compound 1 provided herein is administered once every four weeks.

In certain embodiments, a formulation of Compound 1 provided herein is administered once per day from one day to six months, from one week to three months, from one week to four weeks, from one week to three weeks, or from one week to two weeks. In certain embodiments, a formulation of Compound 1 provided herein is administered once per day for one week, two weeks, three weeks, or four weeks. In one embodiment, a formulation of Compound 1 provided herein is administered once per day for 1 day. In one embodiment, a formulation of Compound 1 provided herein is administered once per day for 2 days. In one embodiment, a formulation of Compound 1 provided herein is administered once per day for 3 days. In one embodiment, a formulation of Compound 1 provided herein is administered once per day for 4 days. In one embodiment, a formulation of Compound 1 provided herein is administered once per day for 5 days. In one embodiment, a formulation of Compound 1 provided herein is administered once per day for 6 days. In one embodiment, a formulation of Compound 1 provided herein is administered once per day for one week. In one embodiment, a formulation of Compound 1 provided herein is administered once per day for up to 10 days. In another embodiment, a formulation of Compound 1 provided herein is administered once per day for two weeks. In yet another embodiment, a formulation of Compound 1 provided herein is administered once per day for three weeks. In still another embodiment, a formulation of Compound 1 provided herein is administered once per day for four weeks.

Combination Therapy

In one embodiment, provided herein is a method of treating, preventing, and/or managing cancer, comprising administering to a patient a formulation of Compound 1 provided herein in combination with one or more second active agents, and optionally in combination with radiation therapy, blood transfusions, or surgery. Examples of second active agents are disclosed herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. E.g., “in combination” may include administration as a mixture, simultaneous administration using separate formulations, and consecutive administration in any order. “Consecutive” means that a specific time has passed between the administration of the active agents. For example, “consecutive” may be that more than 10 minutes have passed between the administration of the separate active agents. The time period can then be more than 10 min, more than 30 minutes, more than 1 hour, more than 3 hours, more than 6 hours or more than 12 hours. E.g., a first therapy (e.g., a prophylactic or therapeutic agent such as a formulation of Compound 1 provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

In one embodiment, administration of a formulation of Compound 1 provided herein, and one or more second active agents to a patient can occur simultaneously or sequentially by the same or different routes of administration. In one embodiment, administration of a formulation of Compound 1 provided herein, and one or more second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated.

The route of administration of a formulation of Compound 1 provided herein, is independent of the route of administration of a second therapy. Thus, in one embodiment, a formulation of Compound 1 provided herein, is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, a formulation of Compound 1 provided herein, and a second therapy are administered by the same mode of administration, by IV. In another embodiment, a formulation of Compound 1 provided herein, is administered by one mode of administration, e.g., by IV, whereas the second agent (an anti-cancer agent) is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated and/or managed, the severity and stage of disease, and the amount of Compound 1 and any optional additional active agents concurrently administered to the patient.

One or more second active ingredients or agents can be used together with Compound 1 in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins. Proteins that are particularly useful in the methods and compositions provided herein include proteins that stimulate the survival and/or proliferation of hematopoietic precursor cells and immunologically active poietic cells in vitro or in vivo. Other useful proteins stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

In certain embodiments, GM-CSF, G-CSF, SCF or EPO is administered subcutaneously during about five days in a four or six week cycle in an amount ranging from about 1 to about 750 mg/m²/day, from about 25 to about 500 mg/m²/day, from about 50 to about 250 mg/m²/day, or from about 50 to about 200 mg/m²/day. In certain embodiments, GM-CSF may be administered in an amount of from about 60 to about 500 mcg/m² intravenously over 2 hours or from about 5 to about 12 mcg/m²/day subcutaneously. In certain embodiments, G-CSF may be administered subcutaneously in an amount of about 1 mcg/kg/day initially and can be adjusted depending on rise of total granulocyte counts. The maintenance dose of G-CSF may be administered in an amount of about 300 (in smaller patients) or 480 mcg subcutaneously. In certain embodiments, EPO may be administered subcutaneously in an amount of 10,000 Unit 3 times per week.

Particular proteins that can be used in the methods and compositions include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; the entireties of which are incorporated herein by reference.

Also provided for use in combination with a formulation of Compound 1, are native, naturally occurring, and recombinant proteins. Further encompassed are mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with a formulation of Compound 1 provided herein, include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar®), edrecolomab (Panorex®), and G250. The formulation of Compound 1 can also be combined with, or used in combination with, anti-TNF-α antibodies, and/or anti-EGFR antibodies, such as, for example, Erbitux® or panitumumab.

Large molecule active agents may be administered in the form of anti-cancer vaccines. For example, vaccines that secrete, or cause the secretion of, cytokines such as IL-2, G-CSF, and GM-CSF can be used in the methods and pharmaceutical compositions provided. See, e.g., Emens, L. A., et al., Curr. Opinion Mol. Ther. 3(1):77-84 (2001).

Second active agents that are small molecules can also be used to alleviate adverse effects associated with the administration of a formulation of Compound 1 provided herein. However, like some large molecules, many are believed to be capable of providing a synergistic effect when administered with (e.g., before, after, or simultaneously) a formulation of Compound 1 provided herein. Examples of small molecule second active agents include, but are not limited to, anti-cancer agents, antibiotics, immunosuppressive agents, and steroids.

In certain embodiments, the second agent is an HSP inhibitor, a proteasome inhibitor, a FLT3 inhibitor or an mTOR inhibitor. In some embodiments, the mTOR inhibitor is a mTOR kinase inhibitor.

Examples of anti-cancer agents to be used within the methods or compositions described herein include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; clofarabine; crisnatol mesylate; cyclophosphamide; Ara-C; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; omacetaxine; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sorafenib; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs to be included within the methods herein include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; Ara-C ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®); imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; j asplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosane polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In one embodiment, the second active agent is a glucocorticoid receptor agonist, for example, prednisone, prednisolone, methylpredisolone, hydrocortisone, cortisol, triamcinolone, betamethasone or dexamethasone. In one embodiment, the second active agent is an IL-113 receptor antagonist, for example anakinra. In one embodiment, the second active agent is an interleukin-113 blocker, for example, canakinumab.

In certain embodiments, the second agent is selected from one or more checkpoint inhibitors. In one embodiment, one checkpoint inhibitor is used in combination with a formulation of Compound 1 in the methods provided herein. In another embodiment, two checkpoint inhibitors are used in combination with a formulation of Compound 1 in connection with the methods provided herein. In yet another embodiment, three or more checkpoint inhibitors are used in combination with a formulation of Compound 1 in connection with the methods provided herein.

As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1 106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitor is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitor is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD 137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhTL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with second active agents as described herein where appropriate for treating diseases described herein and understood in the art.

In certain embodiments, a formulation of Compound 1 provided herein can be used in combination with one or more immune cells expressing one or more chimeric antigen receptors (CARs) on their surface (e.g., a modified immune cell). Generally, CARs comprise an extracellular domain from a first protein e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

Extracellular domains: The extracellular domains of the CARs bind to an antigen of interest. In certain embodiments, the extracellular domain of the CAR comprises a receptor, or a portion of a receptor, that binds to said antigen. In certain embodiments, the extracellular domain comprises, or is, an antibody or an antigen-binding portion thereof. In specific embodiments, the extracellular domain comprises, or is, a single chain Fv (scFv) domain. The single-chain Fv domain can comprise, for example, a V_{L} linked to V_{H} by a flexible linker, wherein said V_{L} and V_{H} are from an antibody that binds said antigen.

In certain embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAPI (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In certain other embodiments, the TAA or TSA recognized by the extracellular domain of a CAR is integrin αvβ3 (CD61), galactin, or Ral-B.

In certain embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXBI, SPA17, SSX, SYCPI, or TPTE.

In certain other embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In certain other embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAA0205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel17), tyrosinase, TRP-1, TRP-2, MAGE-1, MAGE-3, RAGE, GAGE-1, GAGE-2, p15(58), RAGE, SCP-1, Hom/Mel-40, PRAME, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, or TPS.

In various specific embodiments, the tumor-associated antigen or tumor-specific antigen is an AML-related tumor antigen, as described in S. Anguille et al, Leukemia (2012), 26, 2186-2196.

Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

Transmembrane domain: In certain embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein, and can include all or a portion of such transmembrane domain. In specific embodiments, the transmembrane domain can be obtained or derived from, e.g., CD8, CD 16, a cytokine receptor, and interleukin receptor, or a growth factor receptor, or the like.

Intracellular signaling domains: In certain embodiments, the intracellular domain of a CAR is or comprises an intracellular domain or motif of a protein that is expressed on the surface of T cells and triggers activation and/or proliferation of said T cells. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3I) or ITAM-containing portions thereof. In a specific embodiment, the intracellular domain is a CD3I intracellular signaling domain. In other specific embodiments, the intracellular domain is from a lymphocyte receptor chain, a TCR/CD3 complex protein, an Fe receptor subunit or an IL-2 receptor subunit. In certain embodiments, the CAR additionally comprises one or more co-stimulatory domains or motifs, e.g., as part of the intracellular domain of the polypeptide. The one or more co-stimulatory domains or motifs can be, or can comprise comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

The modified immune cells expressing the CARs can be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naive T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR can be isolated using art-accepted, routine methods, e.g., blood collection followed by apheresis and optionally antibody-mediated cell isolation or sorting.

The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes, e.g., unmodified T lymphocytes, or T lymphocytes expressing CD3 and CD28, or comprising a polypeptide comprising a CD3ζ signaling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified immune cells, e.g., modified T lymphocytes, can optionally comprise a “suicide gene” or “safety switch” that enables killing of substantially all of the modified immune cells when desired. For example, the modified T lymphocytes, in certain embodiments, can comprise an HSV thymidine kinase gene (HSV-TK), which causes death of the modified T lymphocytes upon contact with gancyclovir. In another embodiment, the modified T lymphocytes comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 105(11):4247-4254 (2005).

Specific second active agents useful in the methods or compositions include, but are not limited to, rituximab, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, interferon alpha, pegylated interferon alpha (e.g., PEG INTRON-A), capecitabine, cisplatin, thiotepa, fludarabine, carboplatin, liposomal daunorubicin, Ara-C, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ABRAXANE®, ganciclovir, adriamycin, estramustine sodium phosphate (Emcyt®), sulindac, and etoposide. In certain embodiments, the second active agent is ABRAXANE®.

In certain embodiments of the methods provided herein, use of a second active agent in combination with a formulation of Compound 1 provided herein, may be modified or delayed during or shortly following administration of a formulation of Compound 1 provided herein, as deemed appropriate by the practitioner of skill in the art. In certain embodiments, subjects being administered a formulation of Compound 1 provided herein, alone or in combination with other therapies may receive supportive care including antiemetics, myeloid growth factors, and transfusions of platelets, when appropriate. In some embodiments, subjects being administered a formulation of Compound 1 provided herein, may be administered a growth factor as a second active agent according to the judgment of the practitioner of skill in the art. In some embodiments, provided is administration of a formulation of Compound 1 provided herein, in combination with erythropoietin or darbepoetin (Aranesp).

In one aspect, provided herein is a method of treating, preventing, managing, and/or ameliorating locally advanced or metastatic transitional cell bladder cancer comprising administering a formulation of Compound 1 with gemcitabine, cisplatinum, 5-fluorouracil, mitomycin, methotrexate, vinblastine, doxorubicin, carboplatin, thiotepa, paclitaxel, docetaxel, atezolizumab, avelumab, durvalumab, keytruda (pembrolizumab) and/or nivolumab.

In one aspect, methods of treating, preventing, managing, and/or ameliorating a cancer provided herein comprise administering a formulation of Compound 1 in combination with a second active ingredient as follows: temozolomide to pediatric patients with relapsed or progressive brain tumors or recurrent neuroblastoma; celecoxib, etoposide and cyclophosphamide for relapsed or progressive CNS cancer; temodar to patients with recurrent or progressive meningioma, malignant meningioma, hemangiopericytoma, multiple brain metastases, relapsed brain tumors, or newly diagnosed glioblastoma multiforms; irinotecan to patients with recurrent glioblastoma; carboplatin to pediatric patients with brain stem glioma; procarbazine to pediatric patients with progressive malignant gliomas; cyclophosphamide to patients with poor prognosis malignant brain tumors, newly diagnosed or recurrent glioblastoma multiforms; Gliadel® for high grade recurrent malignant gliomas; temozolomide and tamoxifen for anaplastic astrocytoma; or topotecan for gliomas, glioblastoma, anaplastic astrocytoma or anaplastic oligodendroglioma.

In one aspect, methods of treating, preventing, managing, and/or ameliorating a metastatic breast cancer provided herein comprise administering a formulation of Compound 1 with methotrexate, cyclophosphamide, capecitabine, 5-fluorouracil, taxane, temsirolimus, ABRAXANE® (paclitaxel protein-bound particles for injectable suspension) (albumin-bound), lapatinib, herceptin, pamidronate disodium, eribulin mesylate, everolimus, gemcitabine, palbociclib, ixabepilone, kadcyla, pertuzumab, theotepa, anastrozole, docetaxel, doxorubicin hydrochloride, epirubicin hydrochloride, toremifene, fulvestrant, goserelin acetate, ribociclib, megestrol acetate, vinblastin, aromatase inhibitors, such as letrozole, exemestane, selective estrogen modulators, estrogen receptor antagonists, anthracyclines, emtansine, and/or pexidartinib to patients with metastatic breast cancer. In one embodiment, methods of treating, preventing, managing, and/or ameliorating a metastatic breast cancer comprise administering a formulation of Compound 1 with ABRAXANE® to patients with metastatic breast cancer.

In one aspect, methods of treating, preventing, managing, and/or ameliorating neuroendocrine tumors provided herein comprise administering a formulation of Compound 1 with at least one of everolimus, avelumab, sunitinib, nexavar, leucovorin, oxaliplatin, temozolomide, capecitabine, bevacizumab, doxorubicin (Adriamycin), fluorouracil (Adrucil, 5-fluorouracil), streptozocin (Zanosar), dacarbazine, sandostatin, lanreotide, and/or pasireotide to patients with neuroendocrine tumors.

In one aspect, methods of treating, preventing, managing, and/or ameliorating a metastatic breast cancer provided herein comprise administering a formulation of Compound 1 with methotrexate, gemcitabine, cisplatin, cetuximab, 5-fluorouracil, bleomycin, docetaxel, carboplatin, hydroxyurea, pembrolizumab and/or nivolumab to patients with recurrent or metastatic head or neck cancer.

In one aspect, methods of treating, preventing, managing, and/or ameliorating a pancreatic cancer provided herein comprise administering a formulation of Compound 1 with gemcitabine, ABRAXANE®, 5-fluorouracil, afinitor, irinotecan, mitomycin C, sunitinib, sunitinibmalate, and/or tarceva to patients with pancreatic cancer. In one embodiment, methods of treating, preventing, managing, and/or ameliorating a pancreatic cancer comprise administering a formulation of Compound 1 with ABRAXANE® and gemcitabine to patients with pancreatic cancer.

In one aspect, methods of treating, preventing, managing, and/or ameliorating a colon or rectal cancer provided herein comprise administering a formulation of Compound 1 with ARISA®, avastatin, oxaliplatin, 5-fluorouracil, irinotecan, capecitabine, cetuximab, ramucirumab, panitumumab, bevacizumab, leucovorin calcium, lonsurf, regorafenib, ziv-aflibercept, taxol, and/or taxotere.

In one aspect, methods of treating, preventing, managing, and/or ameliorating a refractory colorectal cancer provided herein comprise administering a formulation of Compound 1 with capecitabine and/or vemurafenib to patients with refractory colorectal cancer, or patients who fail first line therapy or have poor performance in colon or rectal adenocarcinoma.

In one aspect, methods of treating, preventing, managing, and/or ameliorating a colorectal cancer provided herein comprise administering a formulation of Compound 1 with fluorouracil, leucovorin, and/or irinotecan to patients with colorectal cancer, including stage 3 and stage 4, or to patients who have been previously treated for metastatic colorectal cancer.

In certain embodiments, a formulation of Compound 1 provided herein is administered to patients with refractory colorectal cancer in combination with capecitabine, xeloda, and/or irinotecan.

In certain embodiments, a formulation of Compound 1 provided herein is administered with capecitabine and irinotecan to patients with refractory colorectal cancer or to patients with unresectable or metastatic colorectal carcinoma.

In one aspect, the methods provided herein comprise administering a formulation of Compound 1 with interferon alpha or capecitabine to patients with unresectable or metastatic hepatocellular carcinoma; or with cisplatin and thiotepa, or with sorafenib tosylate to patients with primary or metastatic liver cancer.

In one aspect, the methods provided herein comprise administering a formulation of Compound 1 with doxorubicin, paclitaxel, vinblastine, pegylated interferon alpha and/or recombinant interferon alpha-2b to patients with Kaposi's sarcoma.

In one aspect, the methods provided herein comprise administering a formulation of Compound 1 with at least one of enasidenib, arsenic trioxide, fludarabine, carboplatin, daunorubicin, cyclophosphamide, cytarabine, doxorubicin, idarubicin, mitoxantrone hydrochloride, thioguanine, vincristine, midostaurin and/or topotecan to patients with acute myeloid leukemia, including refractory or relapsed or high-risk acute myeloid leukemia.

In one aspect, the methods provided herein comprise administering a formulation of Compound 1 with at least one of enasidenib, liposomal daunorubicin, topotecan and/or cytarabine to patients with unfavorable karyotype acute myeloblastic leukemia.

In one aspect, the methods provided herein comprise administering a formulation of Compound 1 with an IDH2 inhibitor to a patient having leukemia, wherein the leukemia is characterized by the presence of a mutant allele of IDH2. Exemplary IDH2 inhibitors are disclosed in U.S. Pat. Nos. 9,732,062; 9,724,350; 9,738,625; and 9,579,324; 10,017,495 and 10,376,510. In one aspect, the methods provided herein comprise administering a formulation of Compound 1 with enasidenib to a patient having leukemia, wherein the leukemia is characterized by the presence of a mutant allele of IDH2. In certain embodiments, the combination of Compound 1 and an IDH2 inhibitor increases differentiated cells (CD34-/CD38) and erythroblasts in a patient having acute myeloid leukemia, wherein the acute myeloid leukemia is characterized by the presence of IDH2 R140Q. In certain embodiments, the combination of a formulation of Compound 1 and an IDH2 inhibitor reduces progenitor cells (CD34+/CD38+) and HSC in a patient having acute myeloid leukemia, wherein the acute myeloid leukemia is characterized by the presence of IDH2 R140Q.

In one aspect, the methods provided herein comprise administering a formulation of Compound 1 with enasidenib to a patient having leukemia, wherein the leukemia is characterized by the presence of a mutant allele of IDH2. In one aspect, the methods provided herein comprise administering a formulation of Compound 1 with enasidenib to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering a formulation of Compound 1 with 6-(6-(trifluoromethyl)pyridin-2-yl)-N²-(2-(trifluoromethyl)pyridin-4-yl)-1,3,5-triazine-2,4-diamine to a patient having leukemia, wherein the leukemia is characterized by the presence of a mutant allele of IDH2. In one aspect, the methods provided herein comprise administering a formulation of Compound 1 with 6-(6-(trifluoromethyl)pyridin-2-yl)-N²-(2-(trifluoromethyl)pyridin-4-yl)-1,3,5-triazine-2,4-diamine to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering a formulation of Compound 1 with methotrexate, mechlorethamine hydrochloride, afatinib dimaleate, pemetrexed, bevacizumab, carboplatin, cisplatin, ceritinib, crizotinib, ramucirumab, pembrolizumab, docetaxel, vinorelbine tartrate, gemcitabine, ABRAXANE®, erlotinib, geftinib, irinotecan, everolimus, alectinib, brigatinib, nivolumab, osimertinib, atezolizumab and/or necitumumab to patients with non-small cell lung cancer. In one embodiment, the methods provided herein comprise administering a formulation of Compound 1 with ABRAXANE® and carboplatin to patients with non-small cell lung cancer.

In one aspect, the methods provided herein comprise administering a formulation of Compound 1 with carboplatin and irinotecan to patients with non-small cell lung cancer.

In one aspect, the methods provided herein comprise administering a formulation of Compound 1 with doxetaxol to patients with non-small cell lung cancer who have been previously treated with carbo/etoposide and radiotherapy.

In one aspect, the methods provided herein comprise administering a formulation of Compound 1 with carboplatin and/or taxotere, or in combination with carboplatin, pacilitaxel and/or thoracic radiotherapy to patients with non-small cell lung cancer.

In one aspect, the methods provided herein comprise administering a formulation of Compound 1 with taxotere to patients with stage IIIB or IV non-small cell lung cancer.

In one aspect, the methods provided herein comprise administering a formulation of Compound 1 with oblimersen (Genasense®), methotrexate, mechlorethamine hydrochloride, etoposide, topotecan and/or doxorubicin to patients with small cell lung cancer.

In one aspect, the methods provided herein comprise administering a formulation of Compound 1 with Venetoclax, ABT-737 (Abbott Laboratories) and/or obatoclax (GX15-070) to patients with lymphoma and other blood cancers.

In one aspect, the methods provided herein comprise administering a formulation of Compound 1 with a second active ingredient such as vinblastine or fludarabine adcetris, ambochlorin, becenum, bleomycin, brentuximab vedotin, carmustinem chlorambucil, cyclophosphamide, dacarbazine, doxorubicin, lomustine, matulane, mechlorethamine hydrochloride, prednisone, procarbazine hydrochloride, vincristine, methotrexate, nelarabin, belinostat, bendamustine HCl, tositumomab, and iodine 131 tositumomab, denileukin diftitox, dexamethasone, pralatrexate, prelixafor, obinutuzumab, ibritumomab, tiuxefan, ibritinib, idelasib, intron A, romidepsin, lenalidomide, rituximab, and/or vorinostat to patients with various types of lymphoma, including, but not limited to, Hodgkin's lymphoma, non-Hodgkin's lymphoma, cutaneous T-Cell lymphoma, cutaneous B-Cell lymphoma, diffuse large B-Cell lymphoma or relapsed or refractory low grade follicular lymphoma.

In one aspect, the methods provided herein comprise administering a formulation of Compound 1 with taxotere, dabrafenib, imlygic, ipilimumab, pembrolizumab, nivolumab, trametinib, vemurafenib, talimogene laherparepvec, IL-2, IFN, GM-CSF, and/or dacarbazine, aldesleukin, cobimetinib, Intron A®, peginterferon Alfa-2b, and/or trametinib to patients with various types or stages of melanoma.

In one aspect, the methods provided herein comprise administering a formulation of Compound 1 with vinorelbine or pemetrexed disodium to patients with malignant mesothelioma, or stage IIIB non-small cell lung cancer with pleural implants or malignant pleural effusion mesothelioma syndrome.

In one aspect, the methods of treating patients with various types or stages of multiple myeloma provided herein comprise administering a formulation of Compound 1 with with dexamethasone, zoledronic acid, palmitronate, GM-CSF, biaxin, vinblastine, melphalan, busulphan, cyclophosphamide, IFN, prednisone, bisphosphonate, celecoxib, arsenic trioxide, PEG INTRON-A, vincristine, becenum, bortezomib, carfilzomib, doxorubicin, panobinostat, lenalidomide, pomalidomide, thalidomide, mozobil, carmustine, daratumumab, elotuzumab, ixazomib citrate, plerixafor or a combination thereof.

In certain embodiments, a formulation of Compound 1 provided herein is administered to patients with various types or stages of multiple myeloma in combination with chimeric antigen receptor (CAR) T-cells. In certain embodiments the CAR T cell in the combination targets B cell maturation antigen (BCMA), and in more specific embodiments, the CAR T cell is bb2121 or bb21217. In some embodiments, the CAR T cell is JCARH125.

In certain embodiments, a formulation of Compound 1 provided herein is administered to patients with relapsed or refractory multiple myeloma in combination with doxorubicin (Doxil®), vincristine and/or dexamethasone (Decadron®).

In certain embodiments, the methods provided herein comprise administering a formulation of Compound 1 to patients with various types or stages of ovarian cancer such as peritoneal carcinoma, papillary serous carcinoma, refractory ovarian cancer or recurrent ovarian cancer, in combination with taxol, carboplatin, doxorubicin, gemcitabine, cisplatin, xeloda, paclitaxel, dexamethasone, avastin, cyclophosphamide, topotecan, olaparib, thiotepa, melphalan, niraparib tosylate monohydrate, rubraca or a combination thereof.

In certain embodiments, the methods provided herein comprise administering a formulation of Compound 1 to patients with various types or stages of prostate cancer, in combination with xeloda, 5 FU/LV, gemcitabine, irinotecan plus gemcitabine, cyclophosphamide, vincristine, dexamethasone, GM-CSF, celecoxib, taxotere, ganciclovir, paclitaxel, adriamycin, docetaxel, estramustine, Emcyt, denderon, zytiga, bicalutamide, cabazitaxel, degarelix, enzalutamide, zoladex, leuprolide acetate, mitoxantrone hydrochloride, prednisone, sipuleucel-T, radium 223 dichloride, or a combination thereof.

In certain embodiments, the methods provided herein comprise administering a formulation of Compound 1 to patients with various types or stages of renal cell cancer, in combination with capecitabine, IFN, tamoxifen, IL-2, GM-CSF, Celebrex®, flutamide, goserelin acetate, nilutamide or a combination thereof.

In certain embodiments, the methods provided herein comprise administering a formulation of Compound 1 to patients with various types or stages of gynecologic, uterus or soft tissue sarcoma cancer in combination with IFN, dactinomycin, doxorubicin, imatinib mesylate, pazopanib, hydrochloride, trabectedin, eribulin mesylate, olaratumab, a COX-2 inhibitor such as celecoxib, and/or sulindac.

In one aspect, the methods provided herein comprise administering a formulation of Compound 1 to patients with various types or stages of solid tumors in combination with celecoxib, etoposide, cyclophosphamide, docetaxel, apecitabine, IFN, tamoxifen, IL-2, GM-CSF, or a combination thereof.

In one aspect, the methods provided herein comprise administering a formulation of Compound 1 to patients with scleroderma or cutaneous vasculitis in combination with celebrex, etoposide, cyclophosphamide, docetaxel, apecitabine, IFN, tamoxifen, IL-2, GM-CSF, or a combination thereof.

In one aspect, the methods provided herein comprise administering a formulation of Compound 1 to patients with MDS in combination with azacitidine, cytarabine, daunorubicin, decitabine, idarubicin, lenalidomide, enasidenib, or a combination thereof.

In one aspect, the methods provided herein comprise administering a formulation of Compound 1 to patients with a hematological cancer in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In certain embodiments, a formulation of Compound 1 provided herein is administered to patients with leukemia in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In certain embodiments, a formulation of Compound 1 provided herein is administered to patients with AML in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering a formulation of Compound 1 to patients with leukemia in combination with an mTOR inhibitor. In certain embodiments, a formulation of Compound 1 provided herein is administered to patients with leukemia in combination with an mTOR inhibitor. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, Compound 1 is administered to patients with leukemia in combination with 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223). In certain embodiments, a formulation of Compound 1 is administered to patients with leukemia in combination with 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, a formulation of Compound 1 is administered to patients with leukemia in combination with everolimus. In certain embodiments, a formulation of Compound 1 is administered to patients with leukemia in combination with MLN-0128. In certain embodiments, a formulation of Compound 1 is administered to patients with leukemia in combination with AZD8055.

In certain embodiments, a formulation of Compound 1 provided herein is administered to patients with AML in combination with an mTOR inhibitor. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, a formulation of Compound 1 is administered to patients with AML in combination with 1-ethyl-7-(2-methyl-6-(1H-1,2,4-triazol-3-yl)pyridin-3-yl)-3,4-dihydropyrazino[2,3-b]pyrazin-2(1H)-one. In certain embodiments, a formulation of Compound 1 is administered to patients with AML in combination with everolimus. In certain embodiments, everolimus is administered to patients with AML prior to administration of a formulation of Compound 1. In certain embodiments, a formulation of Compound 1 is administered to patients with AML in combination with MLN-0128. In certain embodiments, a formulation of Compound 1 is administered to patients with AML in combination with AZD8055.

In certain embodiments, a formulation of Compound 1 provided herein is administered to patients with MPN in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from tofacitinib, momelotinib, ruxolitinib, fedratinib, NS-018 and pacritinib. In certain embodiments, a formulation of Compound 1 is administered to patients with MPN in combination with tofacitinib. In certain embodiments, a formulation of Compound 1 is administered to patients with MPN in combination with momelotinib. In certain embodiments, a formulation of Compound 1 is administered to patients with MPN in combination with filgotinib. In certain embodiments, a formulation of Compound 1 is administered to patients with MPN in combination with decernotinib. In certain embodiments, a formulation of Compound 1 is administered to patients with MPN in combination with barcitinib. In certain embodiments, a formulation of Compound 1 is administered to patients with MPN in combination with ruxolitinib. In certain embodiments, a formulation of Compound 1 is administered to patients with MPN in combination with fedratinib. In certain embodiments, a formulation of Compound 1 is administered to patients with MPN in combination with NS-018. In certain embodiments, a formulation of Compound 1 is administered to patients with MPN in combination with pacritinib. In certain embodiments, the MPN is IL-3 independent. In certain embodiments, the MPN is characterized by a JAK 2 mutation, for example, a JAK2^{V617F} mutation.

In certain embodiments, a formulation of Compound 1 provided herein is administered to patients with myelofibrosis in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, ruxolitinib, fedratinib, NS-018 and pacritinib. In certain embodiments, a formulation of Compound 1 is administered to patients with myelofibrosis in combination with tofacitinib. In certain embodiments, a formulation of Compound 1 is administered to patients with myelofibrosis in combination with momelotinib. In certain embodiments, a formulation of Compound 1 is administered to patients with myelofibrosis in combination with ruxolitinib. In certain embodiments, a formulation of Compound 1 is administered to patients with myelofibrosis in combination with fedratinib. In certain embodiments, a formulation of Compound 1 is administered to patients with myelofibrosis in combination with NS-018. In certain embodiments, a formulation of Compound 1 is administered to patients with myelofibrosis in combination with pacritinib. In certain embodiments, the myeolofibrosis is characterized by a JAK 2 mutation, for example, a JAK2V617F mutation. In some embodiments, the myelofibrosis is primary myelofibrosis. In other embodiments, the myelofibrosis is secondary myelofibrosis. In some such embodiments, the secondary myelofibrosis is post polycythemia vera myelofibrosis. In other embodiments, the secondary myelofibrosis is post essential thrombocythemia myelofibrosis.

In certain embodiments, a formulation of Compound 1 provided herein is administered to patients with leukemia in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from momelotinib, ruxolitinib, fedratinib, NS-018 and pacritinib. In certain embodiments, a formulation of Compound 1 is administered to patients with leukemia in combination with tofacitinib. In certain embodiments, a formulation of Compound 1 is administered to patients with leukemia in combination with momelotinib. In certain embodiments, a formulation of Compound 1 is administered to patients with leukemia in combination with filgotinib. In certain embodiments, a formulation of Compound 1 is administered to patients with leukemia in combination with decernotinib. In certain embodiments, a formulation of Compound 1 is administered to patients with leukemia in combination with barcitinib. In certain embodiments, a formulation of Compound 1 is administered to patients with leukemia in combination with ruxolitinib. In certain embodiments, a formulation of Compound 1 is administered to patients with leukemia in combination with fedratinib. In certain embodiments, a formulation of Compound 1 is administered to patients with leukemia in combination with NS-018. In certain embodiments, a formulation of Compound 1 is administered to patients with leukemia in combination with pacritinib. In certain embodiments, the MPN is characterized by a JAK 2 mutation, for example, a JAK2V617F mutation.

In certain embodiments, a formulation of Compound 1 provided herein is administered to patients with AML in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from momelotinib, ruxolitinib, fedratinib, NS-018 and pacritinib. In certain embodiments, a formulation of Compound 1 is administered to patients with AML in combination with tofacitinib. In certain embodiments, a formulation of Compound 1 is administered to patients with AML in combination with momelotinib. In certain embodiments, a formulation of Compound 1 is administered to patients with AML in combination with filgotinib. In certain embodiments, a formulation of Compound 1 is administered to patients with AML in combination with decernotinib. In certain embodiments, a formulation of Compound 1 is administered to patients with AML in combination with barcitinib. In certain embodiments, a formulation of Compound 1 is administered to patients with AML in combination with ruxolitinib. In certain embodiments, a formulation of Compound 1 is administered to patients with AML in combination with fedratinib. In certain embodiments, a formulation of Compound 1 is administered to patients with AML in combination with NS-018. In certain embodiments, a formulation of Compound 1 is administered to patients with AML in combination with pacritinib. In certain embodiments, the MPN is characterized by a JAK 2 mutation, for example, a JAK2V617F mutation.

In certain embodiments, a formulation of Compound 1 provided herein is administered to patients with leukemia in combination with a FLT3 kinase inhibitor. In certain embodiments, the FLT3 kinase inhibitor is selected from quizartinib, sunitinib, sunitinib malate, midostaurin, pexidartinib, lestaurtinib, tandutinib, and crenolanib. In certain embodiments, a formulation of Compound 1 is administered to patients with leukemia in combination with quizartinib. In certain embodiments, a formulation of Compound 1 is administered to patients with leukemia in combination with sunitinib. In certain embodiments, Compound 1 is administered to patients with leukemia in combination with midostaurin. In certain embodiments, a formulation of Compound 1 is administered to patients with leukemia in combination with pexidartinib. In certain embodiments, a formulation of Compound 1 is administered to patients with leukemia in combination with lestaurtinib. In certain embodiments, a formulation of Compound 1 is administered to patients with leukemia in combination with tandutinib. In certain embodiments, a formulation of Compound 1 is administered to patients with leukemia in combination with crenolanib. In certain embodiments, the patient carries a FLT3-ITD mutation.

In certain embodiments, a formulation of Compound 1 provided herein is administered to patients with AML in combination with a FLT3 kinase inhibitor. In certain embodiments, the FLT3 kinase inhibitor is selected from quizartinib, sunitinib, sunitinib malate, midostaurin, pexidartinib, lestaurtinib, tandutinib, quizartinib and crenolanib. In certain embodiments, a formulation of Compound 1 is administered to patients with AML in combination with quizartinib. In certain embodiments, a formulation of Compound 1 is administered to patients with AML in combination with sunitinib. In certain embodiments, a formulation of Compound 1 is administered to patients with AML in combination with midostaurin. In certain embodiments, a formulation of Compound 1 is administered to patients with AML in combination with pexidartinib. In certain embodiments, a formulation of Compound 1 is administered to patients with AML in combination with lestaurtinib. In certain embodiments, a formulation of Compound 1 is administered to patients with AML in combination with tandutinib. In certain embodiments, a formulation of Compound 1 is administered to patients with AML in combination with crenolanib. In certain embodiments, the patient carries a FLT3-ITD mutation.

In certain embodiments, a formulation of Compound 1 is administered to patients with leukemia in combination with a spliceosome inhibitor. In certain embodiments, a formulation of Compound 1 is administered to patients with AML in combination with a spliceosome inhibitor. In certain embodiments, the spliceosome inhibitor is pladienolide B, 6-deoxypladienolide D, or H3B-8800.

In certain embodiments, a formulation of Compound 1 provided herein is administered to patients with leukemia in combination with an SMG1 kinase inhibitor. In certain embodiments, a formulation of Compound 1 provided herein is administered to patients with AML in combination with an SMG1 kinase inhibitor. In certain embodiments, the SMG1 inhibitor is 1-ethyl-7-(2-methyl-6-(1H-1,2,4-triazol-3-yl)pyridin-3-yl)-3,4-dihydropyrazino[2,3-b]pyrazin-2(1H)-one, chloro-N,N-diethyl-5-((4-(2-(4-(3-methylureido)phenyl)pyridin-4-yl)pyrimidin-2-yl)amino)benzenesulfonamide (compound Ii), or a compound disclosed in A. Gopalsamy et al, Bioorg. Med Chem Lett. 2012, 22:6636-66412 (for example, chloro-N,N-diethyl-5-((4-(2-(4-(3-methylureido)phenyl)pyridin-4-yl)pyrimidin-2-yl)amino)benzenesulfonamide.

In certain embodiments, a formulation of Compound 1 provided herein is administered to patients with leukemia in combination with a BCL2 inhibitor. In certain embodiments, a formulation of Compound 1 provided herein is administered to patients with AML in combination with a BCL2 inhibitor, for example, venetoclax or navitoclax. In certain embodiments, the BCL2 inhibitor is venetoclax.

In one embodiment, provided herein is a method for treating of AML that is resistant to treatment with a BCL2 inhibitor, comprising administering a formulation of Compound 1. In one embodiment, provided herein is a method for treating of AML that has acquired resistance to venetoclax treatment, comprising administering Compound 1. In one embodiment, provided herein is a method for treating of AML that has acquired resistance to venetoclax treatment, comprising administering a combination of a formulation of Compound 1 and a BCL2 inhibitor. In one embodiment, provided herein is a method for treating of AML that has acquired resistance to venetoclax treatment, comprising administering a combination of a formulation of Compound 1 and venetoclax.

In certain embodiments, a formulation of Compound 1 provided herein is administered to patients with leukemia in combination with a topoisomerase inhibitor. In certain embodiments, a formulation of Compound 1 provided herein is administered to patients with AML in combination with a topoisomerase inhibitor, for example, irinotecan, topotecan, camptothecin, lamellarin D, etoposide, teniposide, doxorubicin, daunorubicin, mitoxantrone, amsacrine, ellipticines, aurintricarboxylic acid, or HU-331. In certain embodiments, the topoisomerase inhibitor is topotecan.

In certain embodiments, a formulation of Compound 1 is administered to patients with leukemia in combination with a BET inhibitor. In certain embodiments, a formulation of Compound 1 is administered to patients with AML in combination with a BET inhibitor. In certain embodiments, the BET inhibitor is selected from GSK525762A, OTX015, BMS-986158, TEN-010, CPI-0610, INCB54329, BAY1238097, FT-1101, C90010, ABBV-075, BI 894999, GS-5829, GSK1210151A (I-BET-151), CPI-203, RVX 208, XD46, MS436, PFI-1, RVX2135, ZEN3365, XD14, ARV-771, MZ-1, PLX5117, 4-[2-(cyclopropylmethoxy)-5-(methanesulfonyl)phenyl]-2-methylisoquinolin-1(2H)-one (Compound A), EP11313 and EP11336.

In certain embodiments, a formulation of Compound 1 is administered to patients with leukemia in combination with an LSD1 inhibitor. In certain embodiments, a formulation of Compound 1 is administered to patients with AML in combination with an LSD1 inhibitor. In certain embodiments, the LSD1 inhibitor is selected from ORY-1001, ORY-2001, INCB-59872, IMG-7289, TAK 418, GSK-2879552, and 4-[2-(4-amino-piperidin-1-yl)-5-(3-fluoro-4-methoxy-phenyl)-1-methyl-6-oxo-1,6-dihydropyrimidin-4-yl]-2-fluoro-benzonitrile or a salt thereof (e.g. besylate salt, Compound B).

In one aspect, the methods provided herein comprise administering a formulation of Compound 1 to patients with leukemia in combination with triptolide, retaspimycin, alvespimycin, 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223), 1-ethyl-7-(2-methyl-6-(1H-1,2,4-triazol-3-yl)pyridin-3-yl)-3,4-dihydropyrazino[2,3-b]pyrazin-2(1H)-one (CC-115), rapamycin, MLN-0128, everolimus, AZD8055, pladienolide B, topotecan, thioguanine, mitoxantrone, etoposide, decitabine, daunorubicin, clofarabine, cladribine, 6-mercaptopurine, chloro-N,N-diethyl-5-((4-(2-(4-(3-methylureido)phenyl)pyridin-4-yl)pyrimidin-2-yl)amino)benzenesulfonamide (compound Ii), fedratinib, sunitinib, pexidartinib, midostaurin, lestaurtinib, momelotinib, quizartinib, and crenolanib.

In one aspect, the methods provided herein comprise administering a formulation of Compound 1 to patients with AML in combination with triptolide, retaspimycin, alvespimycin, 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223), 1-ethyl-7-(2-methyl-6-(1H-1,2,4-triazol-3-yl)pyridin-3-yl)-3,4-dihydropyrazino[2,3-b]pyrazin-2(1H)-one (CC-115), rapamycin, MLN-0128, everolimus, AZD8055, pladienolide B, topotecan, thioguanine, mitoxantrone, etoposide, decitabine, daunorubicin, clofarabine, cladribine, 6-mercaptopurine, chloro-N,N-diethyl-5-((4-(2-(4-(3-methylureido)phenyl)pyridin-4-yl)pyrimidin-2-yl)amino)benzenesulfonamide (compound Ii), fedratinib, sunitinib, pexidartinib, midostaurin, lestaurtinib, momelotinib, quizartinib, and crenolanib.

In certain embodiments, a formulation of Compound 1 provided herein is administered to patients with cancer in combination with a topoisomerase inhibitor. In certain embodiments, a formulation of Compound 1 provided herein is administered to cancer patients in combination with an mTOR inhibitor, wherein the cancer is selected from breast cancer, kidney cancer, pancreatic cancer, gastrointestinal cancer, lung cancer, neuroendocrine tumor (NET), and renal cell carcinoma. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In one embodiment, the mTOR kinase inhibitor is 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223). In one embodiment, the mTOR kinase inhibitor is 1-ethyl-7-(2-methyl-6-(1H-1,2,4-triazol-3-yl)pyridin-3-yl)-3,4-dihydropyrazino[2,3-b]pyrazin-2(1H)-one (CC-115). In one embodiment, the mTOR inhibitor is everolimus. In one embodiment, the mTOR inhibitor is temsirolimus. In one embodiment, the mTOR inhibitor is MLN-0128. In one embodiment, the mTOR inhibitor is AZD8055.

In certain embodiments, a formulation of Compound 1 provided herein is administered to breast cancer patients in combination with everolimus.

In certain embodiments, a formulation of Compound 1 provided herein is administered to kidney cancer patients in combination with everolimus.

In certain embodiments, a formulation of Compound 1 provided herein is administered to pancreatic cancer patients in combination with everolimus.

In certain embodiments, a formulation of Compound 1 provided herein is administered to gastrointestinal cancer patients in combination with everolimus.

In certain embodiments, a formulation of Compound 1 provided herein is administered to lung cancer patients in combination with everolimus.

In certain embodiments, a formulation of Compound 1 provided herein is administered to neuroendocrine tumor patients in combination with everolimus.

In certain embodiments, a formulation of Compound 1 provided herein is administered to renal cell carcinoma patients in combination with everolimus.

Also encompassed herein is a method of increasing the dosage of an anti-cancer drug or agent that can be safely and effectively administered to a patient, which comprises administering to the patient (e.g., a human) a formulation of Compound 1 provided herein in combination with the second anti-cancer drug. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of a formulation of Compound 1 provided herein, alleviates or reduces adverse effects which are of such severity that it would otherwise limit the amount of anti-cancer drug.

Also encompassed herein is a method of decreasing the dosage of an anti-cancer drug or agent that can be safely and effectively administered to a patient, which comprises administering to the patient (e.g., a human) a formulation of Compound 1 provided herein in combination with the second anti-cancer drug. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of a formulation of Compound 1 provided herein, potentiates the activity of the anti-cancer drug, which allows for a reduction in dose of the anti-cancer drug while maintaining efficacy, which in turn can alleviate or reduce the adverse effects which are of such severity that it limited the amount of anti-cancer drug.

In one embodiment, a formulation of Compound 1 provided herein is administered daily in an amount ranging from about 0.1 to about 20 mg, from about 1 to about 15 mg, from about 1 to about 10 mg, or from about 1 to about 15 mg prior to, during, or after the occurrence of the adverse effect associated with the administration of an anti-cancer drug to a patient. In certain embodiments, a formulation of Compound 1 provided herein is administered in combination with specific agents such as heparin, aspirin, coumadin, or G-CSF to avoid adverse effects that are associated with anti-cancer drugs such as but not limited to neutropenia or thrombocytopenia.

In one embodiment, a formulation of Compound 1 provided herein, is administered to patients with diseases and disorders associated with or characterized by, undesired angiogenesis in combination with additional active ingredients, including, but not limited to, anti-cancer drugs, anti-inflammatories, antihistamines, antibiotics, and steroids.

In another embodiment, encompassed herein is a method of treating, preventing and/or managing cancer, which comprises administering a formulation of Compound 1 provided herein, in conjunction with (e.g. before, during, or after) at least one anti-cancer therapy including, but not limited to, surgery, immunotherapy, biological therapy, radiation therapy, or other non-drug based therapy presently used to treat, prevent and/or manage cancer. The combined use of the compound provided herein and other anti-cancer therapy may provide a unique treatment regimen that is unexpectedly effective in certain patients. Without being limited by theory, it is believed that Compound 1 may provide additive or synergistic effects when given concurrently with at least one anti-cancer therapy.

As discussed elsewhere herein, encompassed herein is a method of reducing, treating and/or preventing adverse or undesired effects associated with other anti-cancer therapy including, but not limited to, surgery, chemotherapy, radiation therapy, hormonal therapy, biological therapy and immunotherapy. A formulation of Compound 1 provided herein, and other active ingredient can be administered to a patient prior to, during, or after the occurrence of the adverse effect associated with other anti-cancer therapy.

In certain embodiments, the methods provided herein comprise administration of one or more of calcium, calcitriol, or vitamin D supplementation with a formulation of Compound 1. In certain embodiments, the methods provided herein comprise administration of calcium, calcitriol, and vitamin D supplementation prior to the treatment with a formulation of Compound 1. In certain embodiments, the methods provided herein comprise administration of calcium, calcitriol, and vitamin D supplementation prior to the administration of first dose of a formulation of Compound 1 in each cycle. In certain embodiments, the methods provided herein comprise administration of calcium, calcitriol, and vitamin D supplementation at least up to 3 days prior to the treatment with a formulation of Compound 1. In certain embodiments, the methods provided herein comprise administration of calcium, calcitriol, and vitamin D supplementation prior to the administration of first dose of a formulation of Compound 1 in each cycle. In certain embodiments, the methods provided herein comprise administration of calcium, calcitriol, and vitamin D supplementation at least up to 3 days prior to the administration of first dose of a formulation of Compound 1 in each cycle. In certain embodiments, the methods provided herein comprise administration of calcium, calcitriol, and vitamin D supplementation prior to administration of first dose of a formulation of Compound 1 in each cycle and continues after administration of the last dose of a formulation of Compound 1 in each cycle. In certain embodiments, the methods provided herein comprise administration of calcium, calcitriol, and vitamin D supplementation at least up to 3 days prior to administration of first dose of a formulation of Compound 1 in each cycle and continues until at least up to 3 days after administration of the last dose of a formulation of Compound 1 in each cycle (e.g., at least up to day 8 when Compound 1 is administered on Days 1-5). In one embodiment, the methods provided herein comprise administration of calcium, calcitriol, and vitamin D supplementation at least up to 3 days prior to administration of day 1 of each cycle and continue until >3 days after the last dose of a formulation of Compound 1 in each cycle (eg, >Day 8 when Compound 1 is administered on Days 1-5, >Day 13 when Compound 1 is administered on Days 1-3 and Days 8-10).

In certain embodiments, calcium supplementation is administered to deliver at least 1200 mg of elemental calcium per day given in divided doses. In certain embodiments, calcium supplementation is administered as calcium carbonate in a dose of 500 mg administered three times a day per orally (PO).

In certain embodiments, calcitriol supplementation is administered to deliver 0.25 μg calcitriol (PO) once daily.

In certain embodiments, vitamin D supplementation is administered to deliver about 500 IU to about 50,000 IU vitamin D once daily. In certain embodiments, vitamin D supplementation is administered to deliver about 1000 IU vitamin D once daily. In certain embodiments, vitamin D supplementation is administered to deliver about 50,000 IU vitamin D weekly. In certain embodiments, vitamin D supplementation is administered to deliver about 1000 IU vitamin D2 or D3 once daily. In certain embodiments, vitamin D supplementation is administered to deliver about 500 IU vitamin D once daily. In certain embodiments, vitamin D supplementation is administered to deliver about 50,000 IU vitamin D weekly. In certain embodiments, vitamin D supplementation is administered to deliver about 20,000 IU vitamin D weekly. In certain embodiments, vitamin D supplementation is administered to deliver about 1000 IU vitamin D2 or D3 once daily. In certain embodiments, vitamin D supplementation is administered to deliver about 50,000 IU vitamin D2 or D3 weekly. In certain embodiments, vitamin D supplementation is administered to deliver about 20,000 IU vitamin D2 or D3 weekly.

In certain embodiments, a formulation of Compound 1 provided herein and doxetaxol are administered to patients with non-small cell lung cancer who were previously treated with carbo/VP 16 and radiotherapy.

Use with Transplantation Therapy

A formulation of Compound 1 provided herein, can be used to reduce the risk of Graft Versus Host Disease (GVHD). Therefore, encompassed herein is a method of treating, preventing and/or managing cancer, which comprises administering a formulation of Compound 1 provided herein, in conjunction with transplantation therapy.

As those of ordinary skill in the art are aware, the treatment of cancer is often based on the stages and mechanism of the disease. For example, as inevitable leukemic transformation develops in certain stages of cancer, transplantation of peripheral blood stem cells, hematopoietic stem cell preparation or bone marrow may be necessary. The combined use of a formulation of Compound 1 provided herein, and transplantation therapy provides a unique and unexpected synergism. In particular, a formulation of Compound 1 provided herein exhibits immunomodulatory activity that may provide additive or synergistic effects when given concurrently with transplantation therapy in patients with cancer.

A formulation of Compound 1 provided herein, can work in combination with transplantation therapy reducing complications associated with the invasive procedure of transplantation and risk of GVHD. Encompassed herein is a method of treating, preventing and/or managing cancer which comprises administering to a patient (e.g., a human) formulation of Compound 1 provided herein before, during, or after the transplantation of umbilical cord blood, placental blood, peripheral blood stem cell, hematopoietic stem cell preparation, or bone marrow. Some examples of stem cells suitable for use in the methods provided herein are disclosed in U.S. Pat. No. 7,498,171, the disclosure of which is incorporated herein by reference in its entirety.

In one embodiment, a formulation of Compound 1 provided herein, is administered to patients with acute myeloid leukemia before, during, or after transplantation.

In one embodiment, a formulation of Compound 1 provided herein, is administered to patients with multiple myeloma before, during, or after the transplantation of autologous peripheral blood progenitor cells.

In one embodiment, a formulation of Compound 1 provided herein, is administered to patients with NHL (e.g., DLBCL) before, during, or after the transplantation of autologous peripheral blood progenitor cells.

Cycling Therapy

In certain embodiments, a formulation of Compound 1 provided herein, are cyclically administered to a patient independent of the cancer treated. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improve the efficacy of the treatment.

In certain embodiments, a formulation of Compound 1 provided herein, is administered daily in a single or divided dose in a four to six week cycle with a rest period of about a week or two weeks. In certain embodiments, a formulation of Compound 1 provided herein, is administered daily in a single or divided doses for one to ten consecutive days of a 28 day cycle, then a rest period with no administration for rest of the 28 day cycle. The cycling method further allows the frequency, number, and length of dosing cycles to be increased. Thus, encompassed herein in certain embodiments is the administration of a formulation of Compound 1 provided herein, for more cycles than are typical when it is administered alone. In certain embodiments, a formulation of Compound 1 provided herein, is administered for a greater number of cycles that would typically cause dose-limiting toxicity in a patient to whom a second active ingredient is not also being administered.

In one embodiment, a formulation of Compound 1 provided herein, is administered daily and continuously for three or four weeks to administer a dose of Compound 1 from about 0.1 to about 20 mg/d followed by a break of one or two weeks.

In another embodiment, a formulation of Compound 1 provided herein, is administered intravenously and a second active ingredient is administered orally, with administration of a formulation of Compound 1 provided herein, occurring 30 to 60 minutes prior to a second active ingredient, during a cycle of four to six weeks. In certain embodiments, the combination of a formulation of Compound 1 provided herein, and a second active ingredient is administered by intravenous infusion over about 90 minutes every cycle. In certain embodiments, one cycle comprises the administration from about 0.1 to about 150 mg/day of a formulation of Compound 1 provided herein, and from about 50 to about 200 mg/m²/day of a second active ingredient daily for three to four weeks and then one or two weeks of rest. In certain embodiments, the number of cycles during which the combinatorial treatment is administered to a patient is ranging from about one to about 24 cycles, from about two to about 16 cycles, or from about four to about three cycles.

In one embodiment, a cycling therapy provided herein comprises administering a formulation of Compound 1 in a treatment cycle which includes an administration period of up to 3 days followed by a rest period. In one embodiment, the treatment cycle includes an administration period of 3 days followed by a rest period. In one embodiment, a cycling therapy provided herein comprises administering a formulation of Compound 1 provided herein, in a treatment cycle which includes an administration period of up to 5 days followed by a rest period. In one embodiment, the treatment cycle includes an administration period of 5 days followed by a rest period. In one embodiment, a cycling therapy provided herein comprises administering a formulation of Compound 1 in a treatment cycle which includes an administration period of up to 7 days followed by a rest period. In one embodiment, the treatment cycle includes an administration period of 7 days followed by a rest period. In one embodiment, the treatment cycle includes an administration period of up to 10 days followed by a rest period. In one embodiment, the rest period is from about 10 days up to about 40 days. In one embodiment, the treatment cycle includes an administration period of up to 10 days followed by a rest period from about 10 days up to about 40 days. In one embodiment, the treatment cycle includes an administration period of up to 10 days followed by a rest period from about 23 days up to about 37 days. In one embodiment, the rest period is from about 23 days up to about 37 days. In one embodiment, the rest period is 23 days. In one embodiment, the treatment cycle includes an administration period of up to 10 days followed by a rest period of 23 days. In one embodiment, the rest period is 37 days. In one embodiment, the treatment cycle includes an administration period of up to 10 days followed by a rest period of 37 days.

In one embodiment, the treatment cycle includes an administration of a formulation of Compound 1 provided herein, on days 1 to 3 of a 28 day cycle. In one embodiment, the treatment cycle includes an administration of a formulation of Compound 1 provided herein, on days 1 to 5 of a 28 day cycle. In one embodiment, the treatment cycle includes an administration of a formulation of Compound 1 provided herein, on days 1 to 7 of a 28 day cycle. In another embodiment, the treatment cycle includes an administration of a formulation of Compound 1 provided herein, on days 1-10 of a 28 day cycle. In one embodiment, the treatment cycle includes an administration on days 1 to 5 of a 42 day cycle. In another embodiment, the treatment cycle includes an administration on days 1-10 of a 42 day cycle. In another embodiment, the treatment cycle includes an administration on days 1-5 and 15-19 of a 28 day cycle. In another embodiment, the treatment cycle includes an administration on days 1-3 and 8-10 of a 28 day cycle.

In one embodiment, the treatment cycle includes an administration of a formulation of Compound 1 provided herein, on days 1 to 21 of a 28 day cycle. In another embodiment, the treatment cycle includes an administration on days 1 to 5 of a 7 day cycle. In another embodiment, the treatment cycle includes an administration on days 1 to 7 of a 7 day cycle. In one embodiment, the treatment cycle includes an administration of a formulation of Compound 1 on days 1 to 5 of a 21 day cycle. In one embodiment, the treatment cycle includes an administration of a formulation of Compound 1 on days 1 to 7 of a 21 day cycle. In one embodiment, the treatment cycle includes an administration of a formulation of Compound 1 on days 1 to 7 of a 28 day cycle.

Any treatment cycle described herein can be repeated for at least 2, 3, 4, 5, 6, 7, 8, or more cycles. In certain instances, the treatment cycle as described herein includes from 1 to about 24 cycles, from about 2 to about 16 cycles, or from about 2 to about 4 cycles. In certain instances a treatment cycle as described herein includes from 1 to about 4 cycles. In certain embodiments, cycle 1 to 4 are all 28 day cycles. In certain embodiments, cycle 1 is a 42 day cycle and cycles 2 to 4 are 28 day cycles. In some embodiments, Compound 1, for example, a formulation of Compound 1 provided herein, is administered for 1 to 13 cycles of 28 days (e.g. about 1 year). In certain instances, the cycling therapy is not limited to the number of cycles, and the therapy is continued until disease progression. Cycles, can in certain instances, include varying the duration of administration periods and/or rest periods described herein.

In one embodiment the treatment cycle includes administering Compound 1 at a dosage amount of about 0.3 mg/day, 0.6 mg/day, 1.2 mg/day, 1.8 mg/day, 2.4 mg/day, 3.6 mg/day, 4.5 mg/day, 5.4 mg/day, 7.2 mg/day, 8.1 mg/day, 9.0 mg/day, 10.0 mg/day, 10.8 mg/day, or 12.2 mg/day administered once per day.

In one embodiment the treatment cycle includes administering Compound 1 at a dosage amount of about 0.3 mg/day, 0.6 mg/day, 1.2 mg/day, 1.8 mg/day, 2.4 mg/day, 3.6 mg/day, 5.4 mg/day, 7.2 mg/day, 8.1 mg/day, 9.0 mg/day, 10.0 mg/day, 10.8 mg/day, or 12.2 mg/day administered once per day. In one embodiment the treatment cycle includes administering Compound 1 at a dosage amount of about 0.3 mg/day, 0.6 mg/day, 1.2 mg/day, 1.8 mg/day, 2.4 mg/day, 3.6 mg/day, 5.4 mg/day, 7.2 mg/day, 8.1 mg/day, 9.0 mg/day, 10.0 mg/day, 10.8 mg/day, 12.2 mg/day, or 20 mg/day administered once per day. In one embodiment the treatment cycle includes administering Compound 1 at a dosage amount of about 0.6 mg/day, 1.2 mg/day, 1.8 mg/day, 2.4 mg/day, or 3.6 mg/day, administered once per day. In some such embodiments, the treatment cycle includes administering Compound 1 at a dosage amount of about 0.6 mg, 1.2 mg, 1.8 mg, 2.4 mg, 3.6 mg or 4.5 mg on days 1 to 3 of a 28 day cycle. In some such embodiments, the treatment cycle includes administering Compound 1 at a dosage amount of about 0.6 mg, 1.2 mg, 1.8 mg, 2.4 mg, or 3.6 mg on days 1 to 3 of a 28 day cycle. In other embodiments, the treatment cycle includes administering a formulation of Compound 1 at a dosage amount of about 0.6 mg, 1.2 mg, 1.8 mg, 2.4 mg, 3.6 mg or 4.5 mg on days 1 to 5 and 15 to 19 of a 28 day cycle. In other embodiments, the treatment cycle includes administering a formulation of Compound 1 at a dosage amount of about 0.6 mg, 1.2 mg, 1.8 mg, 2.4 mg, or 3.6 mg on days 1 to 5 and 15 to 19 of a 28 day cycle. In other embodiments, the treatment cycle includes administering a formulation of Compound 1 at a dosage amount of about 0.6 mg, 1.2 mg, 1.8 mg, 2.4 mg, 3.6 mg, 5.4 mg/day, 7.2 mg/day, 8.1 mg/day, 9.0 mg/day, or 10.0 mg/day, on days 1 to 5 and 15 to 19 of a 28 day cycle. In other embodiments, the treatment cycle includes administering a formulation of Compound 1 at a dosage amount of about 0.6 mg, 1.2 mg, 1.8 mg, 2.4 mg, or 3.6 mg on days 1 to 5 of a 28 day cycle.

In some such embodiments, the treatment cycle includes administering a formulation of Compound 1 at a dosage amount of about 2.4 mg on days 1 to 5 of a 28 day cycle. In some such embodiments, the treatment cycle includes administering Compound 1 at a dosage amount of about 3.6 mg on days 1 to 5 of a 28 day cycle.

A formulation of Compound 1 provided herein, can be administered at the same amount for all administration periods in a treatment cycle. Alternatively, in one embodiment, the compound is administered at different doses in the administration periods.

In some embodiments, the treatment cycle includes administering Compound 1 at a first dosage amount on days 1 to 3, and at a second dosage amount on days 8 to 10 of a 28 day cycle, wherein the first and the second dosage amounts are the same or different. In some such embodiments, the treatment cycle includes administering Compound 1 at a dosage amount of about 2.4 mg on days 1 to 3, and at a dosage amount of about 3.6 mg on days 8 to 10 of a 28 day cycle.

In one embodiment, a formulation of Compound 1 provided herein is administered to a subject in a cycle, wherein the cycle comprises administering the formulation for at least 5 days in a 28 day cycle. In one embodiment, a formulation of Compound 1 provided herein is administered to a subject in a cycle, wherein the cycle comprises administering the formulation on days 1 to 5 of a 28 day cycle. In one embodiment, the formulation is administered to deliver Compound 1 in a dose of about 0.1 mg to about 20 mg on days 1 to 5 of a 28 day cycle. In one embodiment, the formulation is administered to deliver Compound 1 in a dose of about 0.5 mg to about 5 mg on days 1 to 5 of a 28 day cycle. In one embodiment, the formulation is administered to deliver Compound 1 in a dose of about 0.5 mg to about 10 mg on days 1 to 5 of a 28 day cycle. In one embodiment, a formulation of Compound 1 provided herein is administered to a subject in a cycle, wherein the cycle comprises administering the formulation on days 1 to 5 and 15 to 19 of a 28 day cycle. In one embodiment, the formulation is administered to deliver Compound 1 in a dose of about 0.1 mg to about 20 mg on days 1 to 5 and 15 to 19 of a 28 day cycle. In one embodiment, the formulation is administered to deliver Compound 1 in a dose of about 0.5 mg to about 5 mg on days 1 to 5 and 15 to 19 of a 28 day cycle. In one embodiment, the formulation is administered to deliver Compound 1 in a dose of about 0.5 mg to about 10 mg on days 1 to 5 and 15 to 19 of a 28 day cycle.

In one embodiment, provided herein is a method of treating of AML by administering to a subject a formulation of Compound 1 provided herein in a cycle, wherein the cycle comprises administering the formulation to deliver Compound 1 in a dose of about 0.1 mg to about 20 mg for at least 5 days in a 28 day cycle. In one embodiment, provided herein is a method of treating of AML by administering to a subject a formulation of Compound 1 provided herein in a cycle, wherein the cycle comprises administering the formulation to deliver Compound 1 in a dose of about 0.1 mg to about 20 mg on days 1 to 5 of a 28 day cycle. In one embodiment, provided herein is a method of treating of AML by administering to a subject a formulation of Compound 1 provided herein in a cycle, wherein the cycle comprises administering the formulation to deliver Compound 1 in a dose of about 0.1 mg to about 5 mg on days 1 to 5 of a 28 day cycle. In one embodiment, provided herein is a method of treating of AML by administering to a subject a formulation of Compound 1 provided herein in a cycle, wherein the cycle comprises administering the formulation to deliver Compound 1 in a dose of about 0.5 mg to about 5 mg on days 1 to 5 of a 28 day cycle. In another embodiment, provided herein is a method of treating of AML by administering to a subject a formulation of Compound 1 provided herein in a cycle, wherein the cycle comprises administering the formulation to deliver Compound 1 in a dose of about 0.1 mg to about 20 mg on days 1 to 5 and 15 to 19 of a 28 day cycle. In one embodiment, provided herein is a method of treating of AML by administering to a subject a formulation of Compound 1 provided herein in a cycle, wherein the cycle comprises administering the formulation to deliver Compound 1 in a dose of about 0.1 mg to about 5 mg on days 1 to 5 and 15 to 19 of a 28 day cycle. In one embodiment, provided herein is a method of treating of AML by administering to a subject a formulation of Compound 1 provided herein in a cycle, wherein the cycle comprises administering the formulation to deliver Compound 1 in a dose of about 0.5 mg to about 5 mg on days 1 to 5 and 15 to 19 of a 28 day cycle. In one embodiment, provided herein is a method of treating of AML by administering to a subject a formulation of Compound 1 provided herein in a cycle, wherein the cycle comprises administering the formulation to deliver Compound 1 in a dose of about 0.5 mg to about 5 mg on days 1 to 5 of a 28 day cycle.

In one embodiment, provided herein is a method of treating of MDS by administering to a subject a formulation of Compound 1 provided herein in a cycle, wherein the cycle comprises administering the formulation to deliver Compound 1 in a dose of about 0.1 mg to about 20 mg for at least 5 days in a 28 day cycle. In one embodiment, provided herein is a method of treating of MDS by administering to a subject a formulation of Compound 1 provided herein in a cycle, wherein the cycle comprises administering the formulation to deliver Compound 1 in a dose of about 0.1 mg to about 20 mg on days 1 to 5 of a 28 day cycle. In one embodiment, provided herein is a method of treating of MDS by administering to a subject a formulation of Compound 1 provided herein in a cycle, wherein the cycle comprises administering the formulation to deliver Compound 1 in a dose of about 0.1 mg to about 5 mg on days 1 to 5 of a 28 day cycle. In one embodiment, provided herein is a method of treating of MDS by administering to a subject a formulation of Compound 1 provided herein in a cycle, wherein the cycle comprises administering the formulation to deliver Compound 1 in a dose of about 0.5 mg to about 5 mg on days 1 to 5 of a 28 day cycle. In another embodiment, provided herein is a method of treating of MDS by administering to a subject a formulation of Compound 1 provided herein in a cycle, wherein the cycle comprises administering the formulation to deliver Compound 1 in a dose of about 0.1 mg to about 20 mg on days 1 to 5 and 15 to 19 of a 28 day cycle. In one embodiment, provided herein is a method of treating of MDS by administering to a subject a formulation of Compound 1 provided herein in a cycle, wherein the cycle comprises administering the formulation to deliver Compound 1 in a dose of about 0.1 mg to about 5 mg on days 1 to 5 and 15 to 19 of a 28 day cycle. In one embodiment, provided herein is a method of treating of MDS by administering to a subject a formulation of Compound 1 provided herein in a cycle, wherein the cycle comprises administering the formulation to deliver Compound 1 in a dose of about 0.5 mg to about 5 mg on days 1 to 5 and 15 to 19 of a 28 day cycle.

Patient Population

In certain embodiments of the methods provided herein, the subject is an animal, preferably a mammal, more preferably a non-human primate. In particular embodiments, the subject is a human. The subject can be a male or female subject.

Particularly useful subjects for the methods provided herein include human cancer patients, for example, those who have been diagnosed with leukemia, including acute myeloid leukemia, acute lymphocytic leukemia, chronic myelogenous leukemia, and chronic myelogenous leukemia. In certain embodiments, the subject has not been diagnosed with acute promyelocytic leukemia.

In some embodiments, the subject has a higher than normal blast population. In some embodiments, the subject has a blast population of at least 10%. In some embodiments, the subject has a blast population of between 10 and 15%. In some embodiments, the subject has a blast population of at least 15%. In some embodiments, the subject has a blast population of between 15 and 20%. In some embodiments, the subject has a blast population of at least 20%. In some embodiments, the subject has a blast population of about 10-15%, about 15-20%, or about 20-25%. In other embodiments, the subject has a blast population of less than 10%. In the context of the methods described herein, useful subjects having a blast population of less than 10% includes those subjects that, for any reason according to the judgment of the skilled practitioner in the art, are in need of treatment with a compound provided herein, alone or in combination with a second active agent.

In some embodiments, the subject is treated based on the Eastern Cooperative Oncology Group (ECOG) performance status score of the subject for leukemia. ECOG performance status can be scored on a scale of 0 to 5, with 0 denoting asymptomatic; 1 denoting symptomatic but completely ambulant; 2 denoting symptomatic and <50% in bed during the day; 3 denoting symptomatic and >50% in bed, but not bed bound; 4 denoting bed bound; and 5 denoting death. In some embodiments, the subject has an ECOG performance status score of 0 or 1. In some embodiments, the subject has an ECOG performance status score of 0. In some embodiments, the subject has an ECOG performance status score of 1. In other embodiments, the subject has an ECOG performance status score of 2.

In certain embodiments, the methods provided herein encompass the treatment of subjects who have not been previously treated for leukemia. In some embodiments, the subject has not undergone allogeneic bone marrow transplantation. In some embodiments, the subject has not undergone a stem cell transplantation. In some embodiments, the subject has not received hydroxyurea treatment. In some embodiments, the subject has not been treated with any investigational products for leukemia. In some embodiments, the subject has not been treated with systemic glucocorticoids.

In other embodiments, the methods encompass treating subjects who have been previously treated or are currently being treated for leukemia. For example, the subject may have been previously treated or are currently being treated with a standard treatment regimen for leukemia. The subject may have been treated with any standard leukemia treatment regimen known to the practitioner of skill in the art. In certain embodiments, the subject has been previously treated with at least one induction/reinduction or consolidation AML regimen. In some embodiments, the subject has undergone autologous bone marrow transplantation or stem cell transplantation as part of a consolidation regimen. In some embodiments, the bone marrow or stem cell transplantation occurred at least 3 months prior to treatment according to the methods provided herein. In some embodiments, the subject has undergone hydroxyurea treatment. In some embodiments, the hydroxyurea treatment occurred no later than 24 hours prior to treatment according to the methods provided herein. In some embodiments, the subject has undergone prior induction or consolidation therapy with cytarabine (Ara-C). In some embodiments, the subject has undergone treatment with systemic glucocorticosteroids. In some embodiments, the glucocorticosteroid treatment occurred no later 24 hours prior to treatment according to the methods described herein. In other embodiments, the methods encompass treating subjects who have been previously treated for cancer, but are non-responsive to standard therapies.

Also encompassed are methods of treating subjects having relapsed or refractory leukemia. In some embodiments, the subject has been diagnosed with a relapsed or refractory AML subtype, as defined by the World Health Organization (WHO). Relapsed or refractory disease may be de novo AML or secondary AML, e.g., therapy-related AML (t-AML).

In some embodiments, the methods provided herein are used to treat leukemia, characterized by presence of a mutant allele of IDH2. In one embodiment, the mutant allele of IDH2 is IDH2 R140Q or R172K.

In some embodiments, the methods provided herein are used to treat AML, characterized by presence of a mutant allele of IDH2. In one embodiment, the mutant allele of IDH2 is IDH2 R140Q or R172K.

Thus, treatment with a compound provided herein could provide an alternative for patients who do not respond to other methods of treatment. In some embodiments, such other methods of treatment encompass treatment with Gleevec® (imatinib mesylate). In some embodiments, provided herein are methods of treatment of Philadelphia chromosome positive chronic myelogenous leukemia (Ph+CML). In some embodiments, provided herein are methods of treatment of Gleevec® (imatinib mesylate) resistant Philadelphia chromosome positive chronic myelogenous leukemia (Ph+CML).

In some embodiments, the methods provided herein are used to treat drug resistant leukemias, such as CML. Thus, treatment with a compound provided herein could provide an alternative for patients who do not respond to other methods of treatment. In some embodiments, such other methods of treatment encompass treatment with Gleevec® (imatinib mesylate). In some embodiments, provided herein are methods of treatment of Ph+CML. In some embodiments, provided herein are methods of treatment of Gleevec® (imatinib mesylate) resistant Ph+CML.

Also encompassed are methods of treating a subject regardless of the subject's age, although some diseases or disorders are more common in certain age groups. In some embodiments, the subject is at least 18 years old. In some embodiments, the subject is more than 18, 25, 35, 40, 45, 50, 55, 60, 65, or 70 years old. In other embodiments, the subject is less than 65 years old. In some embodiments, the subject is less than 18 years old. In some embodiments, the subject is less than 18, 15, 12, 10, 9, 8 or 7 years old.

In some embodiments, the methods may find use in subjects at least 50 years of age, although younger subjects could benefit from the method as well. In other embodiments, the subjects are at least 55, at least 60, at least 65, and at least 70 years of age. In another embodiment, the subject has a cancer with adverse cytogenetics. “Adverse cytogenetics” is defined as any nondiploid karyotype, or greater than or equal to 3 chromosomal abnormalities. In another embodiment, the subjects are at least 60 years of age and have a cancer with adverse cytogenetics. In another embodiment, the subjects are 60-65 years of age and have a cancer with adverse cytogenetics. In another embodiment, the subjects are 65-70 years of age and have a cancer with adverse cytogenetics.

In certain embodiments, the subject treated has no history of myocardial infarction within three months of treatment according to the methods provided herein. In some embodiments, the subject has no history of cerebrovascular accident or transient ischemic attack within three months of treatment according to the methods provided herein. In some embodiments, the subject has no suffered no thromboembelic event, including deep vein thrombosis or pulmonary embolus, within 28 days of treatment according to the methods provided herein. In other embodiments, the subject has not experienced or is not experiencing uncontrolled disseminated intravascular coagulation.

Because subjects with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual subject with cancer.

It will be appreciated that every suitable combination of the compounds provided herein with one or more of the aforementioned compounds and optionally one or more further pharmacologically active substances is contemplated herein.

Evaluation of Activity

Standard physiological, pharmacological and biochemical procedures are available for testing the compounds to identify those that possess the desired activity.

Such assays include, for example, cell based assays, including the assay described in the Example section.

Embodiments provided herein may be more fully understood by reference to the following examples. These examples are meant to be illustrative of pharmaceutical compositions and dosage forms provided herein, but are not in any way limiting.

### Examples

The following Examples are presented by way of illustration, not limitation. The following abbreviations are used in descriptions and examples.
D5W—Dextrose 5% in Water
DSC—Differential scanning calorimetry
FDM—Freeze-drying microscope
HA—Human Albumin
PVP—polyvinylpyrrolidone (PVP)
RH—relative humidity
rHSA—Recombinant Human Serum Albumin
tBA or TBA—tert-butyl alcohol

“Compound 1” or “API” in the Examples herein refers to polymorph Form C of 2-(4-chlorophenyl)-N-((2-(2,6-dioxopiperidin-3-yl)-1-oxoisoindolin-5-yl)methyl)-2,2-difluoroacetamide. The physical and chemical properties of 2-(4-chlorophenyl)-N-((2-(2,6-dioxopiperidin-3-yl)-1-oxoisoindolin-5-yl)methyl)-2,2-difluoroacetamide are summarized in Table 1. Other forms of Compound 1, including Form A, Form B, Form D, Form F and amorphous form can be used in the formulations provided herein.

TABLE 1
Summary of physical and chemical properties of 2-(4-chlorophenyl)-N-((2-(2,6-
dioxopiperidin-3-yl)-1-oxoisoindolin-5-yl)methyl)-2,2-difluoroacetamide

| | |
|---|---|
| Structure | 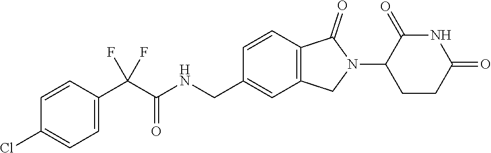 [SMILES: O=C1CCC(N2Cc3cc(CNC(=O)C(F)(F)c4ccc(Cl)cc4)ccc3C2=O)C(=O)N1] |
| Molecular Formula | C₂₂H₁₈ClF₂N₃O₄ |
| Molecular Weight | 461.85 |
| Log D | cLogP = 2.18 (Log D not measured due to solubility) |
| pKa | cpKa = 10.66 (Not measured due to low stability above pH 7) |
| Melting Point | 234° C. (Form C) |
| Appearance | White powder |
| Solubility | Practically insoluble in water (≤1 μg/ml across pH range of 1-8) |
| Solid State Stability | DS is physically stable under all storage conditions. |
| Solution Stability | DS is not stable in solution at pH of 5.0 or above. Hydrolysis is |
| | the major degradation pathway. |
| Hygroscopicity | Not hygroscopic |
| Pharmaceutical Form | Crystalline; Anhydrous; five polymorph forms |

“Related impurities” in the Examples herein encompass the following compounds:

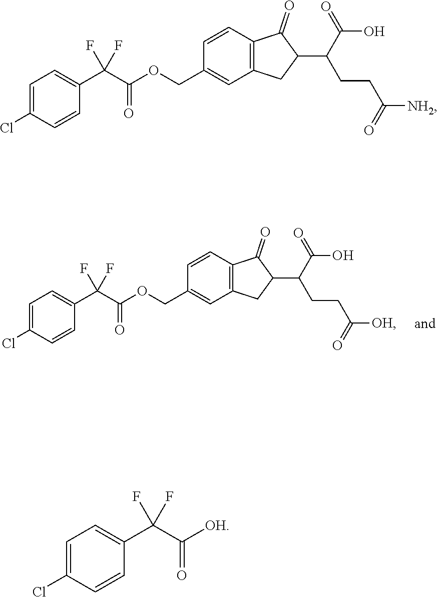 [SMILES: NC(=O)CCC(C(=O)O)C1Cc2cc(COC(=O)C(F)(F)c3ccc(Cl)cc3)ccc2C1=O ; O=C(O)CCC(C(=O)O)C1Cc2cc(COC(=O)C(F)(F)c3ccc(Cl)cc3)ccc2C1=O ; O=C(O)C(F)(F)c1ccc(Cl)cc1]

### Example 1: Formulation Screen for Mannitol Formulations

In a formulation screen, 14 prototype formulations were prepared with the following excipients: mannitol, trehalose, lactose, polyvinylpyrrolidone (PVP), and mannitol+trehalose. To balance the solubility of both API and the excipients in the solution, a solvent system of a 60:40 (v/v) tert-butyl alcohol (tBA)+pH 4 citrate buffer solution, or 50:50 (v/v) water for injection (WFI)+tBA was used.

TABLE 2
Formulation screen

| | | | | | | 10 mM | | | |
|---|---|---|---|---|---|---|---|---|---|
| | | | | | | citric | Formic | | |
| | API | Mannitol | Trehalose | Lactose | PVP | buffer | acid | WFI | |
| # | (mg/ml) | (mg/ml) | (mg/ml) | (mg/ml) | (mg/ml) | (% v/v) | (mg/ml) | (% v/v) | tBA |
| 1 | 0.1 | 25 | — | — | — | 40 | — | — | 60 |
| 2 | 0.1 | — | 25 | — | — | 40 | — | — | 60 |
| 3 | 0.1 | — | — | 25 | — | 40 | — | — | 60 |
| 4 | 0.1 | — | — | — | 25 | 40 | — | — | 60 |
| 5 | 0.1 | 25 | 10 | — | — | 4 | — | — | 60 |
| 6 | 0.1 | 50 | — | — | — | — | 0.125 | 50 | 50 |
| 7 | 0.1 | — | 50 | — | — | — | 0.125 | 50 | 50 |
| 8 | 0.1 | — | — | 50 | — | — | 0.125 | 50 | 50 |
| 9 | 0.1 | — | — | — | 50 | — | 0.125 | 50 | 50 |
| 10 | 0.1 | 25 | 25 | — | — | — | 0.125 | 50 | 50 |
| 11a | 0.1 | — | 20 | — | — | 40 | — | — | 60 |
| 11b | 0.1 | — | 20 | — | — | 40 | — | — | 60 |
| 12 | 0.1 | — | 10 | — | — | 40 | — | — | 60 |
| 13 | 0.1 | 10 | — | — | — | 40 | — | — | 60 |
| 14 | 0.1 | — | — | — | 10 | 40 | — | — | 60 |

Table 2A below provides lyophilization cycle for formulations provided above in Table 2.

TABLE 2A

| | Shelf Temp. | Hold/Ramp | Ramping | |
|---|---|---|---|---|
| | Setpoint | Time | Rate | |
| Step | (° C.) | (minutes) | (° C./min) | Pressure Setpoint |
| Product | 20 | 30 | | Evac. To 550 mTorr |
| Loading/ | | 140 | 0.5 | to ensure chamber is |
| Freezing | | | | airtight |
| Freezing | −50 | 180 | | |
| | −20 | 60 | 0.5 | |
| | −20 | 180 | | |
| | −50 | 60 | 0.5 | |
| | −50 | 180 | | |
| | −50 | 30 | | 50 mTorr |
| Primary | −25 | 50 | 0.5 | |
| Drying | −25 | 3900 | | |
| Secondary | 40 | 260 | 0.25 | |
| Drying | 40 | 600 | | |
| | 20 | 40 | 0.5 | |
| Stoppering | 20 | | | Backfill nitrogen to |
| | | | | ~600 mTorr |

The lyophilized formulations containing mannitol or trehalose showed superior stability and reconstitution time than other prototype formulations.

Mannitol and trehalose levels of 5 and 8 mg/ml were evaluated in prototype formulations described in Table 3 below. The formulations described in Table 3 were prepared as follows:
Citric acid monohydrate and sodium citrate dihydrate were dissolved in WFI to achieve a solution of 10 mM pH 4 citrate buffer.
Mannitol or trehalose were added to the buffer solution to dissolve completely.
tBA was added to the buffer solution to achieve a 60:40 tBA/buffer mixture.
The drug substance was added to the tBA/buffer mixture and mixed to achieve a target concentration of 0.1 mg/ml.
The bulk solution was filtered by a 0.22 μm PVDF filter and filled into a 20 cc glass vials at 10 ml/vial.
The vials were partially stoppered and lyophilized using a conservative lyophilization cycle.

TABLE 3
Formulations with mannitol/trehalose

| | | | | 10 mM citric | Fill | Vial |
|---|---|---|---|---|---|---|
| | API | Mannitol | Trehalose | buffer + TBA | volume | size |
| # | (mg/ml) | (mg/ml) | (mg/ml) | (% v/v) | (mL) | (mL) |
| 15 | 0.1 | 5 | — | 40 + 60 | 10 | 20 |
| 16 | 0.1 | 8 | — | 40 + 60 | 10 | 20 |
| 17 | 0.1 | — | 5 | 40 + 60 | 10 | 20 |
| 18 | 0.1 | — | 8 | 40 + 60 | 10 | 20 |
| 19 | — | 8 | 10 | 40 + 60 | 10 | 20 |

The finished drug products were crimped and tested for properties, such as appearance, color, foreign matter, residual moisture, residual tBA, related impurities and reconstitution. The lyophilized vials were also put on stability at 25° C./60% RH and 40° C./75% RH conditions for up to 6 months and tested for properties, such as appearance, color, foreign matter, residual moisture, residual tBA, related impurities and reconstitution.

The lyophilized cake (1 mg/vial in a 20 cc vial) was reconstituted with 5 ml diluent to achieve a clear and colorless solution at a concentration of 0.2 mg/ml.

The reconstitution diluent was a solution of PEG400, ethanol, and water for injection mixture at a volume ratio of 50:10:40 with a drug solubility of 0.33 mg/ml. The reconstitution diluent was prepared by mixing PEG400, ethanol and WFI together in the amounts provided in Table 4 below.

TABLE 4
Reconstitution diluent composition

| Material | Composition (g/mL) | Composition (g/vial)^{b} |
|---|---|---|
| PEG 400 | 0.565 | 5.65 |
| Ethanol | 0.079 | 0.79 |
| Water for Injection | 0.400 | 4.00 |
| (WFI) | | |

^{b}bulk solution density = 0.898 g/ml

The reconstituted solution was filtered through 0.22 μm PVDF filter, filled into a 20 cc vial at 10 ml/vial, stoppered and crimped.

Tables 5 to 12 below provide results of stability evaluation for the lyophilized and reconstituted products at 25° C./60% RH and 40° C./75% RH conditions for up to 3 months.

TABLE 5
Mannitol concentration of 5 mg/ml at 25° C./60% RH

| 25° C./60% RH | T0 | 1 month | 3 month |
|---|---|---|---|
| Appearance (lyo) | * | Conforms | Conforms |
| Color | * | White | White |
| Foreign Matter | * | N/A | N/A |
| Appearance of | * | Clear & | Clear & |
| Reconstituted Product | | Colorless | Colorless |
| Reconstitution Time (s) | * | 241 | 286 |
| pH | * | NP | NP |
| Container Appearance | * | N/A | N/A |
| Water Content | 0.25% | 0.28% | 0.10% |
| Assay (UPLC) | 103.2% | 103.0% | 101.8% |
| Related Impurities (UPLC) | ND | ND | ND |
| Total impurities | 0.00% | 0.00% | 0.00% |
| Residual TBA | 0.14% | | |

TABLE 6
Mannitol concentration of 5 mg/ml at 40° C./75% RH

| 40° C./75% RH | T0 | 2 weeks | 1 month | 3 months |
|---|---|---|---|---|
| Appearance (lyo) | * | | Conforms | Conforms |
| Color | * | | White | White |
| Foreign Matter | * | | N/A | N/A |
| Appearance of | * | | Clear & | Clear & |
| Reconstituted Product | | | Colorless | Colorless |
| Reconstitution | * | | 199 | 181 |
| Time (s) | | | | |
| pH | * | | NP | NP |
| Container | * | | N/A | N/A |
| Appearance | | | | |
| Water Content | 0.25% | | 0.41% | 0.65% |
| Assay (UPLC) | 103.2% | 101.6% | 102.6% | 102.7% |
| Impurity at relative | ND | 0.06% | ND | ND |
| retention time 0.48 | | | | |
| Hydrolysis 1 | ND | 0.07% | ND | ND |
| Total impurities | 0.00% | 0.13% | 0.00% | 0.00% |
| Residual TBA | 0.14% | | | |

TABLE 7
Mannitol concentration of 8 mg/ml at 25° C./60% RH

| 25° C./60% RH | T0 | 1 month | 3 months |
|---|---|---|---|
| Appearance (lyo) | * | Conforms | Conforms |
| Color | * | White | White |
| Foreign Matter | * | N/A | N/A |
| Appearance of | * | Clear & | Clear & |
| Reconstituted Product | | Colorless | Colorless |
| Reconstitution Time (s) | * | 275 | 257 |
| pH | * | NP | NP |
| Container Appearance | * | N/A | N/A |
| Water Content | 0.19% | 0.23% | 0.11% |
| Assay (UPLC) | 102.9% | 102.0% | 102.3% |
| Related Impurities | ND | ND | ND |
| (UPLC) | | | |
| Total impurities | 0.00% | 0.00% | 0.00% |
| Residual TBA | 0.09% | | |

TABLE 8
Mannitol concentration of 8 mg/ml at 40° C./75% RH

| 40° C./75% RH | T0 | 2 weeks | 1 month | 3 months |
|---|---|---|---|---|
| Appearance (lyo) | * | | Conforms | Conforms |
| Color | * | | White | White |
| Foreign Matter | * | | N/A | N/A |
| Appearance of | * | | Clear & | Clear & |
| Reconstituted Product | | | Colorless | Colorless |
| Reconstitution | * | | 207 | 163 |
| Time (s) | | | | |
| pH | * | | NP | NP |
| Container Appearance | * | | N/A | N/A |
| Water Content | 0.19% | | 0.27% | 0.35% |
| Assay (UPLC) | 102.9% | 101.0% | 102.4% | 102.6% |
| Related impurities | ND | 0.06% | ND | ND |
| (UPLC) | | | | |
| Total impurities | 0.00% | 0.00% | 0.00% | 0.00% |
| Residual TBA | 0.09% | | | |

TABLE 9
Trehalose concentration of 5 mg/ml at 25° C./60% RH

| 25° C./60% RH | T0 | 1 month | 3 months |
|---|---|---|---|
| Appearance (lyo) | * | Conforms | Conforms |
| Color | * | White | White |
| Foreign Matter | * | N/A | N/A |
| Appearance of | * | Clear & | Clear & |
| Reconstituted Product | | Colorless | Colorless |
| Reconstitution Time (s) | * | 219 | 200 |
| pH | * | NP | NP |
| Container Appearance | * | N/A | N/A |
| Water Content | 0.17% | 0.32% | 0.34% |
| Assay (UPLC) | 103.2% | 102.6% | 102.7% |
| Related Impurities | ND | ND | ND |
| (UPLC) | | | |
| Total impurities | 0.00% | 0.00% | 0.00% |
| Residual TBA | 0.88% | | |

TABLE 10
Trehalose concentration of 5 mg/ml at 40° C./75% RH

| 40° C./75% RH | T0 | 2 weeks | 1 month | 3 months |
|---|---|---|---|---|
| Appearance (lyo) | * | | Conforms | Conforms |
| Color | * | | White | White |
| Foreign Matter | * | | N/A | N/A |
| Appearance of | * | | Clear & | Clear & |
| Reconstituted Product | | | Colorless | Colorless |
| Reconstitution | * | | 174 | 177 |
| Time (s) | | | | |
| pH | * | | NP | NP |
| Container Appearance | * | | N/A | N/A |
| Water Content | 0.17% | | 0.38% | 0.47% |
| Assay (UPLC) | 103.2% | 101.8% | 102.6% | 102.7% |
| Related impurities | ND | ND | ND | ND |
| Total impurities | 0.00% | 0.00% | 0.00% | 0.00% |
| Residual TBA | 0.88% | | | |

TABLE 11
Trehalose concentration of 8 mg/ml at 25° C./60% RH

| 25° C./60% RH | T0 | 1 month | 3 months |
|---|---|---|---|
| Appearance (lyo) | * | Conforms | Conforms |
| Color | * | White | White |
| Foreign Matter | * | N/A | N/A |
| Appearance of | * | Clear & | Clear & |
| Reconstituted Product | | Colorless | Colorless |
| Reconstitution Time (s) | * | 168 | 257 |
| pH | * | NP | NP |
| Container Appearance | * | N/A | N/A |
| Water Content | 0.12% | 0.33% | 0.25% |
| Assay (UPLC) | 102.7% | 102.5% | 102.9% |
| Impurity at relative | ND | ND | ND |
| retention time 0.50 | | | |
| Hydrolysis 1 | ND | ND | 0.52% |
| Total impurities | 0.00% | 0.00% | 0.63% |
| Residual TBA | 0.99% | | |

TABLE 12
Trehalose concentration of 8 mg/ml at 40° C./75% RH

| 40° C./75% RH | T0 | 2 weeks | 1 month | 3 months |
|---|---|---|---|---|
| Appearance (lyo) | * | | Conforms | Conforms |
| Color | * | | White | White |
| Foreign Matter | * | | N/A | N/A |
| Appearance of | * | | Clear & | Clear & |
| Reconstituted Product | | | Colorless | Colorless |
| Reconstitution Time (s) | * | | 144 | 165 |
| pH | * | | NP | NP |
| Container Appearance | * | | N/A | N/A |
| Water Content | 0.12% | | 0.27% | 0.53% |
| Assay (UPLC) | 102.7% | 101.3% | 101.6% | 102.1% |
| Related impurities | ND | ND | ND | ND |
| Total impurities | 0.00% | 0.00% | 0.00% | 0.00% |
| Residual TBA | 0.99% | | Average | Average |

The formulation with mannitol concentration of 8 mg/ml was selected as it provided acceptable cake appearance as well as acceptable reconstitution time and solution appearance. Table 13 below provides the composition for the final formulation.

TABLE 13
Drug Product Formulation Compositions (1 mg/vial in 20 cc vial)

| | Composition | Composition |
|---|---|---|
| | of Bulk | of Finished |
| Material | Solution^{b} (mg/mL) | Drug Product (mg/vial)^{b} |
| Compound 1 | 0.10 | 1.0 |
| Mannitol | 8.0 | 80.0 |
| Citric Acid Monohydrate | 0.524 | 5.24 |
| Sodium Citrate Dihydrate | 0.44 | 4.4 |
| Tert-butyl Alcohol (tBA)^{a} | 465.0 | Removed upon drying |
| Water for Injection (WFI) | 400.0 | Removed upon drying |

^{a}tBA density = 0.775 g/ml. tBA: water = 60:40 v/v
^{b}bulk solution density = 0.898 g/ml

The formulation demonstrated acceptable stability at 25° C./60% RH and 40° C./75% RH storage condition for 3 months as shown in Tables 7 and 8, respectively.

### Example 2: Formulation Screen for Human Albumin Formulations

In a formulation screen, 16 formulations of Compound 1 (Formulations 1-16) were prepared with human albumin or recombinant human albumin. Tables 14 and 15 below provide compositions for each of the formulation in bulk solution.

For each of Formulations 1-16, mass of each component in the vial is provided in Tables 16 and 17 below. For each of Formulations 1-16, mass fraction of each component in the lyophilized product s provided in Tables 18 and 19 below.

TABLE 14

| Formulation No. | 1 | 2 | 3 | 4 | 5 | 6 | 7 | 8 |
|---|---|---|---|---|---|---|---|---|
| Compound 1 Concentration (μg/mL) | 25 | 50 | 100 | 200 | 200 | 200 | 50 | 100 |
| Human Albumin Concentration (mg/mL) | 50 | 50 | 50 | 100 | 100 | 100 | 100 | 100 |
| Albumin/Compound 1 Ratio | 2000 | 1000 | 500 | 500 | 500 | 500 | 2000 | 1000 |
| Sucrose Concentration (mg/mL) | 0 | 0 | 0 | 0 | 40 | 0 | 0 | 0 |
| Citric Acid Concentration (mM) | 18.75 | 18.75 | 18.75 | 18.75 | 18.75 | 18.75 | 20 | 20 |
| pH prior to formic acid addition | 5 | 5 | 5 | 5 | 5 | 5 | | |
| Formic acid Concentration (μg/mL) | 0.20 | 0.41 | 0.81 | 1.63 | 1.63 | 1.63 | 0.41 | 0.81 |
| Sodium N-acetyltryptophanate Conc. (mM) | 4.0 | 4.0 | 4.0 | 8.0 | 8.0 | 0.0 | 8.0 | 8.0 |
| Sodium caprylate Conc. (mM) | 4.0 | 4.0 | 4.0 | 8.0 | 8.0 | 0.0 | 8.0 | 8.0 |
| pH of fully formulated solution | | | | | | | 4.7 | 4.8 |
| Tonicity of fully formulated solution | | | | | | | 163 | 153 |
| (mOsm/kg) | | | | | | | | |
| Vial size (cc) | 50 | 50 | 50 | 50 | 50 | 50 | 50 | 50 |
| Fill Volume (mL) | 24 | 24 | 24 | 12 | 12 | 12 | 12 | 12 |
| Reconstituted Volume (mL) | 24 | 24 | 24 | 24 | 24 | 24 | 24 | 24 |
| Reconstitution media | 0.9% | 0.9% | 0.9% | 0.9% | | 0.9% | 0.9% | 0.9% |
| | NaCl | NaCl | NaCl | NaCl | | NaCl | NaCl | NaCl |
| Volume of WFI to reconstitute (mL) | 22.8 | 22.8 | 22.8 | 22 | 22 | 22 | 22.8 | 22.8 |
| pH of reconstituted solution | | | | | | | 4.7 | 4.8 |
| Tonicity of reconstituted solution | | | | | | | 358 | 357 |
| (mOsm/kg) | | | | | | | | |

TABLE 15

| Formulation No. | 9 | 10 | 11 | 12 | 13 | 14 | 15 | 16 |
|---|---|---|---|---|---|---|---|---|
| Compound 1 Concentration (μg/mL) | 100 | 50 | 200 | 200 | 50 | 100 | 50 | 120 |
| Human Albumin Concentration (mg/mL) | 100 | 50 | 100 | 100 | 50 | 100 | 50 | 100 |
| Albumin/Compound 1 Ratio | 1000 | 1000 | 500 | 500 | 1000 | 1000 | 1000 | 833 |
| Sucrose Concentration (mg/mL) | 134 | 80 | 0 | 0 | 68.5 | 137 | 60 | 120 |
| Citric Acid Concentration (mM) | 20 | 22.5 | 20 | 20 | 22.5 | 20 | 22.5 | 40 |
| pH prior to formic acid addition | | | | | | | | 4.2 |
| Formic acid Concentration (μg/mL) | 0.81 | 0.41 | 1.63 | 1.63 | 0.41 | 0.81 | 0.41 | 0.98 |
| Sodium N-acetyltryptophanate Conc. (mM) | 8.0 | 4.0 | 0.0 | 8.0 | 4.0 | 8.0 | 4.0 | 8.0 |
| Sodium caprylate Conc. (mM) | 8.0 | 4.0 | 4.0 | 8.0 | 4.0 | 8.0 | 4.0 | 8.0 |
| pH of fully formulated solution | 4.8 | 4.8 | 4.5 | 4.5 | | | | |
| Tonicity of fully formulated solution | 732 | 387 | 322 | 182 | | | | |
| (mOsm/kg) | | | | | | | | |
| Vial size (cc) | 50 | 50 | 20 | 20 | 100 | 50 | 50 | 100 |
| Fill Volume (mL) | 12 | 24 | 6 | 6 | 24 | 12 | 20 | 25 |
| Reconstituted Volume (mL) | 24 | 24 | 12 | 12 | 24 | 24 | 20 | 50 |
| Reconstitution media | WFI | WFI | 0.9% | 0.9% | WFI | WFI | WFI | WFI |
| | | | NaCl | NaCl | | | | |
| Volume of WFI to reconstitute (mL) | 22 | 22 | 11.4 | 11.4 | 22.3 | 22.3 | 18.6 | 46.5 |
| pH of reconstituted solution | 4.8 | 4.8 | 4.5 | 4.5 | | | | |
| Tonicity of reconstituted solution | 302 | 379 | 437 | 366 | | | | |
| (mOsm/kg) | | | | | | | | |

TABLE 16

| Formulation No. | 1 | 2 | 3 | 4 | 5 | 6 | 7 | 8 |
|---|---|---|---|---|---|---|---|---|
| Compound 1 per vial (mg) | 0.6 | 1.2 | 2.4 | 2.4 | 2.4 | 2.4 | 0.6 | 1.2 |
| Human Albumin per vial (mg) | 1200 | 1200 | 1200 | 1200 | 1200 | 1200 | 1200 | 1200 |
| Sucrose per vial (mg) | 0 | 0 | 0 | 0 | 480 | 0 | 0 | 0 |
| Citric acid per vial (mg) | 86.5 | 86.5 | 86.5 | 43.2 | 43.2 | 43.2 | 46.1 | 46.1 |
| Sodium chloride per vial (mg) | 50.8 | 50.8 | 50.8 | 50.8 | 50.8 | 50.8 | 50.8 | 50.8 |
| Sodium N-acetyltryptophanate | 25.8 | 25.8 | 25.8 | 25.8 | 25.8 | 0.0 | 25.8 | 25.8 |
| per vial (mg) | | | | | | | | |
| Sodium caprylate per vial (mg) | 16.0 | 16.0 | 16.0 | 16.0 | 16.0 | 0.0 | 16.0 | 16.0 |
| Total mass per vial (mg) | 1379.6 | 1380.2 | 1381.4 | 1338.2 | 1818.2 | 1296.5 | 1339.3 | 1339.9 |

TABLE 17

| Formulation No. | 9 | 10 | 11 | 12 | 13 | 14 | 15 | 16 |
|---|---|---|---|---|---|---|---|---|
| Compound 1 per vial (mg) | 1.2 | 2.4 | 1.2 | 1.2 | 1.2 | 1.2 | 1 | 3 |
| Human Albumin per vial (mg) | 1200 | 1200 | 600 | 600 | 1200 | 1200 | 1000 | 2500 |
| Sucrose per vial (mg) | 1608 | 1920 | 0 | 0 | 1644 | 1644 | 1200 | 3000 |
| Citric acid per vial (mg) | 46.1 | 103.7 | 23.1 | 23.1 | 103.7 | 46.1 | 86.5 | 192.1 |
| Sodium chloride per vial (mg) | 50.8 | 50.8 | 50.8 | 25.4 | 50.8 | 50.8 | 42.4 | 105.9 |
| Sodium N-acetyltryptophanate | 25.8 | 25.8 | 0.0 | 12.9 | 25.8 | 25.8 | 21.5 | 53.6 |
| per vial (mg) | | | | | | | | |
| Sodium caprylate per vial (mg) | 16.0 | 16.0 | 4.0 | 8.0 | 16.0 | 16.0 | 13.3 | 33.2 |
| Total mass per vial (mg) | 2947.9 | 3318.7 | 679.1 | 670.5 | 3041.5 | 2983.9 | 2364.6 | 5887.9 |

TABLE 18

| Formulation No. | 1 | 2 | 3 | 4 | 5 | 6 | 7 | 8 |
|---|---|---|---|---|---|---|---|---|
| Compound 1 per vial (% w/w) | 0.043 | 0.087 | 0.174 1 | 0.179 | 0.132 | 0.185 | 0.045 | 0.090 |
| Human Albumin per vial (% w/w) | 86.98 | 86.94 | 86.87 | 89.67 | 66.00 | 92.56 | 89.602 | 89.561 |
| Sucrose per vial (% w/w) | 0.000 | 0.000 | 0.000 | 0.000 | 26.400 | 0.000 | 0.000 | 0.000 |
| Citric acid per vial (% w/w) | 6.267 | 6.264 | 6.258 | 3.230 | 2.378 | 3.334 | 3.443 | 3.441 |
| Sodium chloride per vial (% w/w) | 3.685 | 3.684 | 3.681 | 3.800 | 2.797 | 3.922 | 3.797 | 3.795 |
| Sodium N-acetyltryptophanate | 1.867 | 1.866 | 1.864 | 1.924 | 1.416 | 0.000 | 1.923 | 1.922 |
| per vial (% w/w) | | | | | | | | |
| Sodium caprylate per vial (% w/w) | 1.156 | 1.156 | 1.155 | 1.192 | 0.878 | 0.000 | 16.0 | 16.0 |

TABLE 19

| Formulation No. | 9 | 10 | 11 | 12 | 13 | 14 | 15 | 16 |
|---|---|---|---|---|---|---|---|---|
| Compound 1 per vial (% w/w) | 0.041 | 0.072 | 0.177 1 | 0.179 | 0.039 | 0.040 | 0.042 | 0.051 |
| Human Albumin per vial (% w/w) | 40.707 | 36.159 | 88.354 | 89.481 | 39.454 | 40.216 | 42.291 | 42.460 |
| Sucrose per vial (% w/w) | 54.548 | 57.854 | 0.000 | 0.000 | 54.052 | 55.096 | 50.749 | 50.952 |
| Citric acid per vial (% w/w) | 1.564 | 3.126 | 3.395 | 3.438 | 3.411 | 1.545 | 3.656 | 3.263 |
| Sodium chloride per vial (% w/w) | 1.725 | 1.532 | 7.487 | 3.791 | 1.672 | 1.704 | 1.792 | 1.799 |
| Sodium N-acetyltryptophanate | 0.874 | 0.776 | 0.000 | 1.920 | 0.847 | 0.863 | 0.908 | 0.911 |
| per vial (% w/w) | | | | | | | | |
| Sodium caprylate per vial (% w/w) | 0.541 | 0.481 | 0.587 | 1.190 | 0.525 | 0.535 | 0.562 | 0.565 |

In Formulations 1-3 described above, the physical stability (recrystallization and precipitation of Compound 1) was examined at ratios of human albumin (HA): Compound 1 ranging from 500 to 2000. All formulations were made with the same citrate buffer, to the same pH and with the same human albumin concentration of 50 mg/mL to match typical albumin plasma concentrations in patients. All formulations were filled in 50 cc vials with 24 mL of solution and lyophilized using an aggressive freezing and drying cycle described in Table 20.

TABLE 20

| | Temperature | Duration | Pressure |
|---|---|---|---|
| | [° C.] | [minutes] | [mTorr] |
| Pre-cooling hold | 7 | 60 | Atmos. |
| Freezing ramp | −38 | 45 | Atmos. |
| Freezing hold | −38 | 240 | Atmos. |
| Vacuum equilibration | −38 | 10 | 350 |
| Primary drying ramp | −15 | 200 | 350 |
| Primary drying hold | −15 | 1500 | 350 |
| Secondard drying ramp | 25 | 270 | 350 |
| Secondary drying hold | 25 | 1500 | 350 |
| High temperature drying ramp | N/A | N/A | N/A |
| High temperature drying hold | N/A | N/A | N/A |
| Ramp to 25° C. | N/A | N/A | N/A |

It was observed that all bulk formulated but unfiltered solutions before vial filling were physically stable (Compound 1 did not precipitate as determined by loss on 0.2 μm filtration) for at least 90 hours at 4° C. It was also observed that the 1000 and 2000 HA:Compound 1 solutions were stable by this same test but at room temperature storage for at least 18 days and the 500 HA:Compound 1 solution was stable at room temperature storage for approximately 7 days. The reconstitution time for the lyophilized drug product vials was approximately 20 minutes for all formulations. The lyophilized and reconstituted drug products for all three formulations were physically stable for at least 7 days at both room temperature and 4° C. The lyophilized and reconstituted drug product for the 2000 HA:Compound 1 formulation was physically stable for at least 14 days at room temperature and 4° C. These experiments demonstrated physical stability of formulated HA and Compound 1 solutions for at least 7 days at HA:Compound 1 ratios of at least 500 and longer stability for HA:drug ratios for at least 1000.

Formulations 4-6 examined the effect of the additional excipient sucrose (to improve reconstitution time and long-term storage stability of the HA in the lyophilized product) and the removal of the HA stabilizers sodium N-acetyltryptophanate and sodium caprylate (to increase the solubility of Compound 1 in the HA by removing these competing hydrophobic additives) on the physical stability of formulations using a HA:Compound 1 ratio of 500. All formulations were made with the same citrate buffer and to the same pH. In this case, a human albumin concentration of 100 mg/mL was used in the bulk compounded solutions but the reconstitution of the lyophilized product vials was performed with twice the vial fill volume to bring the reconstituted HA concentration to 50 mg/mL to match typical albumin plasma concentrations in patients. All formulations were filled in 50 cc vials with 12 mL of solution and lyophilized using an aggressive freezing and drying cycle provided in Table 21.

TABLE 21

| | Temperature | Duration | Pressure |
|---|---|---|---|
| | [° C.] | [minutes] | [mTorr] |
| Pre-cooling hold | 7 | 60 | Atmos. |
| Freezing ramp | −38 | 45 | Atmos. |
| Freezing hold | −38 | 240 | Atmos. |
| Vacuum equilibration | −38 | 10 | 350 |
| Primary drying ramp | −15 | 200 | 350 |
| Primary drying hold | −15 | 1500 | 350 |
| Secondard drying ramp | 25 | 270 | 350 |
| Secondary drying hold | 25 | 1500 | 350 |
| High temperature drying ramp | N/A | N/A | N/A |
| High temperature drying hold | N/A | N/A | N/A |
| Ramp to 25° C. | N/A | N/A | N/A |

It was observed that all bulk formulated but unfiltered solutions before vial filling were physically stable (drug did not precipitate as determined by loss on 0.2 μm filtration) for at least 15 days at room temperature. The reconstitution time for the lyophilized drug product vials was approximately 20 minutes for all formulations. The lyophilized and reconstituted drug products for all three formulations were physically stable for at least 7 days at both room temperature and 4° C. This experiment confirmed that the addition of sucrose and the removal of the HA stabilizers did not impact the stability of formulated HA and Compound 1 solutions. In addition, these experiments demonstrated that formulated HA and Compound 1 solutions at HA:Compound 1 ratios of at least 500 are stable for at least 15 days.

Formulations 7 and 8 tested the chemical and physical stability on long term storage of lyophilized drug product vials containing HA:Compound 1 ratios of 1000 and 2000. Both formulations were made with the same citrate buffer and to the same pH. A human albumin concentration of 100 mg/mL was used in the bulk compounded solutions but the reconstitution of the lyophilized product vials was performed with twice the vial fill volume to bring the reconstituted HA concentration to 50 mg/mL to match typical albumin plasma concentrations in patients. All formulations were filled in 50 cc vials with 12 mL of solution and lyophilized using an aggressive freezing and drying cycle provided in Table 22.

TABLE 22

| | Temperature | Duration | Pressure |
|---|---|---|---|
| | [°C .] | [minutes] | [mTorr] |
| Pre-cooling hold | 5 | 60 | Atmos. |
| Freezing ramp | −55 | 60 | Atmos. |
| Freezing hold | −55 | 240 | Atmos. |
| Vacuum equilibration | −55 | 10 | 350 |
| Primary drying ramp | −15 | 120 | 350 |
| Primary drying hold | −15 | 1255 | 350 |
| Secondard drying ramp | 25 | 120 | 350 |
| Secondary drying hold | 25 | 600 | 350 |
| High temperature drying ramp | 60 | 30 | 100 |
| High temperature drying hold | 60 | 1320 | 100 |
| Ramp to 25° C. | 25 | Uncontrolled | 100 |

The lyophilized and stoppered dry product vials were placed on storage stability at 3 storage conditions: 1) 5° C., 2) 25° C. and 60% RH, and 3) 40° C. and 75% RH. Samples were removed and reconstituted at 1 week, 2 week, 1 month, 2 month and 3 month time points. All lyophilized vials were reconstituted with 0.9% sodium chloride for injection, USP. The physical stability was assayed by loss of potency on filtration and the chemical stability was assayed by potency of the Compound 1 drug substance and the fraction of the two related impurities. The aggregation stability of the HA was assessed by size exclusion chromatography. In addition. the physical and chemical stability of Compound 1 in the reconstituted solutions held at 4° C. was tested. It was observed that both lyophilized formulations lost no more than 2% potency over 3 months at both the 25° C. and 40° C. storage conditions and there was no quantifiable amounts of related impurities formed over 3 months at all storage conditions. In addition, there was no more than 2% loss of potency on filtration at the 40° C. storage condition after 3 month of storage, and less at the 5° C. and 25° C. conditions. The reconstituted solutions lost no more than 1% potency and the related impurities increased by no more than 1% after storage for 9 weeks at 4° C. in the reconstituted state. This experiment demonstrated the long term and accelerated storage stability of lyophilized drug product vials containing HA and Compound 1 formulations with HA:drug ratios of 1000 and 2000. It also demonstrated that the reconstituted solutions remained chemically and physically stable for at least 9 weeks when stored at 4° C.

Formulations 9 and 10 tested the chemical and physical stability on long term storage of lyophilized drug product vials containing HA:Compound 1 ratios of 500 and 1000 and stabilized by sucrose. For formulation 9, a human albumin concentration of 100 mg/mL was used in the bulk compounded solution but the reconstitution of the lyophilized product vials was performed with twice the vial fill volume to bring the reconstituted HA concentration to 50 mg/mL to match typical albumin plasma concentrations in patients. For formulation 10, a human albumin concentration of 50 mg/mL was used in the bulk compounded solution and was reconstituted with the same volume as the fill volume. Sucrose was added to both formulations so that the reconstituted formulations resulted in isotonic solutions. Formulation 9 was filled in 50 cc vials with 24 mL of solution and formulation 10 was filled in 50 cc vials with 12 mL of solution. All vials were lyophilized using an aggressive freezing and drying cycle provided in Table 23.

TABLE 23

| | Temperature | Duration | Pressure |
|---|---|---|---|
| | [° C.] | [minutes] | [mTorr] |
| Pre-cooling hold | 5 | 60 | Atmos. |
| Freezing ramp | −55 | 60 | Atmos. |
| Freezing hold | −55 | 240 | Atmos. |
| Vacuum equilibration | −55 | 10 | 350 |
| Primary drying ramp | −15 | 120 | 350 |
| Primary drying hold | −15 | 1835 | 350 |
| Secondard drying ramp | 25 | 120 | 350 |
| Secondary drying hold | 25 | 600 | 350 |
| High temperature drying ramp | 60 | 30 | 100 |
| High temperature drying hold | 60 | 1200 | 100 |
| Ramp to 25° C. | 25 | Uncontrolled | 100 |

The lyophilized and stoppered dry product vials were placed on storage stability at 3 storage conditions: 1) 5° C., 2) 25° C. and 60% RH, and 3) 40° C. and 75% RH. Samples were removed and reconstituted at 1 week, 2 week, 1 month, 2 month and 3 month time points. All lyophilized vials were reconstituted with 22 mL of water for injection, USP. The physical stability was assayed by loss of potency on filtration and the chemical stability was assayed by potency of Compound 1 drug substance and the fraction of the two related impurities. The aggregation stability of the HA was assessed by size exclusion chromatography. The physical and chemical stability of Compound 1 in the reconstituted solutions held at 4° C. was tested. It was found that both lyophilized formulations showed no measurable loss of potency over 3 months at all three storage conditions (an improvement over the formulations without sucrose) and there was no quantifiable amounts of related impurities forming over 3 months at all storage conditions. In addition, there was no more than 1% loss of potency on filtration at any storage condition after 3 months of storage (an improvement over the formulations without sucrose). The reconstituted solutions of formulation 9 lost 0.7% potency and the related impurities increased by 0.7% after storage for 8 weeks at 4° C. in the reconstituted state. Similarly, the reconstituted solutions of formulation 10 lost 0.4% potency and the related impurities increased by 0.6% after storage for 8 weeks at 4° C. in the reconstituted state. Both demonstrated increased stability compared to formulations without sucrose. This experiment demonstrated improved long term and accelerated storage stability of lyophilized drug product vials containing HA and Compound 1 formulations with HA:drug ratios of 500 and 1000 and stabilized with sucrose. It also demonstrated that the reconstituted solutions were more chemically and physically stable than the formulations without sucrose when stored at 4° C.

Formulations 11 and 12 tested the chemical and physical stability on long term storage of lyophilized drug product vials containing HA:Compound 1 ratios of 500 with two different types of HA. In formulation 11, a recombinantly-produced human albumin from Novozymes (Albucut, 10% rHSA solution) was used. In formulation 12, a human blood-sourced albumin (Grifols) was used. For both formulations, a human albumin concentration of 100 mg/mL was used in the bulk compounded solution but the reconstitution of the lyophilized product vials was performed with twice the vial fill volume to bring the reconstituted HA concentration to 50 mg/mL to match typical albumin plasma concentrations in patients. Both formulations were filled in 20 cc vials with 6 mL of solution. All vials were lyophilized using an aggressive freezing and drying cycle provided in Table 24.

TABLE 24

| | Temperature | Duration | Pressure |
|---|---|---|---|
| | [° C.] | [minutes] | [mTorr] |
| Pre-cooling hold | 5 | 60 | Atmos. |
| Freezing ramp | −55 | 60 | Atmos. |
| Freezing hold | −55 | 240 | Atmos. |
| Vacuum equilibration | −55 | 10 | 350 |
| Primary drying ramp | −15 | 120 | 350 |
| Primary drying hold | −15 | 1835 | 350 |
| Secondard drying ramp | 25 | 120 | 350 |
| Secondary drying hold | 25 | 600 | 350 |
| High temperature drying ramp | 60 | 30 | 100 |
| High temperature drying hold | 60 | 1200 | 100 |
| Ramp to 25° C. | 25 | Uncontrolled | 100 |

The lyophilized and stoppered dry product vials were placed on storage stability at 3 storage conditions: 1) 5° C., 2) 25° C. and 60% RH, and 3) 40° C. and 75% RH. Samples were removed and reconstituted at 1 week, 2 week, 1 month, 2 month and 3 month time points. All lyophilized vials were reconstituted with 11.4 mL of 0.9% sodium chloride for injection, USP. The physical stability was assayed by loss of potency on filtration and the chemical stability was assayed by potency of the Compound 1 drug substance and the fraction of the two related impurities. The aggregation stability of the HA was assessed by size exclusion chromatography. The physical and chemical stability of Compound 1 in the reconstituted solutions held at 4° C. was tested. It was found that both lyophilized formulations showed no measurable difference in loss of potency over 3 months at all three storage conditions and there was no quantifiable amounts of related impurities forming over 3 months at all storage conditions. This experiment demonstrated no difference in the stability of formulations made with recombinant human albumin compared with those made with human-sourced albumin.

Formulations 13 and 14 tested the reconstitution time of lyophilized drug product vials containing HA:Compound 1 ratios of 1000 but lyophilized using slower freezing and primary drying steps to improve the cake properties. For formulation 13, a human albumin concentration of 50 mg/mL was used in the bulk compounded solution and was reconstituted with the same volume as the fill volume. Formulation 13 was filled in 100 cc vials with 24 mL of solution to make a total drug content of 1.2 mg/vial. For formulation 14, a human albumin concentration of 100 mg/mL was used in the bulk compounded solution but the reconstitution of the lyophilized product vials was performed with twice the vial fill volume to bring the reconstituted HA concentration to 50 mg/mL to match typical albumin plasma concentrations in patients. Formulation 14 was filled in 50 cc vials with 12 mL of solution to make a total drug content of 1.2 mg/vial. Sucrose was added to each formulation at a concentration to make the final reconstituted product isotonic when reconstituted with water for injection. The lyophilization cycle was altered from previous formulations with a freezing ramp rate of 0.25° C./minute and a primary drying shelf temperature of 20° C. and a primary drying vacuum pressure of 100 mTorr. The lyopholization cycle is provided in Table 25.

TABLE 25

| | Temperature | Duration | Pressure |
|---|---|---|---|
| | [° C.] | [minutes] | [mTorr] |
| Pre-cooling hold | 5 | 60 | Atmos. |
| Freezing ramp | −45 | 200 | Atmos. |
| Freezing hold | −45 | 240 | Atmos. |
| Vacuum equilibration | −45 | 10 | 100 |
| Primary drying ramp | −20 | 100 | 100 |
| Primary drying hold | −20 | 2410 | 100 |
| Secondard drying ramp | 25 | 450 | 350 |
| Secondary drying hold | 25 | 720 | 350 |
| High temperature drying ramp | N/A | N/A | N/A |
| High temperature drying hold | N/A | N/A | N/A |
| Ramp to 25° C. | N/A | N/A | N/A |

Reconstitution times of both lyophilized formulations were shortened to about 5-7 minutes, a substantial improvement over the earlier lyophilization cycle conditions.

Formulation 15 was similar to formulation 13 but manufactured at a 5 liter scale (5 L of formulated bulk solution) to demonstrate scalability of the manufacturing process. The human albumin concentration was 50 mg/mL and the sucrose concentration was 60 mg/mL in the bulk compounded solution. The bulk solution was sterile filtered through a 0.2 micron polyvinylidene difluoride (PVDF) membrane filter and filled in 50 cc vials with 20 mL of solution to make a total drug content of 1.0 mg/vial. The lyophilization cycle was altered from previous formulations with a freezing ramp rate of 0.25° C./minute and a primary drying shelf temperature of 20° C. and a primary drying vacuum pressure of 75 mTorr to prevent cake collapse during the ice sublimation process. The lyopholization cycle is provided in Table 26.

TABLE 26

| | Temperature | Duration | Pressure |
|---|---|---|---|
| | [° C.] | [minutes] | [mTorr] |
| Pre-cooling hold | 5 | 60 | 500,000 |
| Freezing ramp | −45 | 200 | 500,000 |
| Freezing hold | −45 | 240 | 500,000 |
| Vacuum equilibration | −45 | 30 | 75 |
| Primary drying ramp | −20 | 100 | 75 |
| Primary drying hold | −20 | 9060 | 75 |
| Secondard drying ramp | 25 | 450 | 75 |
| Secondary drying hold | 25 | 720 | 75 |
| High temperature drying ramp | 60 | 120 | 75 |
| High temperature drying hold | 60 | 1200 | 75 |
| Ramp to 25° C. | 25 | 70 | 75 |

The resulting lyophilized product was stable on storage, reconstituted within 5-7 minutes and the reconstituted solution was both physically and chemically stable at 4° C. This experiment demonstrated that formulations of sterile product quality could be manufactured at a scale representative of clinical or commercial batch sizes.

Formulation 16 was produced to increase the overall dose of Compound 1 to 3.0 mg/vial. Formulation 16 was formulated at bulk compounded solution concentrations of 120 μg/mL of Compound 1, 100 mg/mL of human albumin and 1200 mg/mL sucrose and 40 mM citrate buffer. The pH of the HA plus citric acid solution prior to addition of the formic acid and Compound 1 is 4.2 in order to reduce the amount of sodium formate in the bulk compounded solution and therefore increase the removal of formic acid during lyophilization. The bulk solution was sterile filtered through a 0.2 micron polyethersulfone (PES) membrane filter and filled in 100 cc vials with 25 mL of solution to make a total drug content of 3.0 mg/vial. The lyophilization cycle was altered from previous formulations with a freezing ramp rate of 0.25° C./minute and a primary drying shelf temperature of 20° C., a primary drying vacuum pressure of 100 mTorr, and a high temperature drying step of 60° C. to remove residual formic acid. The lyophilization cycle is summarized in Table 27.

TABLE 27

| | Temperature | Duration | Pressure |
|---|---|---|---|
| | [° C.] | [minutes] | [mTorr] |
| Pre-cooling hold | 5 | 60 | Atmos. |
| Freezing ramp | −45 | 200 | Atmos. |
| Freezing hold | −45 | 240 | Atmos. |
| Vacuum equilibration | −45 | 10 | 100 |
| Primary drying ramp | −20 | 100 | 100 |
| Primary drying hold | −20 | 2410 | 100 |
| Secondard drying ramp | 25 | 450 | 200 |
| Secondary drying hold | 25 | 720 | 200 |
| High temperature drying ramp | 60 | 400 | 200 |
| High temperature drying hold | 60 | 720 | 200 |
| Ramp to 25° C. | 25 | uncontrolled | 350 |

The lyophilized vials were reconstituted with 45.6 mL of water for injection, USP to make a stable 60 μg/mL drug solution. Up to 6.0 mg of Compound 1 can be administered in a 100 mL infusion of this reconstituted solution.

Preparations for Formulations 7-12 is further described in detail in Example 3, and for Formulation 15 is described in Example 4.

### Example 3: Human Albumin Formulations

The following materials were used in preparation of Formulations 7-12:
Albumin (Human), 20 g 100 mL, Grifols, Lot No.IBAC5D8001
MilliQ water, 18.4 MOhms-cm and 4 ppb TOC
Nalgene Rapid-Flow sterile disposable bottle top filters with PES membrane,
Thermofisher Scientific, #295-3345
Formic acid 97%, Alfa Aesar, A13285
Sodium citrate, dihydrate, BDH, 8017-500G
Citric acid, anhydrous, Spectrum, CI133
Filter w/supor 13 mm, 0.2 μm, Pkg75, VWR/PALL
Lyophilization vials: Allergy Laboratories, Inc., 10 mL-20 mm, sterile glass vials

Additionally, in Formulation 11, Novozymes Albucut, 10% rHSA solution, Batch # RF002 was used.

The following equipment was used to prepare Formulations 7-12:
Silverson L5M-A High Shear Laboratory Mixer
Branson 2510 Bath sonicator
Thermo Haake K35 refrigerated recirculating water bath with a DC50 temperature controller
Virtis Genesis 25EL lyophilizer

Equipment Preparation: The Silverson mixer was cleaned by rinsing twice with water, followed by a rinse with 70% IPA, followed by a final rinse with MilliQ water of WFI. The chiller was set to 5° C. and recirculated through water bath.

I. Formulations with with 10% Human Albumin:

Formulation 7 (HA/Compound 1 (1000:1)) and 8 (HA/Compound 1 (2000:1))

a. Solution Preparation

40 mM Citrate buffer having pH 3.1: 6.41 g of citric acid, anhydrous, and 1.96 g of Na Citrate were dissolved in 1 L of double distilled water (ddH₂O). The buffer solution was filtered by 0.2 μm filter (Nalgene cup filter).

800 mL of 10% HA solution having pH 5 was prepared by mixing 400 mL of buffer solution with 400 mL of 20% Grifols HA.

150 mg of Compound 1 was dissolved in 925 μL of formic acid to prepare a 150 mg/mL of Compound 1 solution.

b. Preparation of HA-Solubilized Compound 1 Solution

10% HA solution was precooled at 4° C. for 30 min. 800 mL of 10% HA solution was transferred into a 1000 mL beaker. The beaker was placed inside the water bath at 5° C. The solution was stirred at 5,000 rpm carefully to avoid formation of bubbles. The mixing blade was slowly raised to about 1 cm above the bottom of the beaker until the surface of the solution was circulating and turning over, again, being careful to avoid formation of bubbles. Compound 1 solution (533 μL for Formulation 7 and 266 μL for Formulation 8) was transferred drop-by-drop using a pipet, into the beaker while mixing, assuring no film formed on the top of the liquid surface. The mixing was continued for 5 minutes at 6,000 rpm. The mixer was stopped and the solution was kept at 5° C. for an additional 10 min.

c. Preparation of the Final Filtered Suspension

Two Nalgene cup 0.2 μm filters were prepared as follows: 10 mL from the bulk solution was taken using a pipette, and uniformly sprayed on the membrane to start the filtration process to saturate the membrane. The membrane was attached to a 1000 mL bottle and the remaining solution was filtered. The filtered suspension at was stored at 5° C. The bulk unfiltered suspension and final filtered suspension were assayed for Compound 1 content.

d. Lyophilization of Final Filtered Suspension

The lyophilizer was programmed to the cycle outlined in Table 20 above. A thin layer of vacuum grease was applied on the door seal if necessary and vacuum pump oil was replaced if necessary. Forty-eight 50 cc vials were filled with a 12 mL of final filtered suspension that resulted in 1.2 mg of Compound 1 per vial for Formulation 7 and 0.6 mg of Compound 1 per vial for Formulation 8. Stoppers were placed on the vials so that the vials were vented. The vials were loaded into the top shelf of the lyophilizer. The lyophilizer door was closed ensuring that a proper vacuum seal was formed. The lyophilzation process was started. After completion of the lyophilization cycle, the chamber was vented with dry nitrogen, and the vials were sealed before opening the door. The vials were removed, labeled and stored at room temperature.

e. Compound 1 Assay in the Lyophilized Sample

The following equipment was used to assay Compound 1 content in the lyophilized sample:

HPLC: Agilent Technologies 1260 Series with:
G7129A Vialsampler
G7111B Quat Pump
G7116A MCT
G7165A MWD

The following materials were used to assay Compound 1 content in the lyophilized sample:
Compound 1 reference standard
Compound 1 related impurities reference standards
Compound 1 lyophilized drug product formulated with HA, 1.2 mg in 50 cc vial
Water for injection, USP
Perchloric acid
Acetonitrile
0.2 μm syringe filter with Supor membrane, Pall
3 mL luer-lok syringe

The lyophilized products were reconstituted and prepared for the assay as follows:
The lyophilized drug product in the vial was reconstituted by carefully pipetting 22.8 mL of WFI into the side of the vial wall.
The lyophilized cake was allowed to fully reconstitute and dissolve for 30 minutes with periodic gentle swirling.
The sample was filtered through 0.2 μm filter using 3 mL luer-lok syringe. The first 0.5 mL was discarded and the remaining 1-2 mL of filtrate was collected.
1 mL of the sample filtrate was added into a 4 mL glass vial.
3 mL of acetonitrile was then added to the mixture to precipitate human serum albumin.
The mixture was incubated at room temperature for 10 minutes and 4° C. for 40 minutes.
0.75 mL of the mixture was pipetted and mixed with 0.75 mL of 0.05% perchloric acid in an HPLC vial. The mixture was gently mixed by vortexing.
The sample was assayed.

f. Diluent Preparation

The diluent containing 0.05% perchloric acid/acetonitrile in 70:30 ratio, prepared as follows was used in the assay: To a 1000 mL volumetric flask containing about 500 ml of water, 0.5 mL of perchloric acid was added, and diluted to volume with water. The contents were mixed well to obtain perchloric acid solution. 700 mL of perchloric acid solution and 300 ml of acetonitrile were added to a bottle and mixed well.

g. Stock Reference Standard Preparation

Compound 1 stock reference standard standard solution (500 μg/mL) was prepared as follows:

50 mg of Compound 1 reference standard was weighed into a 100 mL volumetric flask. 80 ml of acetonitrile was added to flask and sonicated until material was completely dissolved. Flask was allowed to equilibrate to room temperature. Contents were diluted to volume with acetonitrile and mix well.

h. Reference Standard Preparation

Compound 1 reference standard solution (12.5 μg/mL) was prepared as follows: 2.5 mL of Compound 1 stock reference standard (500 μg/mL) was pipetted into a 100 mL volumetric flask, diluted to volume with diluent and mixed well by vigorously shaking.

i. Analytical Method

The following analytical method was used:
Column: Waters ACQUITY UPLC@BEH C18 1.7 μm, 3.0×50 mm Column
Mobile Phase A: 0.1% TFA in 95:5 Water/MeCN
Mobile Phase B: 0.1% TFA in 5:95 Water/MeCN
Flow Rate: 0.6 mL/min
Column Temp (° C.): 35° C.
UV Detection: 235 nm
Injection Volume: 30 μL
Run Time: 13.5 minutes

TABLE 28
Gradient Setting

| Time (minutes) | % Mobile Phase A | % Mobile Phase B |
|---|---|---|
| 0 | 80 | 20 |
| 7 | 50 | 50 |
| 10.5 | 20 | 80 |
| 11.5 | 20 | 80 |
| 11.6 | 80 | 20 |
| 13.5 | 80 | 20 |

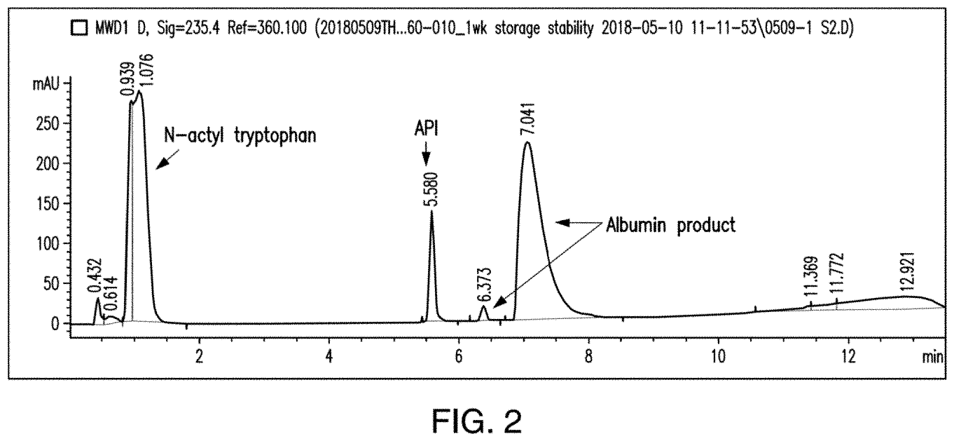

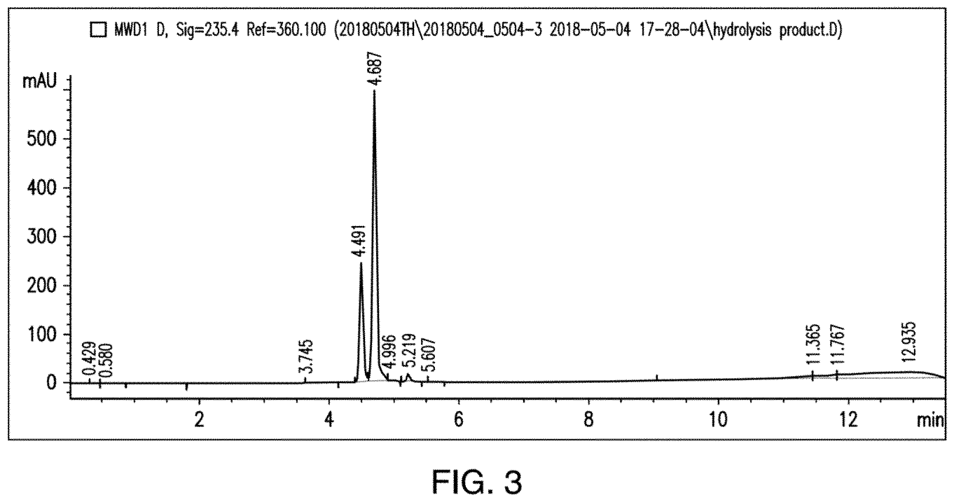

j. Related Impurities Reference Standard: The Related Impurities Reference Standard was Prepared by Dissolving 1 mg of Ring-Opened Standard into 1 mL of Water/MeCN 1:1 Co-Solvent. FIGS. 2 and 3 Provide Typical Chromatograms of Compound 1 and Related Impurities.

The Following Analytical Injection Sequence was Used:

TABLE 29
Injection sequence

| No. | Solution Name | Number of injections |
|---|---|---|
| 1 | Standard #1 | 1 |
| 2 | Sample 1 | 1 |
| 3 | Sample 2 | 1 |
| 4 | Sample 3 | 1 |
| 5 | Sample 4 | 1 |
| 6 | Sample 5 | 1 |
| 7 | Sample 6 | 1 |
| 8 | Standard #2 | 1 |

k. Standard Curve Generation:

The API concentration was calculated comparing to the 12.5 μg/mL standard

API (Conc)=PAₛₐₘₚ/PA_{std}×Conc_{std}×8

II. Formulation with 10% Human Albumin and 6.7% Sucrose: Formulation 9 (HA/Compound 1 (1000:1))

a. Solution Preparation
40 mM Citrate buffer having pH 3.1: 6.41 g of citric acid, anhydrous, and 1.96 g of Na Citrate were dissolved in 1 L of double distilled water (ddH₂O). The buffer solution was filtered by 0.2 μm filter (Nalgene cup filter).
400 mL of 20% HA solution was transferred into 500 mL of media storage bottle.
107.2 g of sucrose was weighed and added into the HA solution. The bottle was gently swirled to completely dissolve the sucrose.
The sugar HA solution was transferred into a 1000-mL glass cylinder.
40 mM Citrate buffer was added to make 800 mL 10% HA solution having pH 5.0.
150 mg of Compound 1 was weighed and dissolved in 925 μL of formic acid to prepare a 150 mg/mL of Compound 1 solution.

b. Preparation of HA-Solubilized Compound 1 Solution

10% HA solution was precooled at 4° C. for 30 min. 800 mL of 10% HA solution was transferred into a 1000 mL beaker. The beaker was placed inside the water bath at 5° C. The solution was stirred at 5,000 rpm carefully to avoid formation of bubbles. The mixing blade was slowly raised to about 1 cm above the bottom of the beaker until the surface of the solution was circulating and turning over, again, being careful to avoid formation of bubbles. Compound 1 solution (533 μL) was transferred drop-by-drop using a pipet, into the beaker while mixing, assuring no film formed on the top of the liquid surface. The mixing was continued for 5 minutes at 6,000 rpm. The mixer was stopped and the solution was kept at 5° C. for an additional 10 min.

c. Preparation of the Final Filtered Suspension

The final filtered suspension was prepared as described in Example 3, I.

d. Lyophilization of Final Filtered Suspension

The lyophilizer was programmed to the cycle outlined in Table 23. A thin layer of vacuum grease was applied on the door seal if necessary and vacuum pump oil was replaced if necessary. Fifty 50 cc vials were filled with a 24 mL of final filtered suspension that resulted in 1.2 mg of Compound 1 per vial. Stoppers were placed on the vials so that the vials were vented. The vials were loaded into the top shelf of the lyophilizer. The lyophilizer door was closed ensuring that a proper vacuum seal was formed. The lyophilzation process was started. After completion of the lyophilization cycle, the chamber was vented with dry nitrogen, and the vials were sealed before opening the door. The vials were removed, labeled and stored at room temperature.

e. Reconstitution of Lyophilized Product

The lyophilized products were reconstituted and prepared for the assay as follows:
The lyophilized drug product in the vial was reconstituted by carefully pipetting 22 mL of WFI into the side of the vial wall.
The lyophilized cake was allowed to fully reconstitute and dissolve for 30 minutes with periodic gentle swirling.
The sample was filtered through 0.2 μm filter using 3 mL luer-lok syringe. The first 0.5 mL was discarded and the remaining 1-2 mL of filtrate was collected.
1 mL of the sample filtrate was added into a 20 mL glass vial.
1 mL of 0.05% perchloric acid was added into the vial to dilute the sample solution.
3 mL of acetonitrile was then added to the mixture to precipitate human serum albumin.
The mixture was incubated overnight at 4° C.
3 mL of 0.05% perchloric acid was added to the cold solution to make a final 8 mL solution.
1 mL of the supernatant was then taken from the 8 mL solution and transferred to the HPLC vial
The sample was assayed.

The diluent containing 0.05% perchloric acid/acetonitrile in 70:30 ratio was prepared as described in Example 3, I.

Compound 1 stock reference standard, Compound 1 reference standard solution and related impurities reference standard were prepared as described in Example 3, I.

The analytical method described in Example 3, I was used for the assay.

III. Formulation with 5% Human Albumin and 8% Sucrose: Formulation 10 (HA/Compound 1 (1000:1))

a. Solution Preparation

30 mM Citrate buffer having pH 4.2: 6.64 g of citric acid, anhydrous, and 7.5 g of Na Citrate were dissolved in 2 L of double distilled water (ddH₂O). The buffer solution was filtered by 0.2 μm filter (Nalgene cup filter).
350 mL of 20% HA solution was transferred into 500 mL of media storage bottle.
112 g of sucrose was weighed and added into the HA solution. The bottle was gently swirled to completely dissolve the sucrose.
The sugar HA solution was transferred into a 2000-mL glass cylinder.
30 mM Citrate buffer was added to make 1.4 L 5% HA solution having pH 5.0.
150 mg of Compound 1 was weighed and dissolved in 925 μL of formic acid to prepare a 150 mg/mL of Compound 1 solution.

b. Preparation of HA-Solubilized Compound 1 Solution

5% HA solution was precooled at 4° C. for 30 min. 700 mL of 5% HA solution was transferred into a 1000 mL beaker. The beaker was placed inside the water bath at 5° C. The solution was stirred at 5,000 rpm carefully to avoid formation of bubbles. The mixing blade was slowly raised to about 1 cm above the bottom of the beaker until the surface of the solution was circulating and turning over, again, being careful to avoid formation of bubbles. Compound 1 solution (467 μL) was transferred drop-by-drop using a pipet, into the beaker while mixing, assuring no film formed on the top of the liquid surface. Mixing was continued for 5 minutes at 6,000 rpm. The mixer was stopped and the solution was kept at 5° C. for an additional 10 min. Another 700 mL of Formulation 10 was prepared repeating these steps.

c. Preparation of the Final Filtered Suspension

The final filtered suspension was prepared as described in Example 3, I.

d. Lyophilization/Reconstitution

The samples were lyophilized as described in Example 3, II. The lyophilized samples were assayed for Compound 1 using the equipment and materials described in Example 3, I.

The lyophilized products were reconstituted and prepared for the assay as described in Example 3, II.

The diluent containing 0.05% perchloric acid/acetonitrile in 70:30 ratio was prepared as described in Example 3, I.

Compound 1 stock reference standard, Compound 1 reference standard solution and related impurities reference standard were prepared as described in Example 3, I.

The analytical method described in Example 3, I was used for the assay.

IV. Formulation with 10% Recombinant Human Serum Albumin (rHSA): Formulation 11 (HA/Compound 1 (500:1))

a. Solution Preparation

0.96 g of citric acid, anhydrous, and 0.29 g of Na Citrate were dissolved in 300 mL of 10% rHSA.
150 mg of Compound 1 was weighed and dissolved in 925 μL of formic acid to prepare a 150 mg/mL of Compound 1 solution.

b. Preparation of HA-Solubilized Compound 1 Solution

10% HA solution was precooled at 4° C. for 30 min. 300 mL of 10% HA solution was transferred into a 500 mL beaker. The beaker was placed inside the water bath at 5° C. The solution was stirred at 5,000 rpm carefully to avoid formation of bubbles. The mixing blade was slowly raised to about 1 cm above the bottom of the beaker until the surface of the solution was circulating and turning over, again, being careful to avoid formation of bubbles. Compound 1 solution (400 μL) was transferred drop-by-drop using a pipet, into the beaker while mixing, assuring no film formed on the top of the liquid surface. Mixing was continued for 5 minutes at 6,000 rpm. The mixer was stopped and the solution was kept at 5° C. for an additional 10 min.

c. Preparation of the Final Filtered Suspension

The final filtered suspension was prepared as described in Example 3, I.

d. Lyophilization of Final Filtered Suspension

The lyophilizer was programmed to the cycle outlined in Table 22. A thin layer of vacuum grease was applied on the door seal if necessary and vacuum pump oil was replaced if necessary. Thirty five 20 cc vials were filled with 6 mL of final filtered suspension that resulted in 1.2 mg of Compound 1 per vial. Stoppers were placed on the vials so that the vials were vented. The vials were loaded into the top shelf of the lyophilizer. The lyophilizer door was closed ensuring that a proper vacuum seal was formed. The lyophilzation process was started. After completion of the lyophilization cycle, the chamber was vented with dry nitrogen, and the vials were sealed before opening the door. The vials were removed, labeled and stored at room temperature.

e. Reconstitution

The lyophilized products were reconstituted and prepared for the assay as described in Example 3, I.

The diluent containing 0.05% perchloric acid/acetonitrile in 70:30 ratio was prepared as described in Example 3, I.

Compound 1 stock reference standard, Compound 1 reference standard solution and related impurities reference standard were prepared as described in Example 3, I.

The analytical method described in Example 3, I was used for the assay.

V. Formulation with 10% Human Albumin: Formulation 12 (HA/Compound 1 (500:1))

a. Solution Preparation

40 mM Citrate buffer having pH 3.1 was prepared by dissolving 6.64 g of citric acid, anhydrous, and 7.5 g of Na Citrate in 2 L of double distilled water (ddH₂O).
200 mL of buffer solution was mixed with 200 mL of 20% HA solution to generate 400 mL of 10% HA solution having pH 5.0.
150 mg of Compound 1 was weighed and dissolved in 925 μL of formic acid to prepare a 150 mg/mL of Compound 1 solution.

b. Preparation of HA-Solubilized Compound 1 Solution

10% HA solution was precooled at 4° C. for 30 min. 400 mL of 10% HA solution was transferred into a 500 mL beaker. The beaker was placed inside the water bath at 5° C. The solution was stirred at 5,000 rpm carefully to avoid formation of bubbles. The mixing blade was slowly raised to about 1 cm above the bottom of the beaker until the surface of the solution was circulating and turning over, again, being careful to avoid formation of bubbles. Compound 1 solution (533 μL) was transferred drop-by-drop using a pipet, into the beaker while mixing, assuring no film formed on the top of the liquid surface. The mixing was continued for 5 minutes at 6,000 rpm. The mixer was stopped and the solution was kept at 5° C. for an additional 10 min.

c. Preparation of the Final Filtered Suspension

The final filtered suspension was prepared as described in Example 3, I.

d. Lyophilization/Reconstitution

The samples were lyophilized as described in Example 3, IV. The lyophilized samples were assayed for Compound 1 using the equipment and materials described in Example 3, I.

The lyophilized products were reconstituted and prepared for the assay as described in Example 3, I.

The diluent containing 0.05% perchloric acid/acetonitrile in 70:30 ratio was prepared as described in Example 3, I.

Compound 1 stock reference standard, Compound 1 reference standard solution and related impurities reference standard were prepared as described in Example 3, I.

The analytical method described in Example 3, I was used for the assay.

### Example 4: Preparation of 5 L Batch of Formulation

A 5 L batch of a formulation having composition shown in Table 15 (Formulation 15) was prepared using the materials and procedure described below.

### Materials

The following materials were used in preparation of the formulation:
Compound 1 (250 mg in formulation, 300 mg total required)
Ring opened Compound 1 related impurity reference standard
Albumin (Human), 20 g, 100 mL, Grifols, Lot No.IBAC5D8001 (250 g, 12.5 vials in formulation, 13 vials required)
MilliQ water, 18.4 MOhms-cm and 4 ppb TOC
Hyclone Hypure endotoxin-free cell culture grade water, GE Life Sciences, Cat. # SH3052903
Formic acid 97%, Alfa Aesar, A13285
Sodium citrate, dihydrate, BDH, 8017-500G
Citric acid, anhydrous, Spectrum, CI133
Sterile 70% Isopropanol, VWR, Cat. #89108-162
5 mL conical bottom V-vials with PTFE-lined screw cap, Wheaton, Cat. # W986299NG
Nalgene Rapid-Flow sterile disposable bottle top filters with 0.2 μm PES membrane, Thermo Fisher Scientific, #295-3345
Sterile 1000 mL Nalgene bottles
Acrodisc 13 mm syringe filters with 0.2-μm Supor membrane, Pall Life Sciences, Part #4602
Sterile glass vials, 50 cc-20 mm, Allergy Laboratories, Inc.
Sterile FluroTec coated 20 mm stoppers
PETG media storage bottles
Nitrogen.

Equipment

The following equipment was used to prepare the formulation:
Overhead impeller mixer
Bath sonicator
Biosafety cabinet
Lyophilizer
pH meter

### Formulation

Table 30 below provides composition of compounding solutions:

TABLE 30
Composition of compounding solutions

| | | | Density at |
|---|---|---|---|
| | Component | Weight | 20° C. (est.) |
| 30 mM Citrate | Citric acid, anhydrous | 11.91 g | — |
| Buffer | Sodium citrate, dihydrate | 13.47 g | — |
| | Water for Injection | 3,579 g | 0.9982 g/mL |
| HA and sucrose | 20% HA | 1,321 g | 1.057 g/mL |
| solution | Sucrose | 300.0 g | — |
| | 30 mM citrate buffer | 3,604 g | 1.003 g/mL |
| Organic solution | Compound 1 | 300.0 mg | — |
| of Compound 1 | Formic acid | 2.257 g | 1.221 g/mL |
| Final | Organic solution of | 2.137 g | 1.282 g/mL |
| compounded | Compound 1 | | |
| bulk solution | HA and sucrose solution | 5,225 g | 1.045 g/mL |

Table 31 below provides final bulk formulation composition.

TABLE 31
Bulk formulation composition

| Component | Weight |
|---|---|
| Citric acid, anhydrous | 11.91 g |
| Sodium citrate, dihydrate | 13.47 g |
| Water for Injection | 3,579 g |
| 20% HA | 1,321 g |
| Sucrose | 300.0 g |
| Compound 1 | 250.0 mg |
| Formic acid | 1.887 g |

Table 32 below provides concentrations of constituents in final bulk solution.

TABLE 32
Concentrations of constituents in final bulk solution

| Component | Concentration |
|---|---|
| Compound 1 | 50 μg/mL |
| HA | 50 mg/mL |
| Sucrose | 60 mg/mL |
| Sodium chloride | 22.5 mg/mL |
| Citrate | 4.5 mg/mL |
| Formic acid | 377 μg/mL |

### Equipment Preparation

The mixer was cleaned by rinsing twice with water, followed by a rinse with 70% IPA, followed by a final rinse with MilliQ water of WFI. The shelves and chambers of the lyophilizer were wiped with 70% IPA. The nitrogen gas cylinder was connected to the vacuum release inlet port of the lyophilizer with an in-line 0.2 μm sterilizing filter and the regulator was set to 5 mbar.

Solution Preparation

3,604 g of 30 mM Citrate buffer having pH 4.2 was prepared by dissolving 11.91 g of citric acid, anhydrous, and 13.47 g of Na Citrate in 3,579 g of water for injection (or equivalent). The mixture was mixed with the overhead impeller mixer until solids were well dissolved.
1,250 mL (1,321 g) of 20% HA solution was carefully transferred into the media storage carboy so as not to generate foam.
300.0 g of sucrose was added to the HA solution.
The mixer head was placed into the solution so that it sat just above the bottom of the carboy. The mixer was started at the lowest speed, being careful to not entrain air and form bubbles. Without entraining bubbles, the solution was gently mixed to completely dissolve the sucrose and until the solution was homogeneous.
The pH was maintained between 4.8 and 5.2,
300 mg of Compound 1 was weighed and dissolve in 1,850 μL of formic acid to prepare a 150 mg/mL solution of Compound 1. The solution was sonicated in a warm water bath to completely dissolve the compound.

Preparation of HA-Solubilized Compound 1 Solution

The mixer head was placed into the 5% HA and 6% sucrose solution so that it sat just above the bottom of the carboy. The mixer was started at the lowest speed, being careful to not entrain air and form bubbles. The mixer speed was increased until the surface of the solution was circulating and turning over, again, being careful to avoid entrainment of air and formation of bubbles.
1,667 μL of Compound 1 solvent solution was pipetted dropwise at a rate of approximately 50 μL (approximately one drop) every 10 seconds into the carboy while mixing, assuring no film formed on the top of the liquid surface.
The mixing was continued for an additional 10 minutes.
The bulk unfiltered suspension was sampled and frozen for assay of Compound 1 content and stability.
The unfiltered suspension was stored at 5° C. until ready for filtration.

Preparation of the Final Filtered Suspension

In the biosafety cabinet, two 0.2 μm Nalgene cup filters were prepared by connecting to vacuum.
10 mL of the mixed bulk solution was pipetted and uniformly spread on one of the filter membranes. The liquid was pulled through the membrane with vacuum to saturate, the receiving flask was removed and contents of the flask were disposed.
The same cup filter was attached with the saturated membrane to a new sterile 1000 mL Nalgene receiving bottle, and 1 L of the solution was filtered. The receiving bottle was removed and capped.
A new sterile 1000 mL Nalgene receiving bottle was attached and another 1 L of solution was filtered. The steps were repeated until all 5 L of solution was filtered.
The final filtered suspension was sampled for pH and density measurements.
The filtered suspension was stored at 5° C. until ready for vial filling.

Lyophilization of Final Filtered Solution

The lyophilizer was programmed to the cycle outlined in Table 24. A thin layer of vacuum grease was applied on the door seal if necessary. The vacuum pump oil was replaced if necessary.
Approximately 250-50 cc vials were filled with 20 mL of the final filtered suspension to result in 1.0 mg of Compound 1 per vial.
Stoppers were placed on vials so that the vials were vented and loaded into the top shelf of the lyophilizer.
The lyophilizer door was closed and a proper vacuum seal formation was ensured.
The lyophilzation process was started within 48 hours of bulk solution preparation, where the solution may be held at room temperature for no more than 24 hours during that period.
When the lyophilization cycle was complete, the chamber was vented with dry nitrogen and the vials were sealed at ˜500 torr pressure before opening the door.
The vials were removed, labeled and stored at room temperature.

Compound 1 and Related Impurities Assay in the Lyophilized Sample

The following equipment was used to assay Compound 1 content in the lyophilized sample:
Agilent Technologies 1260 Series HPLC with:
G7129A Vial sampler
G7111B Quaternary pump
G7116A Multi-column thermostat
G7165A Multi-wavelength detector

The following materials were used to assay Compound 1 content in the lyophilized sample:
Compound 1 reference standard
Compound 1 related impurities reference standards
Compound 1 lyophilized drug product formulated with HA, 1.0 mg in 50 cc vial Water for injection, USP
Perchloric acid
Acetonitrile
Trifluoroacetic acid
0.2 μm syringe filter with Supor membrane, Pall
3 mL luer-lok syringe

Standards and Diluent Preparation

The diluent containing 0.05% perchloric acid/acetonitrile in 70:30 ratio, prepared as follows was used in the assay: To a 1000 mL volumetric flask containing about 500 ml of water, 0.5 mL of perchloric acid was added, and diluted to volume with water. The contents were mixed well to obtain perchloric acid solution. 700 mL of perchloric acid solution and 300 ml of acetonitrile were added to a bottle and mixed well.

Compound 1 stock reference standard standard solution (500 μg/mL) was prepared as follows:

50 mg of Compound 1 reference standard was weighed into a 100 mL volumetric flask. 80 ml of acetonitrile was added to flask and sonicated until material was completely dissolved. Flask was allowed to equilibrate to room temperature. Contents were diluted to volume with acetonitrile and mix well.

Compound 1 reference standard solution (12.5 μg/mL) was prepared as follows: 2.5 mL of Compound 1 stock reference standard (500 μg/mL) was pipetted into a 100 mL volumetric flask, diluted to volume with diluent and mixed well by vigorously shaking.

Related impurities reference standard was prepared by dissolving 1 mg of ring-opened Compound 1 standard into 1 mL of water/acetonitrile 1:1 cosolvent.

Sample Preparation

The lyophilized products were reconstituted and prepared for the assay as follows:
The lyophilized drug product in the vial was reconstituted by carefully pipetting 18.6 mL of WFI into the side of the vial wall.
The lyophilized cake was allowed to fully reconstitute and dissolve for 30 minutes with periodic gentle swirling.
The sample was filtered through 0.2 μm filter using 3 mL luer-lok syringe. The first 0.5 mL was discarded and the remaining 1-2 mL of filtrate was collected as a sample.
1 mL of the unfiltered or filtered sample was added into a 20 mL glass vial.
1 mL of 0.05% perchloric acid was added into the vial to dilute the sample solution.
3 mL of acetonitrile was then added to the mixture to precipitate human serum albumin.
The mixture was incubated at 4° C. overnight.
3 mL of 0.05% perchloric acid was added to the cold solution to make a final 8 mL solution.
1 mL of the supernatant from the 8 mL solution was transferred to an HPLC vial.

Analytical Method

The following analytical method was used:
Column: Waters ACQUITY UPLC@BEH C18 1.7 μm, 3.0×50 mm Column
Mobile Phase A: 0.1% TFA in 95:5 Water/MeCN
Mobile Phase B: 0.1% TFA in 5:95 Water/MeCN
Flow Rate: 0.6 mL/min
Column Temp (° C.): 35° C.
UV Detection: 235 nm
Injection Volume: 30 μL
Run Time: 13.5 minutes
Injection sequence: 12.5 g/mL Compound 1 reference standard, followed by 6 samples, followed by the 12.5 g/mL Compound 1 reference standard

TABLE 33
Gradient Setting

| Time (minutes) | % Mobile Phase A | % Mobile Phase B |
|---|---|---|
| 0 | 80 | 20 |
| 7 | 50 | 50 |
| 10.5 | 20 | 80 |
| 11.5 | 20 | 80 |
| 11.6 | 80 | 20 |
| 13.5 | 80 | 20 |

Data Analysis

The peak area of API (PAₛₐₘₚ) was calculated using peak eluting at approximately 5.58 min. The peak area of related impurities was calculated using peaks eluting at approximately 4.49 min and 4.69 min.

The API concentration was calculated by comparing to the 12.5 μg/mL standard using the formula:

Conc_{API}=PAₛₐₘₚ/PA_{std}×Conc_{std}×8.

The related impurity content was calculated with respect to the API peak area.

Human Albumin Assay

The same equipment as that used to assay Compound 1, described above, was used for human albumin assay.

Material:

The following material was used human albumin assay:
Albumin (Human), 20 g 100 mL, Grifols
Compound 1 lyophilized drug product formulated with human albumin, 1.0 mg/50 cc vial
Potassium phosphate dibasic (K₂HPO₄), anhydrous USP
Concentrated hydrochloric acid, ACS reagent grade
Water for injection, USP
0.2 m syringe filter with Supor membrane, Pall

Standards and Diluent Preparation

A 0.10 M K₂HPO₄ mobile phase was prepared by dissolving 34.84 g of potassium phosphate dibasic powder in 1500 mL of water. The pH was adjusted to pH 7.0+/−0.1 with 1M hydrochloric acid. The solution was transferred to 2 L volumetric flask and QS to the mark with water. A 1 mg/mL human albumin standard was prepared by transferring 0.5 mL of HA solution into 100 mL volumetric flask and QS to the mark with saline.

Sample Preparation

The lyophilized content of a drug product vial was reconstituted by carefully pipetting 18.6 mL of WFI into the side of the vial wall. The lyophilized cake was allowed to fully reconstitute and dissolve for 30 minutes with periodic gentle swirling. The reconstituted sample was diluted to ˜1 mg/mL HA by adding 200 μL of sample to 10 mL volumetric flask QS to mark with saline. 1 mL of the HA standard solution was transferred to a HPLC vial.

Analytical Method
Column: TOSOH Bioscience, LLC TSKgel G300SW 7.8 mm ID×30 cm, 5 μm column # S7363-06R
Mobile Phase: 0.10 M K₂HPO₄
Flow Rate: 1.0 mL/min
Column Temp: Ambient
UV Detection: 228 nm
Injection Volume: 10 μL
Needle Wash: Water
Run Time: 30 minutes
Method blank: 60 min

Formic Acid Assay

HPLC procedure used for determination of residual DMSO and formic acid in Compound 1 drug product was as described below.

Equipment
Microbalance or semi-micro analytical balance
HPLC system
HPLC software for data acquisition and data process
Class A volumetric flasks
Class A graduated cylinders and pipettes or autopipettes
Sonicator

Materials and Reagents
Deionized Water
Dimethyl Sulfoxide
Formic Acid
Potassium Phosphate Monobasic

HPLC Conditions
Column: Grace Prevail Organic Acid 3 jam, 150 mm×4.6 mm, P/N 88655 or equivalent
Column temperature: Ambient
Detector: UV @ 210 nm
Mobile Phase: 25 mM KH₂PO₄, pH=3.25
Flow rate: 1.0 ml/min
Run time: 10 minutes
Injection volume: 10 μL

Notes on Column Washing/Conditioning:

i. At the end of the analytical sequence, column was washed with 80:20 (acetonitrile/water).

ii. Before starting sequence, column was thoroughly conditioned with mobile phase so that peak retention is impacted negatively.

Diluent
Mobile Phase
Preparation of Mobile Phase
Approximately 6.8 g of potassium phosphate monobasic was weighed and dissolved in 2 liters of deionized water, pH was adjusted to 3.25 with phosphoric acid.

Preparation of Standard Solutions
Preparation of Stock Standard Solution: 1% v/v
In a 200 mL volumetric flask containing about 100 mL of diluent, 2 ml of DMSO and 2 ml of formic acid were pipetted using a glass pipette. The contents were diluted to volume with diluent and mixed well.
Preparation of Working Standard Solution: 0.005% v/v
In a 200 mL volumetric flask, 1 ml of stock standard solution was pipetted using a glass pipette. The content was diluted to volume with diluent and mixed well.
Preparation of Quantitation Limit (QL) Solution: 0.0005% v/v
In a 50 ml volumetric flask, ˜25 mL of diluent and 5 mL of working standard solution (0.005% v/v) were added. The contents were diluted to volume with diluent and mixed well.

The standard solutions were stable for 6 days when stored under ambient conditions.

Preparation of Compound 1 drug product sample solution (n=6)
a) 6 vials were randomly selected from beginning, middle and end locations.
b) 10 mL of diluent was pipetted into each vial.
c) Contents were dissolved by shaking vigorously, the solution was transferred into a 100 mL volumetric flask.
d) The product vials were rinsed 3 times with diluent and all rinses were transferred into the 100 mL volumetric flask.
e) Contents were diluted to volume with diluent.

The sample solutions were stable for 3 days when stored under ambient conditions.

System Suitability
No sample solution was injected until the criteria below are met.

| Seq. Line | Sample |
|---|---|
| 1 | Blank Diluent |
| 2 | QL Solution |
| 3 | Working Standard Solution (6 injections) |

System Suitability Criteria
a) Injection of Blank Diluent has no significant interfering peak at the retention time of DMSO and formic acid.
b) The signal to noise ratio for DMSO and formic acid in QL must be not less than (NLT) 10.
c) % RSD of peak areas of six consecutive injections for DMSO and formic acid from the Working Standard Solution should be NMT 3%.

Analytical Procedure

Injections of standard and sample test solutions were made according to the following sequence and the chromatograms were recorded. Additional sample brackets were added as necessary.

TABLE 34

| Injection Number | Injection ID |
|---|---|
| 1 | Standard |
| 2 | Sample 1 |
| 3 | Sample 2 |
| 4 | Sample 3 |
| 5 | Sample 4 |
| 6 | Sample 5 |
| 7 | Sample 6 |
| 8 | Standard |

Calculations

$$( mg \text{/} vial ) Solvent = \frac{\begin{matrix}Sample Peak Area \times Std Concentration \times Sample Dilution ( ml ) \times \\ Density ( g \text{/} ml ) \times 1000 ( mg \text{/} g )\end{matrix}}{Standard Peak Area \times 100}$$

### Where:

i. Density of DMSO=1.1 g/ml

ii. Density of formic acid=1.22 g/ml

iii. Sample Dilution=100 ml

iv. Std Concentration (%)=0.005

### Example 5: Thermal Analysis of Lyophilization Formulations

In this study, a series of thermal analysis were conducted on the formulation described in Example 4 with freeze drying microscope (FDM) and differential scanning calorimetry (DSC) to determine the collapse temperature and the Tg′ of the formulation.

Differential Scanning Calorimetry (DSC) Analysis

The DSC analysis was conducted using the following two methods:

Method #1
Initial temperature: 20.00° C.
Equilibrate at 20.00° C.
Ramp 10° C./min to −60° C.
Equilibrate at −60° C. for 2 minutes
Mark end of cycle 1
Ramp 10° C./min to 20° C.
Equilibrate at 20.00° C.
Mark end of cycle 2
End of method

Method #2—Modulated
Initial temperature: 20.00° C.
Equilibrate at 20.00° C.
Ramp 2° C./min to −60° C.
Equilibrate at −60° C. for 2 minutes
Mark end of cycle 1
Modulate temp. 1° C./min for 60 seconds
Isothermal for 5 minutes
Mark end of cycle 2
Ramp 2° C./min to 20° C.

Table 35 below summarizes the DSC characterization results for the formulation of Example 4.

TABLE 35
DSC Results (Temperature ° C.)

| | Method 1/Run 1 | Method 2/Run 1 | Method 2 |
|---|---|---|---|
| | (Formulation of | (Formulation of | Run 2 |
| | Example 4) | Example 4) | (5% HA only) |
| Nucleation | −25.12 | −19.82 | −23.86 |
| Onset | | | |
| (Tnu) | | | |
| Glass | −28.39 | −31.12 | N/A |
| Transition | | | |
| (Tg′) | | | |
| Ice Melt | 1.01 | −1.50 | −0.10 |
| (Te) | | | |

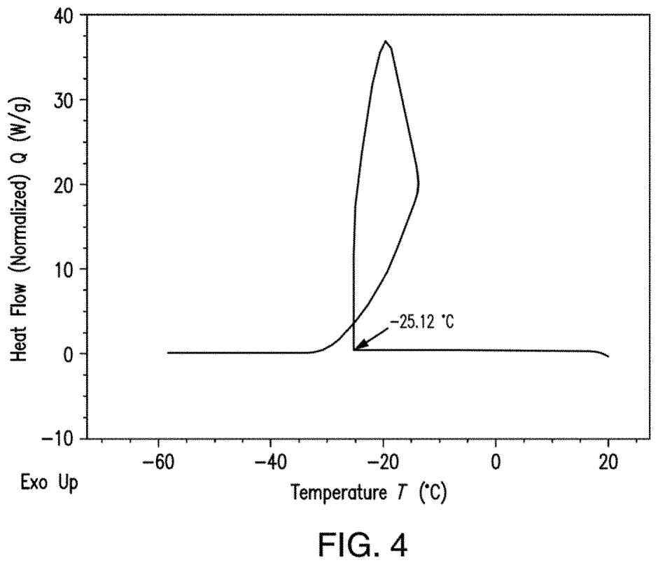

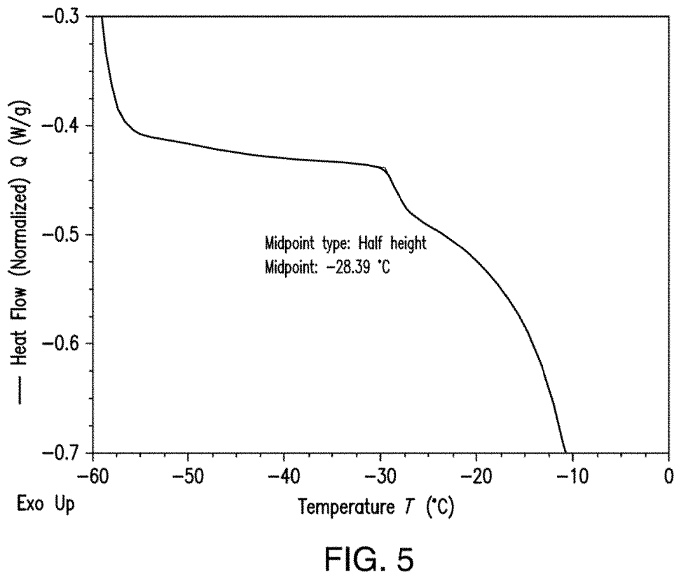

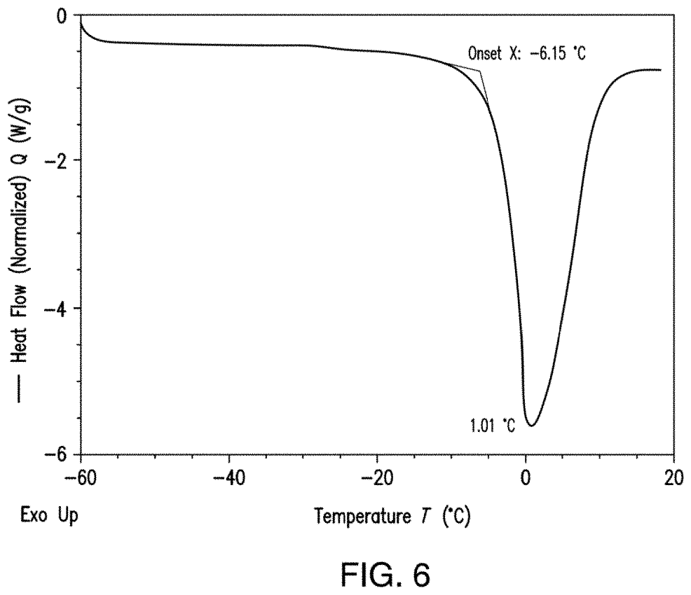

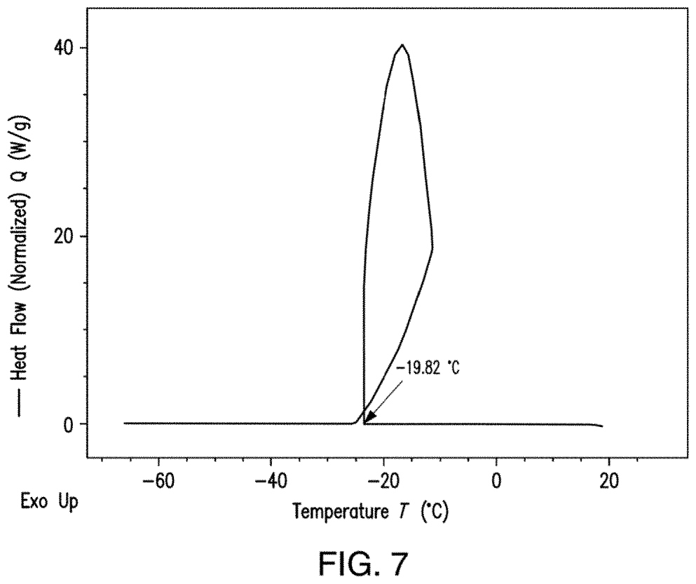

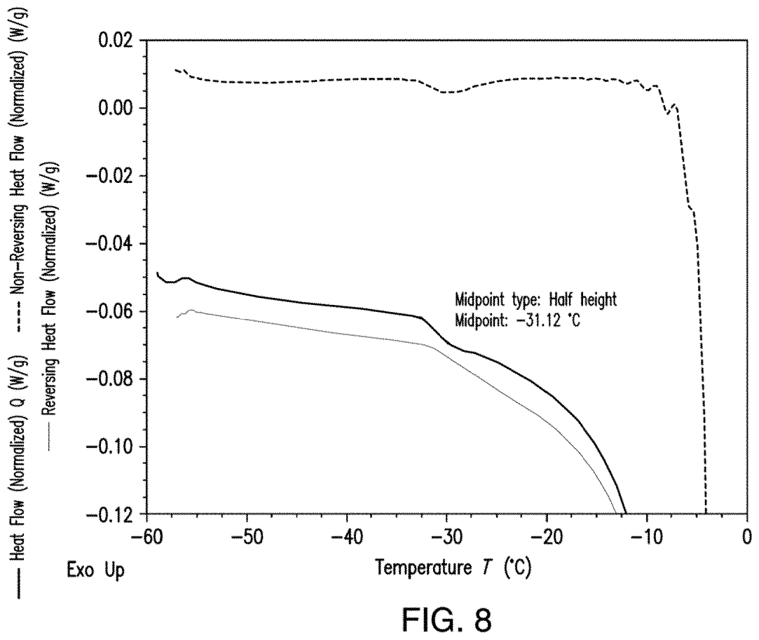

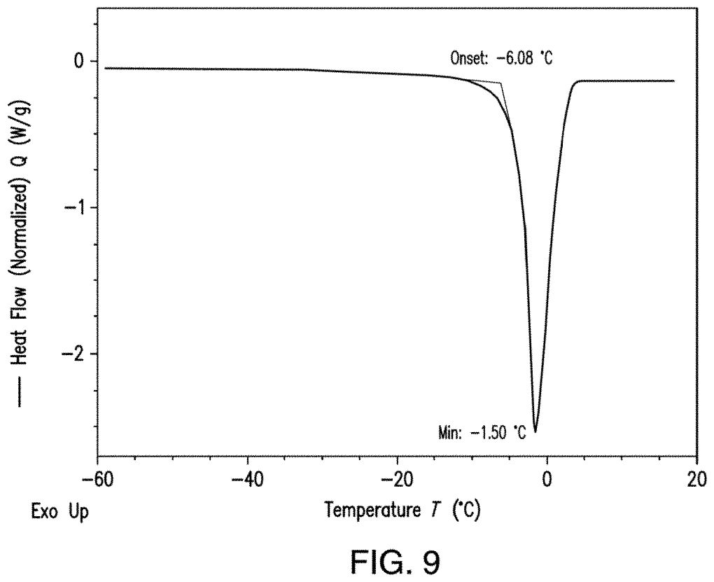

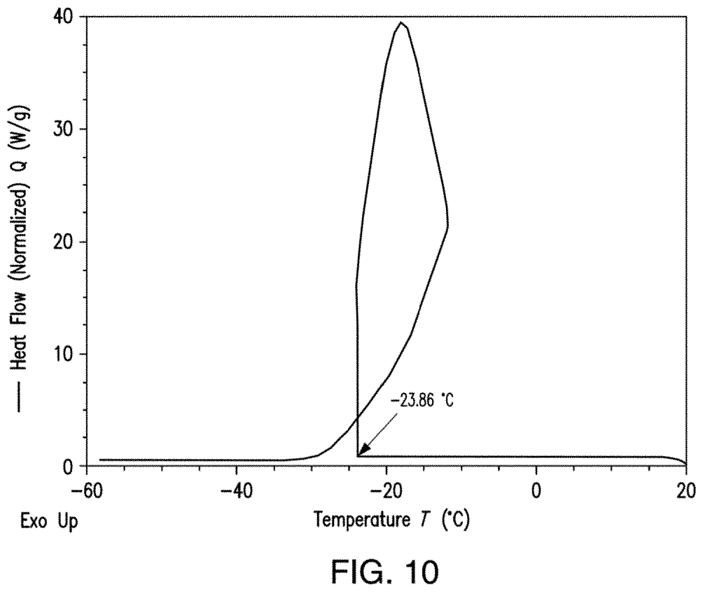

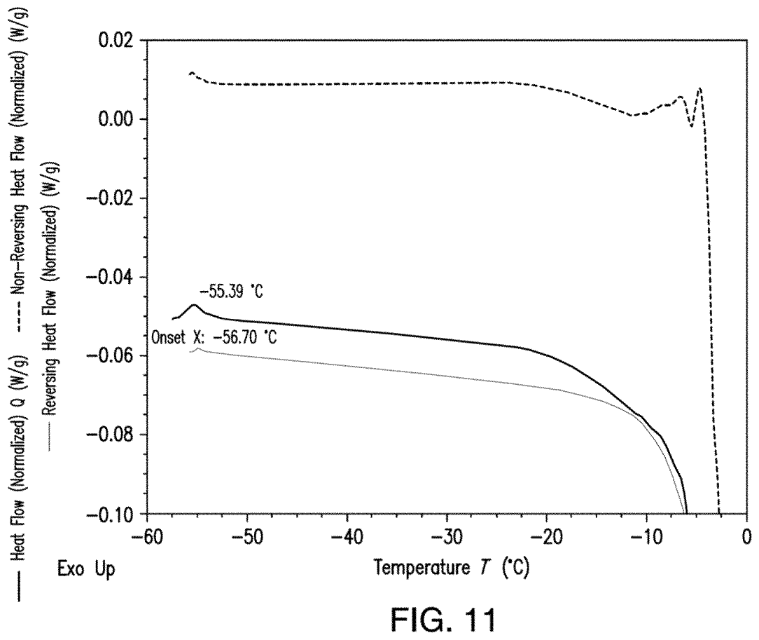

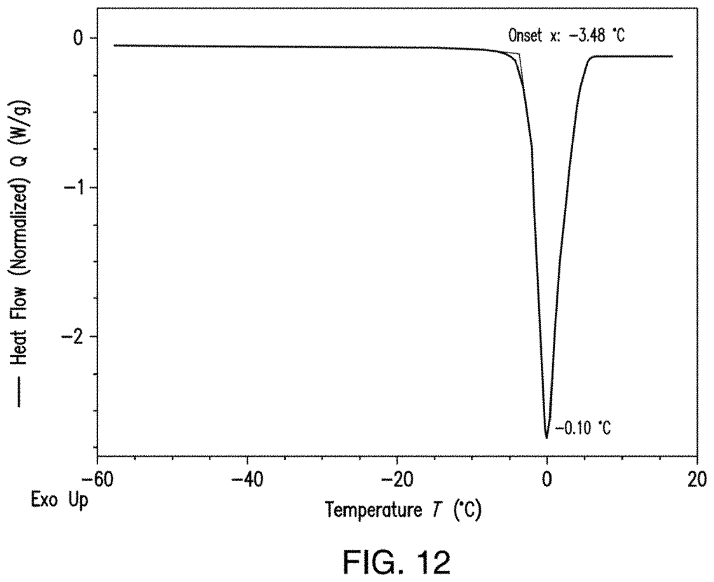

FIGS. 4, 5 and 6 provide plots showing nucleation onset temperature, glass transition temperature and ice melt temperature for method 1, run 1 and FIGS. 7, 8 and 9 provide plots showing nucleation onset temperature, glass transition temperature and ice melt temperature for method 2, run 1, for the formulation of Example 4. FIGS. 10, 11 and 12 provide plots showing nucleation onset temperature, melt curve and ice melt temperature for method 2, run 2, for 5% HSA.

Freeze Drying Microscopy (FDM) Analysis

The freeze drying microscope used in this study was Linkam Scientific Instruments FDCS 196 freeze drying microscope. FDCS 196 is designed to determine the temperature at which frozen material undergoes changes that may be critical to its freeze-drying behaviour. This unit contained a small freeze-drying chamber in which the freeze-drying response of a thin sample of product can be observed microscopically and recorded as a series of digital images.

Before starting the experiment, the stage chamber was prepared for vacuum by ensuring that it was clean and dry. Silicone oil was used to ensure good thermal transfer from the stage to the slide and sample. A quartz slide was loaded onto the stage and a small aliquot of pre-formulated liquid sample was placed on top of the slide and covered with a cover slip. A shim was used to ensure a uniform sample thickness, if needed.

The lyophilization characteristics were determined using FDCS 196 with the Linksys 32 image and data capture software. A pipette was used to dispense a 2-4 ml sample onto the slide. The sample was frozen and/or annealed and vacuum was applied to the system as indicated in the Tables 36 and 37 below. The sample was warmed and drying was observed through the digital camera connected to the microscope. The images were captured at various intervals throughout the process. Following the FDM cycle, the images were observed and the first signs of collapse/eutectic melt were noted and the corresponding temperature associated with that image was recorded.

TABLE 36
Freeze drying cycle
Method #1

| Step | Set Point |
|---|---|
| Load Temperature | 20° C. |
| Freeze Rate | 10° C./min |
| Freeze Temperature SP | −45° C. |
| Freeze Time | 10 min |
| Vacuum SP | 60 mT |
| Warm Rate | 1° C./min |
| Warm Temperature SP | 20° C. |

TABLE 37
Freeze drying cycle
Method #2 - Annealing

| Step | Set Point |
|---|---|
| Load Temperature | 20° C. |
| Freeze Rate | 10° C./min |
| Freeze Temperature SP | −45° C. |
| Freeze Hold Time | 10 min |
| Warm Rate | 3° C./min |
| Annealing Temperature SP | −15° C. |
| Annealing Time | 10 min |
| Freeze Rate | 3° C./min |
| Freeze Temperature SP | −45° C. |
| Freeze Time | 10 min |
| Vacuum SP | 50 mT |
| Warm Rate | 1° C./min |
| Warm Temperature SP | 20° C. |

The collapse zone/eutectic melt behavior in freeze-dry microscopy analysis are shown in Table 38 below.

TABLE 38
Summary of critical temperatures observed

| | | Collapse | Total Collapse |
|---|---|---|---|
| Method/sublot # | Run # | Onset (° C.) | (° C.) |
| 1 | 1 | −22.4 | −12.1 |
| 1 | 2 | −22.2 | −12.6 |
| 2 | 1 | −20.2 | −11.8 |

The product underwent partial collapse from collapse onset until total collapse. The annealing did not result in a significant change in drying dynamic or critical temperature. The average collapse onset in method 1 was −22.3° C.

Based on the critical temperatures observed during FDM and DSC analysis, it was determined that the product temperature should be maintained below −25.3° C. during initial primary drying in order to maintain cake structure without collapse or meltback. This temperature incorporates a safe zone of 3 degrees from the determined critical temperature.

### Example 6: Stability Evaluation of Lyophilization Formulations

### I. Stability in Solution

The stability of Formulation 16 in solution was evaluated in this study. The percent related impurity was determined by dividing the area under the curve (AUC) of the related impurity peaks by the AUC of the main Compound 1 peak.

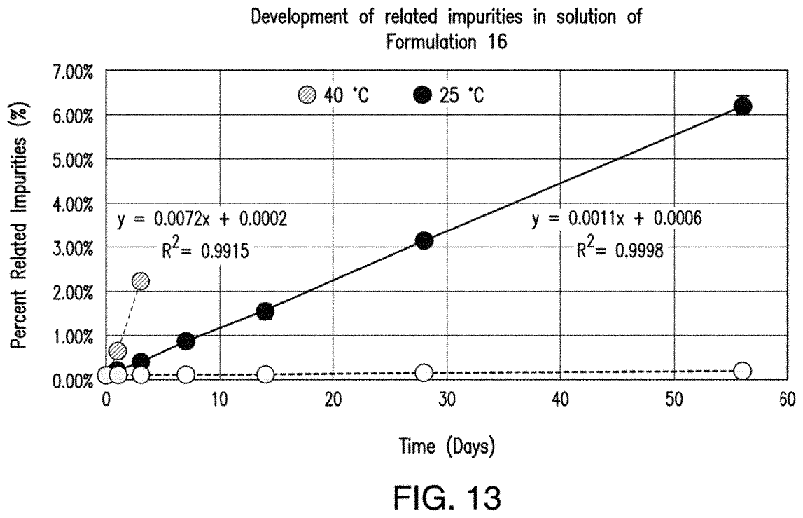

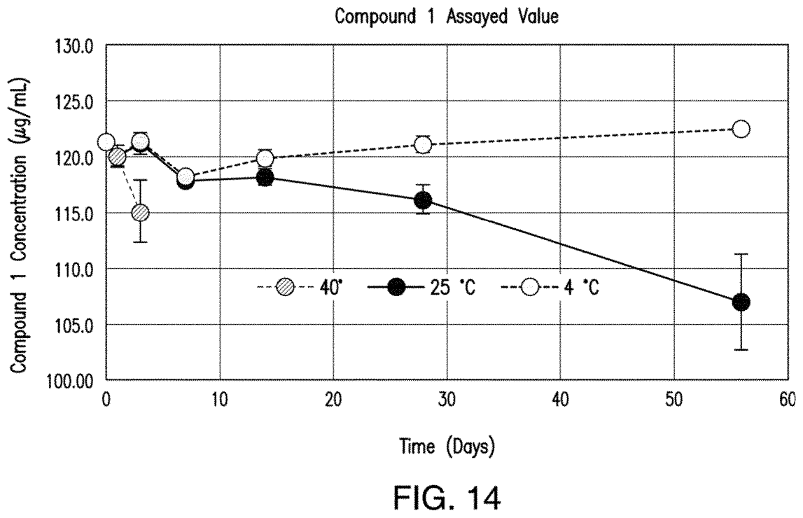

FIG. 13 demonstrates the appearance of related impurities in solutions of Formulation 16 stored at 4-5° C., 25° C./60% RH, and 40° C./75% RH. A strong temperature-dependence on the appearance of related impurities was observed. As shown in FIG. 13, the related impurities developed linearly over time. As shown in FIG. 14, the related impurities were accounted for by the reduction in assayed Compound 1 potency from a mass balance perspective. Evaluation of the 40° C./75% RH sample was terminated after 3 days because the solution became cloudy.

II. Long Term Stability of Lyophilized Formulations

The long-term storage stability of lyophilized formulations at different HSA:Compound 1 ratios, with or without sucrose, and with HSA from two different sources (Novozymes, recombinant human albumin, and Grifols, human albumin from serum) was evaluated in this study. Formulations 7-12 described in Tables 14-15 were evaluated.

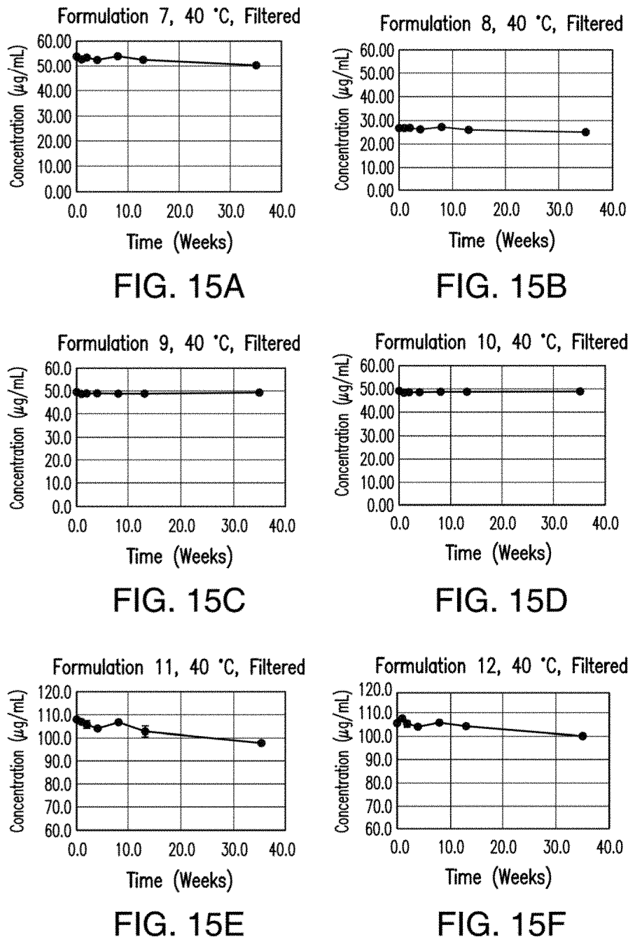

Samples from each formulation were placed on stability at 5° C., 25° C./60% RH, and 40° C./75% RH. Samples were assayed initially (t=0) to establish a baseline. Samples were removed from each storage condition after 1 week, 2 weeks, 1 month, 2 months, 3 months, and 8 months, reconstituted, filtered, and assayed for Compound 1 and human albumin concentration, as well as related impurities. In all lyophilized samples at all storage conditions, no related impurities were observed. Furthermore, there were no differences for the assayed values for all components, between the filtered and untiltered samples. As seen in FIGS. 15A-15F, for samples stored at 40° C./75% RH, formulation without sucrose (Formulation 7 (FIG. 15A), Formulation 8 (FIG. 15B), Formulation 11(FIG. 15E) and Formulation 12 (FIG. 15F)) saw a slight decrease in Compound 1 potency while formulations with sucrose (Formulation 9 (FIG. 15C) and Formulation 10 (FIG. 15D)) saw no change in assayed Compound 1 potency. Similar trends were found for samples stored at 5° C. and at 25° C./60% RH.

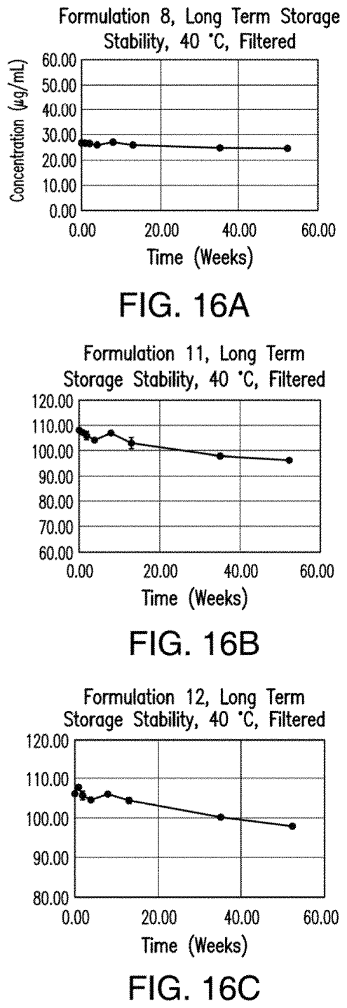

FIGS. 16A, 16B and 16C show long term stability of for samples stored at 40° C./75% RH for Formulations 8, 11 and 12, respectively.

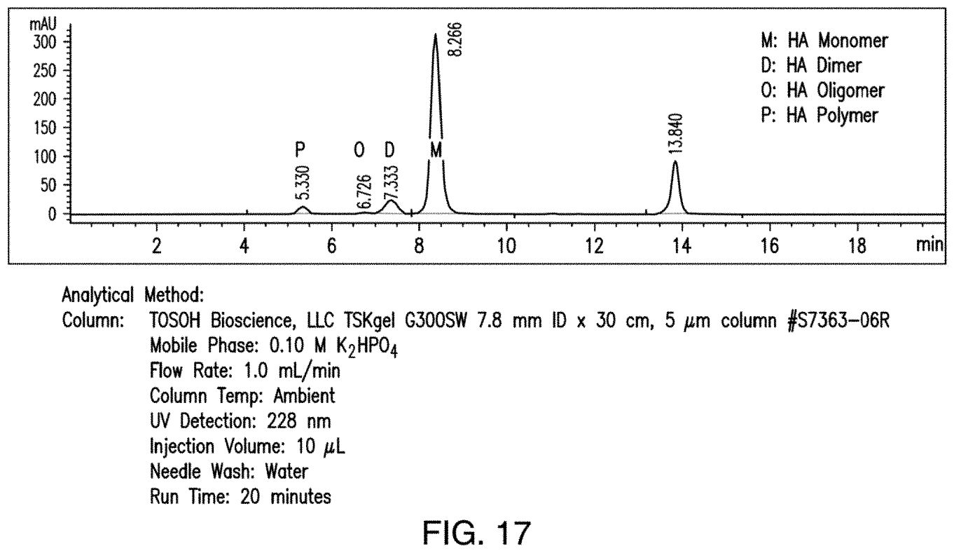

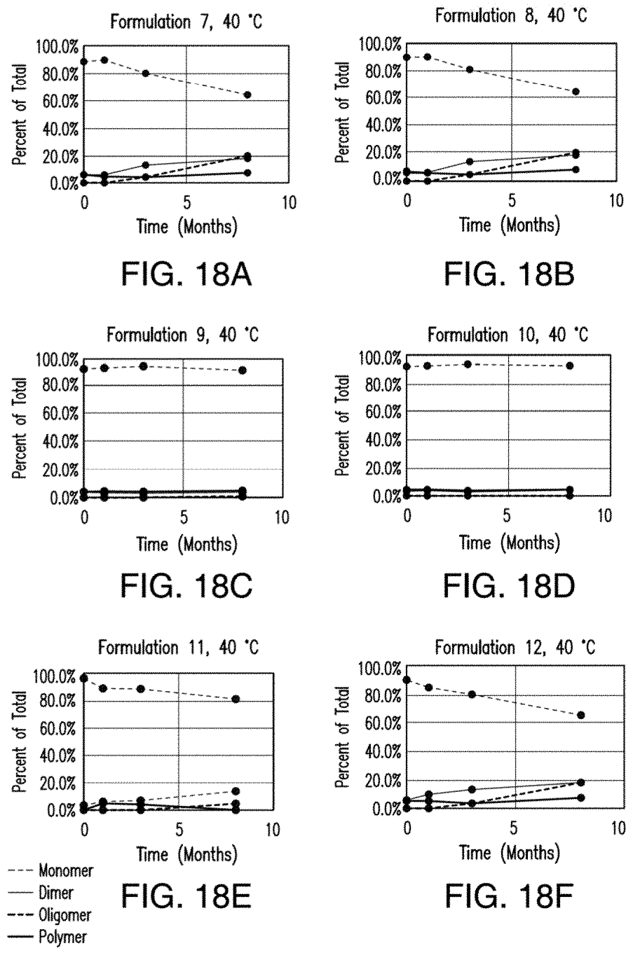

While performing the human albumin assay for total human albumin concentration, the composition of the protein was also quantified in terms of monomer, dimer, oligomer, and polymer fractions. FIG. 17 provides an HPLC chromatogram providing elution times for monomer, dimer, oligomer, and polymer fractions of human album. It was found that, at 40° C./75% RH, for the formulations without sucrose (Formulation 7 (FIG. 18A), Formulation 8 (FIG. 18B), Formulation 11(FIG. 18E) and Formulation 12 (FIG. 18F), the human albumin aggregated over time, indicated by the decrease in the monomer fractions and the corresponding increase in the dimer, oligomer, and polymer fractions. The composition of HA in the formulations with sucrose (Formulation 9 (FIG. 18C) and Formulation 10 (FIG. 18D), stored at 40° C./75% RH, remained largely unchanged for the duration of the study, suggesting that sucrose acts as an important cryoprotectant, not only for maintaining drug potency, but also for preventing human albumin aggregation. The same aggregation that was observed at the 40° C./75% RH condition was not observed for samples stored at 5° C., and only minimally observed at samples stored at 25° C., suggesting that temperature and/or relative humidity are strong drivers for aggregation of human albumin monomers into dimers, oligomers, and polymers.

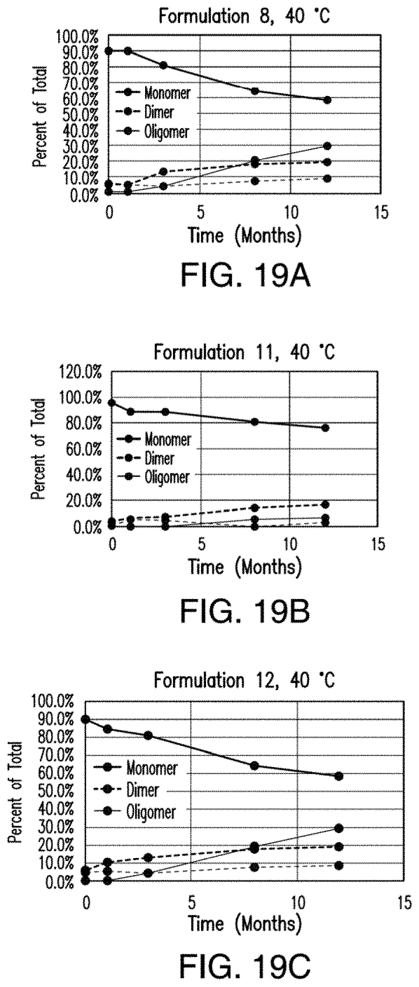

FIGS. 19A, 19B and 19C show the composition of human albumin quantified in terms of monomer, dimer, oligomer, and polymer fractions, for samples stored at 40° C./75% RH for Formulations 8, 11 and 12, respectively.

### Example 7: Assessment of Compound 1 Solubility in Formic Acid/Acetic Acid Mixtures

In this study, Compound 1 solubility in formic acid/acetic acid (FA/AcOH) mixtures at temperatures up to 60° C. was determined. FA/AcOH mixtures ranging from 100% FA to 70%/30% FA/AcOH in decrements of 10% were used.

Solutions of FA/AcOH at ratios described in Table 39 were used. 500 μL of each solution was added individually to vials containing >150 mg of Compound 1, and placed into each temperature condition (12 samples). Samples were taken at each time point (4, 24, and 48 h) and assayed (36 total). Samples were spun down briefly (500 g, 4 min) to precipitate all solids and 25 μL of solution were taken for assay.

TABLE 39

| | | 90%/10% | 80%/20% | 70%/30% |
|---|---|---|---|---|
| | 100% FA | FA/AcOH | FA/AcOH | FA/AcOH |
| 4° C. | 4, 24, 48 h | 4, 24, 48 h | 4, 24, 48 h | 4, 24, 48 h |
| 25° C. | 4, 24, 48 h | 4, 24, 48 h | 4, 24, 48 h | 4, 24, 48 h |
| 40° C. | 4, 24, 48 h | 4, 24, 48 h | 4, 24, 48 h | 4, 24, 48 h |

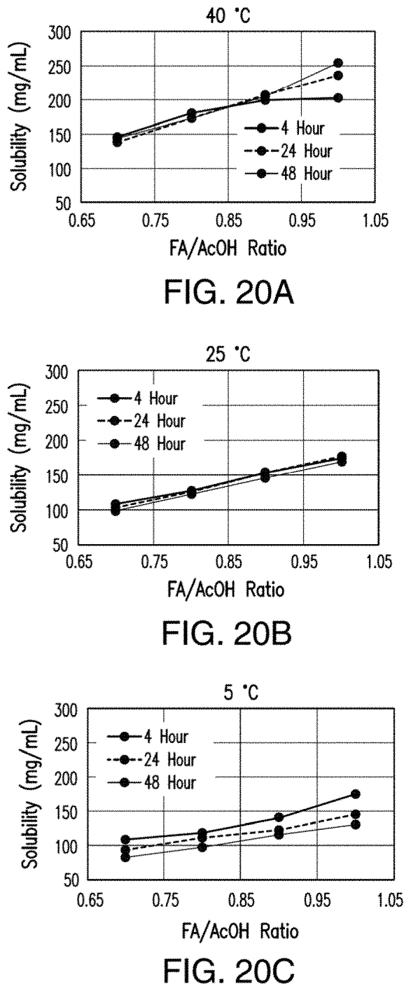

FIGS. 20A, 20B and 20C provide plots for solubility of Compound 1 at various FA/AcOH mixtures studied. As seen from FIGS. 20A-20C, solubility of Compound 1 is maximized in 100% FA. As seen from FIG. 20A, heating to 40° C. for >24 hours results in Compound 1 in 100% FA approaching its true solubility of >250 mg/mL. It was observed that heating to 60° C. allowed to reach the same solubility of >250 mg/mL within 1 hour.

### Example 8: Reconstitution Time Study

The effect of pH, concentration, fill volume and drug content on reconstitution time was studied for Formulations A, B, C and D described in Table 40.

TABLE 40

| Parameter | A | B | C | D |
|---|---|---|---|---|
| Compound 1 | 100 mg/mL | 100 mg/mL | 200 mg/mL | 0 mg/mL |
| concentration | | | | |
| HSA | 50 mg/mL | 50 mg/mL | 100 mg/mL | 100 mg/mL |
| concentration | | | | |
| Sucrose | 66 mg/mL | 66 mg/mL | 132 mg/mL | 132 mg/mL |
| concentration | | | | |
| Formic acid/ | 4.575 mg/mg | 4.575 mg/mg | 4.575 mg/mg | 4.575 mg/mg |
| Compound 1 | | | | |
| Acetic acid/ | 3.453 mg/mg | 3.453 mg/mg | 3.453 mg/mg | 3.453 mg/mg |
| Compound 1 | | | | |
| Citrate buffer | 20 mM | 20 mM | 40 mM | 40 mM |
| concentration | | | | |
| pH | 3.8 | 5.0 | 5.0 | 3.8 |
| Fill volume | 25 mL | 25 mL | 12.5 mL | 12.5 mL |
| Vial size | 50 cc | 50 cc | 50 cc | 50 cc |
| Reconstitution | WFI | WFI | WFI | WFI |
| vehicle | | | | |
| Volume of | 23.2 | 23.2 | 23.2 | 23.2 |
| vehicle added | | | | |
| Recon | 100 mg/mL | 100 mg/mL | 100 mg/mL | 0 mg/mL |
| Compound 1 | | | | |
| concentration | | | | |
| Recon HSA | 5% | 5% | 5% | 5% |
| concentration | | | | |

The formulations were were prepared according to the flow chart provided in FIG. 20.

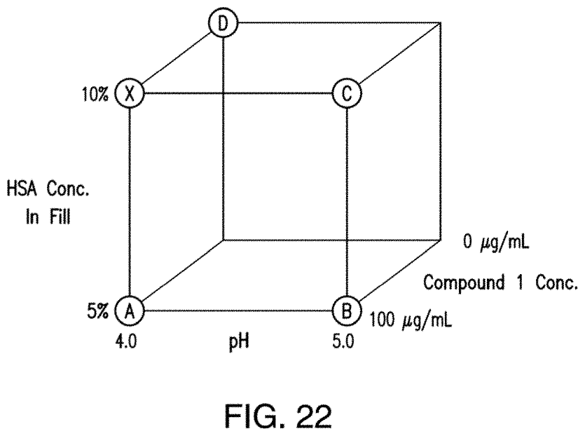

The effect of pH (4 or 5), fill concentration (1× or 2×) and drug content (0 or 100 μg/mL) on reconstitution time was determined using the following procedure (as shown in FIG. 22):
Test reconstitution time of 5 vials from day 1 with 23.2 mL WFI
Label day 1 vials as formulation X
Reconstitute vials from day 1 batch with 10 mL
Blend and adjust pH to create formulations A, B and C
Prepare formulation D with all excipients except Compound 1
Fill into 50 cc vials and lyophilize with the following cycle
Reconstitute all with 23.2 mL WFI and occasional swirling

The formulations were lyophilized as follows:
Initiate lyophilization on day 11
Equilibrate to 5° C. for 1 hr
Ramp to −45° C. at 0.25° C./min
Freeze at −45° C. for 4 hrs
Ramp to −20° C. at 0.25° C./min
1° drying at −20° C. for 60 hrs at 100 mTorr
Ramp to +25° C. at 0.1° C./min
2° drying at +25° C. for 12 hrs at 200 mTorr
Remove from lyophilizer on day 15.

The effect of pH, fill volume and drug content on reconstitution time is provided in Table 41.

TABLE 41

| | | | | | Reconstitution | | |
|---|---|---|---|---|---|---|---|
| Formulation | | | | | Time, minutes | | |
| ID | n | pH | Fill | Drug | Average | Std. Dev. | pH |
| X | 5 | 3.8 | 2X | Yes | 27.7 | 2.9 | 3.80 |
| A | 3 | 3.8 | 1X | Yes | 19.2 | 3.9 | 3.84 |
| B | 4 | 5.0 | 1X | Yes | 10.5 | 1.5 | 4.98 |
| C | 4 | 5.0 | 2X | Yes | 17.6 | 5.0 | 4.97 |
| D | 3 | 3.8 | 2X | No | 35 | 2.0 | 3.70 |

As seen from data in Table 41, Formulation B reconstituted most rapidly, where the slowest vial reconstituted in 12 minutes. Foam cleared within 30 minutes for formulation B. Presence of drug concentration did not slow reconstitution. Formulations at pH 5 reconstituted faster than those at pH 3.8. Formulations with 1×concentration fills reconstituted faster than with 2× fills.

### Example 9: Monkey PK Study

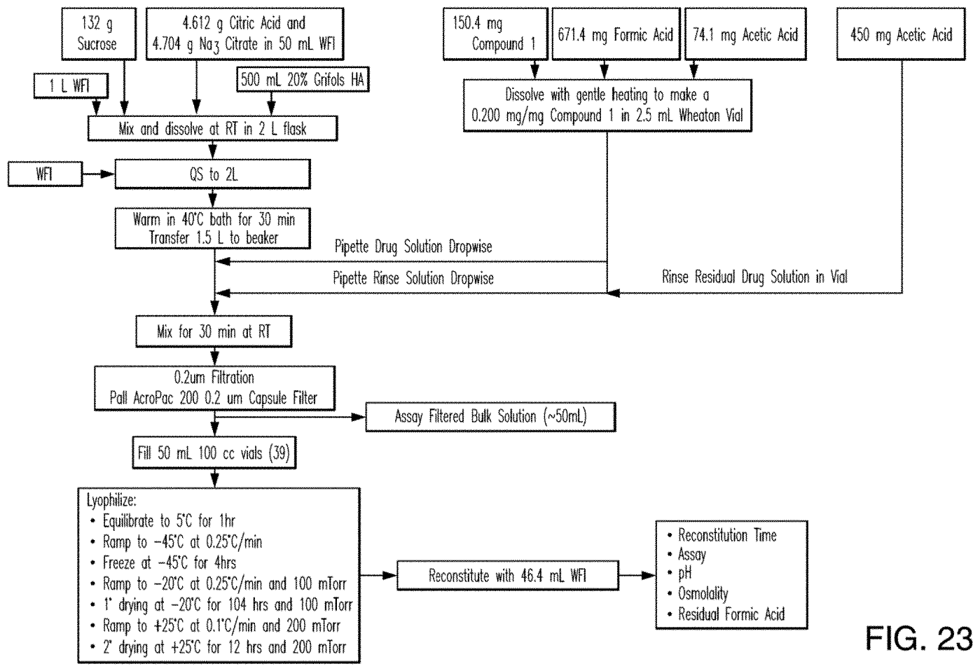

A formulation having the composition described in Table 42 was prepared for monkey PK studies. A flow diagram for preparation and lyophilization of the formulation is provided in FIG. 23.

TABLE 42

| Drug Product Parameter | Target Value |
|---|---|
| Compound 1 concentration | 100 mg/mL |
| Formic acid/Compound 1 | 4.5 mg/mg |
| Acetic acid/Compound 1 | 3.5 mg/mg |
| HSA concentration | 50 mg/mL |
| Sucrose concentration | 66 mg/mL |
| Citrate buffer concentration | 20 mM |
| pH of HSA/sucrose/citrate solution | 5.0 |
| Fill volume | 50 mL |
| Vial size | 100 cc |
| Reconstitution vehicle | WFI |
| Volume of vehicle added | 46.4 |
| Dose/vial | 5 mg |
| HSA/vial | 2.5 g |

Monkey PK study was conducted as follows: 4 monkeys (2 males/2 females) were used in a crossover study design to evaluate multiple formulations. Single dose of 0.3 mg/kg (3 mL/kg; 30 minute IV infusion) was administered. Monkeys were not fasted prior to dosing. PK samples were collected at 0, 0.083, 0.25, 0.5, 1, 2, 4, 6, 8, 12 and 24 hours post-infusion.

Formulation Ib provided in Table 43 (described in WO 2019/006299) and HSA formulation described in Table 42 were evaluated in this study.

TABLE 43

| | Formulation Ib |
|---|---|
| Compound 1 | 1.0 mg/vial |
| Citric acid anhydrous, USP | — |
| Sodium citrate anhydrous, USP | — |
| Kleptose ® HPB (HP-β-CD), | 800 mg/vial |
| parenteral grade | |
| Dimethyl sulfoxide (processing aid) | — |
| Formic acid (processing aid) | Partially removed upon drying |
| Water for injection (processing aid) | Removed upon drying |

Monkey PK data for both formulations are provided in Table 44 and FIG. 24.

TABLE 44

| | | C_{5 min} | | |
|---|---|---|---|---|
| Formulation | Dose (mg/kg) | (ng/mL) | AUCₜ (ng*hr/mL) | t_{1/2} (hr) |
| Ib | 0.3 | 230 (17) | 365 (24) | 2.4 (9.3) |
| HSA | 0.3 | 270 (27) | 534 (27) | 2.1 (4.5) |

Data are mean of 4 animals (and % CV)

The data in Table 44 and FIG. 23 demonstrate that the HSA formulation resulted in higher exposures than Formulation lb. Half-lives for both formulations were about the same. Inter-animal variability was similar across both formulations.

### Example 10: Formulations with Sucrose and Trehalose and Human Albumin Formulations

Formulations with sucrose or trehalose (2% or 1%) and mannitol (Formulations 17-24) were prepared with human albumin. Table 45 below provides compositions for each of the formulation in bulk solution.

For each of Formulations 17-24, mass of each component in the vial is provided in Table 46 below. Formulations 17-21 are sucrose formulations and Formulations 22-24 are trehalose formulations.

For each of Formulations 17-24, mass fraction of each component in the lyophilized product s provided in Table 47 below.

TABLE 45

| Formulation No. | 17 | 18 | 19 | 20 | 21 | 22 | 23 | 24 |
|---|---|---|---|---|---|---|---|---|
| Compound 1 Concentration (μg/mL) | 200 | 100 | 100 | 100 | 100 | 100 | 100 | 100 |
| Human Albumin Concentration (mg/mL) | 100 | 50 | 50 | 50 | 50 | 50 | 50 | 50 |
| Albumin/Compound 1 Ratio | 500 | 500 | 500 | 500 | 500 | 500 | 500 | 500 |
| Sucrose/Trehalose Concentration (mg/mL) | 132 | 66 | 66 | 20 | 10 | 20 | 10 | 20 |
| Citric Acid Concentration (mM) | 40 | 18.75 | 18.75 | 18.75 | 18.75 | 18.75 | 20 | 20 |
| Mannitol Concentration (mg/mL) | | | | 20 | 25 | 20 | 25 | 20 |
| pH prior to formic acid addition | 4.0 | 5.0 | 5.0 | 5.0 | 5.0 | 5.0 | 5.0 | 5.0 |
| Formic acid Concentration (μg/mL) | 0.92 | 0.45 | 0.45 | 0.45 | 0.45 | 0.45 | 0.45 | 0.45 |
| Acetic acid Concentration (μg/mL) | 0.67 | 0.66 | 0.35 | 0.20 | 0.20 | 0.20 | 0.20 | 0.20 |
| Sodium N-acetyltryptophanate Conc. (mM) | 8.0 | 4.0 | 4.0 | 4.0 | 4.0 | 4.0 | 4.0 | 4.0 |
| Sodium caprylate Conc. (mM) | 8.0 | 4.0 | 4.0 | 4.0 | 4.0 | 4.0 | 4.0 | 4.0 |
| pH of fully formulated solution | 3.89 | | | 4.63 | 4.65 | 4.65 | 4.64 | |
| Tonicity of fully formulated solution | 796 | | | 306 | 304 | 302 | 299 | |
| (mOsm/kg) | | | | | | | | |
| Vial size (cc) | 50 | 100 | 100 | 10 | 10 | 10 | 10 | 100 |
| Fill Volume (mL) | 12.5 | 50 | 50 | 5 | 5 | 5 | 5 | 60 |
| Reconstituted Volume (mL) | 25 | 50 | 50 | 5 | 5 | 5 | 5 | 60 |
| Volume of media to reconstitute (mL) | 23.2 | 46.4 | 45.6 | 4.51 | 4.53 | 4.51 | 4.53 | 56.0 |
| Reconstitution media | WFI | WFI | WFI | WFI | WFI | WFI | WFI | WFI |
| pH of reconstituted solution | 3.86 | 4.76 | 4.72 | | | | | |
| Tonicity of reconstituted solution | 318 | 336 | 350 | | | | | |
| (mOsm/kg) | | | | | | | | |

TABLE 46

| Formulation No. | 17 | 18 | 19 | 20 | 21 | 22 | 23 | 24 |
|---|---|---|---|---|---|---|---|---|
| Compound 1 per vial (mg) | 2.5 | 5 | 5 | 0.5 | 0.5 | 0.5 | 0.5 | 6 |
| Human Albumin per vial (mg) | 1250 | 2500 | 2500 | 250 | 250 | 250 | 250 | 3000 |
| Sucrose/Trehalose per vial (mg) | 1650 | 3300 | 3300 | 100 | 50 | 100 | 50 | 1200 |
| Mannitol per vial | — | — | — | 100 | 125 | 100 | 125 | 1200 |
| Citric acid per vial (mg) | 96.1 | 192.1 | 192.1 | 19.2 | 19.2 | 19.2 | 19.2 | 230.5 |
| Sodium chloride per vial (mg) | 53.0 | 105.9 | 105.9 | 10.6 | 10.6 | 10.6 | 10.6 | 127.1 |
| Sodium N-acetyltryptophanate | 26.8 | 53.6 | 53.6 | 5.4 | 5.4 | 5.4 | 5.4 | 64.4 |
| per vial (mg) | | | | | | | | |
| Sodium caprylate per vial (mg) | 16.6 | 33.2 | 33.2 | 3.3 | 3.3 | 3.3 | 3.3 | 39.9 |
| Formic acid per vial (mg) | 11.44 | 22.50 | 22.50 | 2.25 | 2.25 | 2.25 | 2.25 | 27.00 |
| Acetic acid per vial (mg) | 8.43 | 33.10 | 17.50 | 1.00 | 1.00 | 1.00 | 1.00 | 12.00 |
| Total mass per vial (mg) | 3114.8 | 6245.5 | 6229.9 | 492.2 | 467.2 | 492.2 | 467.2 | 5906.9 |

TABLE 47

| Formulation No. | 17 | 18 | 19 | 20 | 21 | 22 | 23 | 24 |
|---|---|---|---|---|---|---|---|---|
| Compound 1 per vial (% w/w) | 0.080% | 0.080% | 0.080% | 0.10% | 0.11% | 0.10% | 0.11% | 0.10% |
| Human Albumin per vial (% w/w) | 40.131% | 40.029% | 40.129% | 50.79% | 53.51% | 50.79% | 53.51% | 50.79% |
| Sucrose/Trehalose per vial (% w/w) | 52.972% | 52.838% | 52.970% | 20.32% | 10.70% | 20.32% | 10.70% | 20.32% |
| Mannitol per vial (% w/w) | — | — | — | 20.32% | 26.75% | 20.32% | 26.75% | 20.32% |
| Citric acid per vial (% w/w) | 3.084% | 3.076% | 3.084% | 3.90% | 4.11% | 3.90% | 4.11% | 3.90% |
| Sodium chloride per vial (% w/w) | 1.700% | 1.696% | 1.700% | 2.15% | 2.27% | 2.15% | 2.27% | 2.15% |
| Sodium N-acetyltryptophanate | 0.861% | 0.859% | 0.861% | 1.09% | 1.15% | 1.09% | 1.15% | 1.09% |
| per vial (% w/w) | | | | | | | | |
| Sodium caprylate per vial (% w/w) | 0.534% | 0.532% | 0.534% | 0.68% | 0.71% | 0.68% | 0.71% | 0.68% |
| Formic acid per vial % w/w | 0.367% | 0.360% | 0.361% | 0.46% | 0.48% | 0.46% | 0.48% | 0.46% |
| Acetic acid per vial (% w/w) | 0.270% | 0.530% | 0.281% | 0.20% | 0.21% | 0.20% | 0.21% | 0.20% |
| Total mass per vial (% w/w) | 0.080% | 0.080% | 0.080% | 0.10% | 53.51% | 0.10% | 0.11% | 0.10% |

Formulations 17-24 were lyophilized using cycles provided in Tables 48-51 below:

TABLE 48
Lyophilization cycle for Formulation 17

| | Temperature | Duration | Pressure |
|---|---|---|---|
| | [° C.] | [minutes] | [mTorr] |
| Pre-cooling hold | 5 | 60 | Atmos. |
| Freezing ramp | −45 | 200 | Atmos. |
| Freezing hold for annealing | | | |
| Annealing Ramp | | | |
| Annealing Hold | | | |
| Freezing ramp - 2 | | | |
| Freezing hold | −45 | 240 | Atmos. |
| Vacuum equilibration | −45 | 10 | 100 |
| Primary drying ramp | −20 | 100 | 100 |
| Primary drying hold | −20 | | 100 |
| Secondard drying ramp | 25 | 450 | 200 |
| Secondary drying hold | 25 | 720 | 200 |
| High temperature drying ramp | 60 | 400 | 200 |
| High temperature drying hold | 60 | 720 | 200 |
| Ramp to 25° C. | 25 | Uncontrolled | 350 |

TABLE 49
Lyophilization cycle for Formulation 18

| | Temperature | Duration | Pressure |
|---|---|---|---|
| | [° C.] | [minutes] | [mTorr] |
| Pre-cooling hold | 5 | 60 | Atmos. |
| Freezing ramp | −45 | 200 | Atmos. |
| Freezing hold for annealing | | | |
| Annealing Ramp | | | |
| Annealing Hold | | | |
| Freezing ramp - 2 | | | |
| Freezing hold | −45 | 240 | Atmos. |
| Vacuum equilibration | −45 | 10 | 100 |
| Primary drying ramp | −20 | 100 | 100 |
| Primary drying hold | −20 | | 100 |
| Secondard drying ramp | 25 | 450 | 200 |
| Secondary drying hold | 25 | 720 | 200 |
| High temperature drying ramp | 60 | 400 | 200 |
| High temperature drying hold | 60 | 720 | 200 |
| Ramp to 25° C. | 25 | Uncontrolled | 350 |

TABLE 50
Lyophilization cycle for Formulation 19

| | Temperature | Duration | Pressure |
|---|---|---|---|
| | [° C.] | [minutes] | [mTorr] |
| Pre-cooling hold | 5 | 60 | Atmos. |
| Freezing ramp | −45 | 200 | Atmos. |
| Freezing hold for annealing | | | |
| Annealing Ramp | | | |
| Annealing Hold | | | |
| Freezing ramp - 2 | | | |
| Freezing hold | −45 | 240 | Atmos. |
| Vacuum equilibration | −45 | 10 | 100 |
| Primary drying ramp | −20 | 100 | 100 |
| Primary drying hold | −20 | | 100 |
| Secondard drying ramp | 25 | 450 | 200 |
| Secondary drying hold | 25 | 720 | 200 |
| High temperature drying ramp | 60 | 400 | 200 |
| High temperature drying hold | 60 | 720 | 200 |
| Ramp to 25° C. | 25 | Uncontrolled | 350 |

TABLE 51
Lyophilization cycle for Formulations 20-24

| | Temperature | Duration | Pressure |
|---|---|---|---|
| | [° C.] | [minutes] | [mTorr] |
| Pre-cooling hold | 5 | 30 | Atmos. |
| Freezing ramp | −45 | 200 | Atmos. |
| Freezing hold for annealing | −45 | 180 | Atmos. |
| Annealing Ramp | −15 | 60 | Atmos. |
| Annealing Hold | −15 | 180 | Atmos. |
| Freezing ramp - 2 | −45 | 60 | Atmos. |
| Freezing hold | −45 | 150 | Atmos. |
| Vacuum equilibration | | | |
| Primary drying ramp | −15 | 60 | Atmos. |
| Primary drying hold | −15 | | 100 |
| Secondard drying ramp | 25 | 400 | 200 |
| Secondary drying hold | 25 | 720 | 200 |
| High temperature drying ramp | | | |
| High temperature drying hold | | | |
| Ramp to 25° C. | 25 | Uncontrolled | 350 |

Formulations 20-23 were evaluated, with an addition of an annealing step during lyophilization. All 4 formulations achieved <5 min reconstitution time.

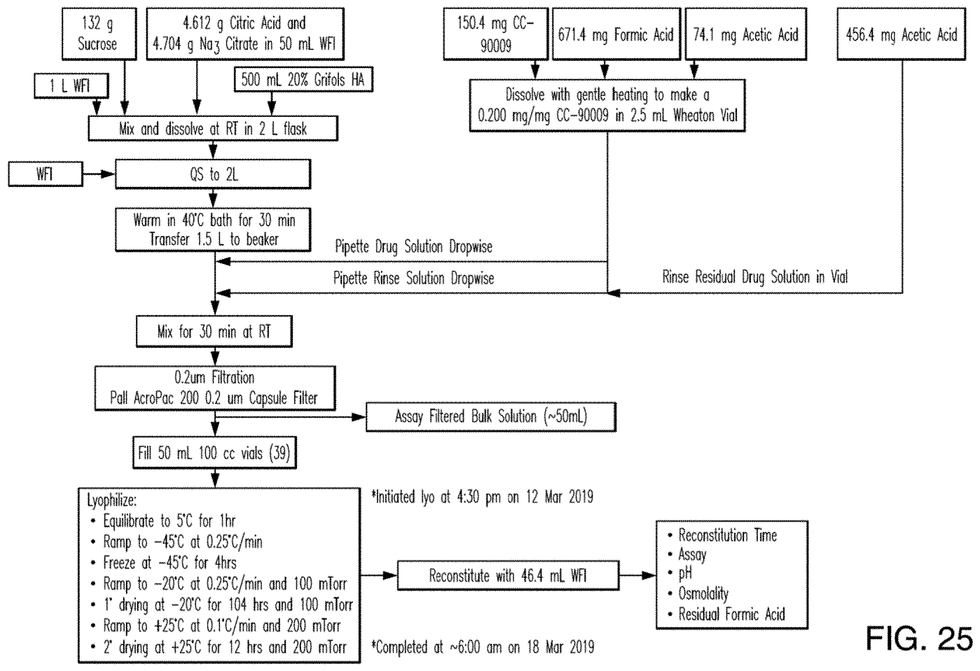

FIG. 25 provides a flow diagram for preparation of a of a large scale batch of Formulation 24.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed:

1. A formulation comprising: 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof in an amount of about 1 to 1.3%, citrate buffer in an amount of about 9 to 12% and mannitol in an amount of about 85 to 90%, based on the total weight of the formulation.

2. The formulation of claim 1 comprising: 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof in an amount of about 1.0%, based on the total weight of the formulation.

3. The formulation of claim 1, comprising citrate buffer in an amount of about 10.63% based on the total weight of the formulation.

4. The formulation of claim 1, wherein the citrate buffer comprises citric acid monohydrate and sodium citrate dihydrate.

5. The formulation of claim 1, comprising mannitol in an amount of about 88% based on the total weight of the formulation.

6. The formulation of claim 1, comprising 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof in the amount of about 1.0% citrate buffer in an amount of about 10.63% and mannitol in an amount of about 88%, based on the total weight of the formulation.

7. The formulation of claim 1 comprising 1 mg (2-(4-chlorophenyl)-N-((2-(2,6-dioxopiperidin-3-yl)-1-oxoisoindolin-5-yl)methyl)-2,2-difluoroacetamide), 5.24 mg citric acid monohydrate, 4.4 mg sodium citrate dihydrate and 80 mg mannitol.

8. A formulation comprising: 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof, a citrate buffer, human albumin, and sucrose.

9. The formulation of claim 8, comprising about 0.03% to about 0.25% 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof, about 30.00% to 90.00% human albumin, about 20.00% to 60.00% sucrose, and about 1.00% to 8.00% citric acid.

10. The formulation of claim 8 further comprising about 1% to 9% sodium chloride.

11. The formulation of claim 8 further comprising about 0.5% to 2.5% sodium N acetyltryptophanate.

12. The formulation of claim 8 further comprising about 0.3% to 1.2% sodium caprylate.

13. The formulation of claim 8 comprising about 0.03% to 0.05% 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof, about 38.00% to 47.00% human albumin, about 45.00% to 55.00% sucrose, and about 30.00% to 4.00% citric acid.

14. The formulation of claim 8 comprising about 0.042% 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof, about 42.29% human albumin, about 50.75% sucrose, about 30.65% citric acid, about 1.79% sodium chloride, about 0.91% sodium N acetyltryptophanate and about 0.56% sodium caprylate.

15. A formulation comprising: 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof, a citrate buffer, human albumin, trehalose and mannitol.

16. The formulation of claim 15 comprising about 0.08% to 0.12% 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof, about 40.00% to 55.00% human albumin, about 10.00% to 25.00% trehalose, about 15% to 30% mannitol, about 3.00% to 4.50% citric acid, about 1.50% to 2.50% sodium chloride, about 0.80% to 1.50% sodium N-acetyltryptophanate, about 0.50% to 1.00% sodium caprylate, about 0.30% to 0.50% formic acid and about 0.20% to 0.60% acetic acid based on the total weight of the formulation.

17. The formulation of claim 16 comprising about 0.1% 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof, about 50.79% human albumin, about 20.32% trehalose, about 20.32% mannitol, about 30.90% citric acid, about 2.15% sodium chloride, about 1.09% sodium N-acetyltryptophanate, about 0.68% sodium caprylate, about 0.46% formic acid and about 0.20% acetic acid based on the total weight of the formulation.

18. The formulation of claim 1 comprising (2-(4-chlorophenyl)-N-((2-(2,6-dioxopiperidin-3-yl)-1-oxoisoindolin-5-yl)methyl)-2,2-difluoroacetamide).

19. The formulation of claim 1, wherein the formulation is an aqueous formulation further comprising a diluent.

20. The formulation of claim 19, wherein the diluent comprises PEG400, ethanol, and water for injection.

21. The formulation of claim 20, wherein the diluent comprises PEG400, ethanol, and water for injection in a volume ratio of 50:10:40.

22. The formulation of claim 19, comprising 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof in an amount of about 0.1 mg/mL.

23. The formulation of claim 19, comprising 2-(4-chlorophenyl)-N-((2-(2,6-dioxopiperidin-3-yl)-1-oxoisoindolin-5-yl)methyl)-2,2-difluoroacetamide, or a stereoisomer or mixture of stereoisomers, pharmaceutically acceptable salt, tautomer, prodrug, isotopologue, solvate, hydrate, co-crystal, clathrate, or polymorph thereof in an amount of about 0.1 mg/mL, mannitol in an amount of 8.0 mg/mL, citric acid monohydrate in an amount of about 0.52 mg/mL, and sodium citrate dihydrate in an amount of about 0.44 mg/mL.

24. The formulation of claim 19, wherein the formulation has a pH in a range from about 4 to 5.

25. The formulation of claim 8, wherein the formulation is an aqueous formulation further comprising a diluent.

26. The formulation of claim 25, wherein the diluent comprises water.

27. The formulation of claim 15, wherein the formulation is an aqueous formulation further comprising a diluent.

28. The formulation of claim 27, wherein the diluent comprises water.

29. A vial comprising the formulation of claim 1.

30. A method of treating a cancer in a mammal, wherein the method comprises administering the formulation of claim 1 to the mammal.

31. A method of treating a cancer in a mammal, wherein the method comprises administering the aqueous formulation of claim 19 intravenously.

32. The method of claim 30, wherein the cancer is leukemia.

33. The method of claim 32, wherein the leukemia is chronic lymphocytic leukemia, chronic myelocytic leukemia, acute lymphoblastic leukemia or acute myeloid leukemia.

34. The method of claim 32, wherein the leukemia is an acute myeloid leukemia.

35. The method of claim 32, wherein the leukemia is relapsed, refractory or resistant.

36. The method of claim 32, further comprising administering a therapeutically effective amount of another second active agent or a supportive care therapy.

37. The method of claim 36, wherein the other second active agent is a therapeutic antibody that specifically binds to a cancer antigen, a hematopoietic growth factor, a cytokine, anti-cancer agent, an antibiotic, a cox-2 inhibitor, an immunomodulatory agent, an immunosuppressive agent, a corticosteroid or a pharmacologically active mutant or derivative thereof.

38. The method of claim 37, wherein the second active agent is selected from a glucocorticoid receptor agonist, an IL-1β receptor antagonist, an interleukin-1β blocker, a JAK inhibitor, a FLT3 inhibitor, an mTOR inhibitor, a spiceosome inhibitor, an ERK inhibitor, an LSD1 inhibitor, an SMG1 inhibitor, a BH3 mimetic, and a topoisomerase inhibitor.

39. A method of treating a myeloproliferative neoplasm in a mammal, wherein the method comprises administering the formulation of claim 1.

40. The method of claim 39 further comprising administering a JAK inhibitor.

41. A method of treating a cancer selected from breast cancer, neuroendocrine tumor, and renal cell carcinoma in a mammal, wherein the method comprises administering the formulation of claim 1.

42. The method of claim 41 further comprising administering a second agent selected from everolimus, temsirolimus, 1-ethyl-7-(2-methyl-6-(1H-1,2,4-triazol-3-yl)pyridin-3-yl)-3,4-dihydropyrazino[2,3-b]pyrazin-2(1H)-one and 7-(6-(2-hydroxypropan-2-yl)pyridin-3-yl)-1-((trans)-4-methoxycyclohexyl)-3,4-dihydropyrazino[2,3-b]pyrazin-2(1H)-one to the mammal.

43. A method of treating a leukemia in a mammal, wherein the method comprises administering the formulation of claim 1 in combination with an IDH2 inhibitor to the mammal, wherein the leukemia is characterized by the presence of a mutant allele of IDH2.

44. The method of claim 43, wherein the IDH2 inhibitor is enasidenib or 6-(6-(trifluoromethyl)pyridin-2-yl)-N²-(2-(trifluoromethyl)pyridin-4-yl)-1,3,5-triazine-2,4-diamine.

45. The method of claim 43, wherein the leukemia is an acute myeloid leukemia characterized by the presence of a mutant allele of IDH2.

46. The method of claim 43, wherein the leukemia is relapsed, refractory or resistant.

47. A process for preparing the formulation of claim 1 comprising: dissolving mannitol in tert-butyl alcohol and citrate buffer to obtain a buffer solution, and dissolving (2-(4-chlorophenyl)-N-((2-(2,6-dioxopiperidin-3-yl)-1-oxoisoindolin-5-yl)methyl)-2,2-difluoroacetamide) in the buffer solution to a drug solution.

48. The process of claim 47 further comprising lyophilizing the drug solution to obtain a lyophilized formulation.

49. A process for preparing the formulation of claim 8 comprising: (i) adding a mixture of sucrose and 20% human albumin to a citrate buffer in water to obtain a sucrose/human albumin solution, and (ii) adding a solution of Compound 1 in formic acid to the sucrose/human albumin solution to obtain a drug solution.

50. The process of claim 49 further comprising: filtering the drug solution to obtain a filtered solution, and lyophilizing the filtered solution to obtain a lyophilized formulation.

51. A process for preparing the formulation of claim 15 comprising: (i) adding a mixture of trehalose, mannitol and 20% human albumin to a citrate buffer in water to obtain a trehalose/mannitol/human albumin solution, and (ii) adding a solution of Compound 1 in formic acid to the trehalose/mannitol/human albumin solution to obtain a mixture, and (iii) adding acetic acid to the mixture to obtain a drug solution.

52. The process of claim 51 further comprising: filtering the drug solution to obtain a filtered solution, and lyophilizing the filtered solution to obtain a lyophilized formulation.
